SEQUENCE LISTING (34 sequences)

SEQ ID NO: 1 - DNA, 2019 bp, Homo sapiens
gcggcgcagcaccgcgggctccccgcccgccgctgccgggagtgggacggggccggccgtgagctgcgcaccggctgcgggagccggccggctgctgcagcccatccgcgccacggtgccctaccagctcctgcggggcagccagcacagccccacgcgccccgccgccgccgccgcgctcggcagtctcccagggcccagcggggcccgtggccctagcccgtccagcccgactccaccgccggccactgccccagccgagcaggcgcctcgcgccaagggccgcccgagacggtcccccgagagccagcggaggagcagctcacctgagagacggagtcccggctcgcccgtgtgcagagtggacagaccaaaatctcagcaaattcgaaactctagtacaataaggcgaacctcttctttggatacgataacaggaccttacctcacaggacagtggccacgtgaccctcatgttcactacccttcgtgcatgaaagataaagcgactcagacacctagctgttgggcagaggagggagcagaaaaacgatcacatcagcgctctgcgtcatggggaagtgctgatcaactgaaagagcagattgccaaactcaggcagcagttacagcgcagcaagcagagcagtcggcacagtagagagaaagatcgacagtcacctctccatggcaaccacataccgatcagtcatactcaggctattgggtccaggtcagtccctatgcctctgtcaaacatatccgtgccaaaatcctctgtttcccgtgtgccctgcaatgtagaagggataagtcctgaactggaaaaggtattcatcaaagaaaacaatgggaaggaagaagtatccaagccgttggatataccagatggtcgaagagccccgctccctgcgcactacaggagcagtagtacccgaagcatagatacccagacaccttctgtccaagagcgcagcagtagctgcagcagccactccccttgtgtgtccccattttgtcctccggaatcccaggatggaagtccttgttcaacagaagatttgctgtatgatcgtgataaagacagtgggagtagctcaccgttacccaagtatgcttcatctcccaaaccaaacaacagctacatgttcaaacgggagcccccagagggatgtgagcgagtgaaggtctttgaggaaatggcgtctcgtcagcctatctcggcccctctcttttcatgtcctgacaaaaacaaggttaatttcatcccaaccggatcagctttctgtcctgtaaaacttctaggccctctcttacctgcctctgacctgatgctcaagaactctcctaattctggccagagctcagctctggccacactaaccgtagagcagctctcctcccgggtctccttcacgtccctttctgatgacaccagcaccgcagactccctggagccctctgtccagcagccatctcagcagcagcagctcctgcaggatttgcaggcagaggaacacatctccactcagaactatgtgatgatctaaagcagagggggagctggcctccgcccatgttccatggatcgggaatgagatctcagacatctatctgcatggagtgacaaactttctgaacaccaccaccaacagcaaaatacttagcatcataaaatagctattaacactgatcttggcagggaccgacttctattcagcagtttttgtggaaagcagtaatgcttgcaaaaatgtgtgtgtcattcagcatttaagtggagactatgcatttcatagtatgtctgacagactagtactgtgtcctgtgttttgttccaaatttttcagtatgaataagctctacttcaaaaagttgcctgtctaagtagaaaatgtcttgctgtgttttgtcctatggaaaatactgtacttcaggattatgtttacaattgatccaggtgtttgtttctaacttctataatacatacaatgcaaaaaaaaaaaaaaagggcggccgc

SEQ ID NO: 2 - DNA, 2034 bp, Homo sapiens
gtcgacccacgcgtccggcaagatctctctggaccagctcgggtgcagggcctctgcgggagccctcctagacctctgcggcttctcctctaacatggccgactcggaaaaccaggggcctgcggagcctagccaggcggcggcagcggcggaggcagcggcagaggaggtaatggcggaaggcggtgcgcagggtggagactgtgacagcgcggctggtgaccctgacagcgcggctggtcagatggctgaggagccccagacccctgcagagaatgccccaaagccgaaaaatgactttatcgagagcctgcctaattcggtgaaatgccgagtcctggccctcaaaaagctgcagaagcgatgcgataagatagaagccaaatttgataaggaatttcaggctctggaaaaaaagtataatgacatctataagcccctactcgccaagatccaagagctcaccggcgagatggaggggtgtgcatggaccttggagggggaggaggaggaggaagaggagtacgaggatgacgaggaggagggggaagacgaggaggaggaggaggctgcggcagaggctgccgcgggggccaaacatgacgatgcccacgccgagatgcctgatgacgccaagaagtaaggggggcagagatggatgaagagaaagcccacgaagaaaaaagcctggttttgtttttcccagaatatcgatggacttaaaaaggctcaggtttttgaccaaaatacaatgtgaatttattctgacattcctaaaatagattaaattaaagcaattagatcctggccagctcgattcaaatttgactttcattttgaacataataaatatatcaaaaggtgttaaagaaaactgaattaaacccaaaattatgttttcatggtctcttctctgaggattgaggtttacaaagggtgttagcagatgcgaagtaaagaacgtcactttgaaacccattcatcacacagcatacgctacacatggaacacccaagccatgactgaacacgttctcagtgcttaattcttaaatttctttactcatgacatttcgcagtgcagagaaggcagaacccaagaaaaacgtcatctttgagactttgcttttgtaacgcagacatcagctttacacttcacaggagattgatggcattgaggaagattgcaatggagatcatgacactactgttaataaggccaggaaaactgccatttcaagttctgaaaaatgttttgagtatttgaatttagagaaacaacatggttccaagaaggagggtgtaaaacctgtaaaatactgtcaacatatgtattcattagttacaatctcatgtttgtgttttcttagtactgtctatttacaaacacgtaaaaaataccccaaatatgtttaagtattaaatcactttacctagcgttttagaaatattaatttacttgaagagatgtagaatgtagcaaattatgtaaagcatgtgtatccagcgttatgtactttgcgccttgtgacgtctttctgtcatgtagcttttagggtgtagctgtgaaaatcatcagaactcttcactgaagctaatgtttggaaaaaatatatacttgaagaaccaatccaagtgtgtgcccctacccccagctcagaagtagaaagggtttaagtttgcttgtattagctgtgccttcattattttgctatgtaaatgtgacatattaattataaaatggtgcataatcaaattttactgcttgaggacagatgcatacagtaaggatttttaggaagaatatatttaatgtaaagactcttagcttctgtgtgggttttgaattatgtgtgagccagtgatctataaagaaacataagcttaaagttgtttatcactgtggtgttaataaaacagtattttcaaaaaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagggcggccgc

SEQ ID NO: 3 - DNA, 1407 bp, Homo sapiens
gtcgacccacgcgtccggttggagcgagcatgtgggtctgcagtaccctgtggcgggtgcgaacccccgcccggcagtggcgggggctgctcccagcttctggctgtcacggacctgccgcctcctcctactccgcatccgccgagcctgcccgggtccgggcgcttgtctatgggcaccacggggatccagccaaggtcgtcgaactcaagaacctggagctagctgctgtgagaggatcagatgtccgtgtgaagatgctggcggcccctatcaatccatctgacataaatatgatccaaggaaactacggactccttcctgaactgcctgctgttggagggaacgaaggtgttgcacaggtggtagcggtgggcagcaatgtgaccgggctgaagccaggagactgggtgattccagcaaatgctggtttaggaacctggcggaccgaggctgtgttcagcgaggaagcactgatccaagttccgagtgacatccctcttcagagcgctgccaccctgggtgtcaatccctgcacagcctacaggatgttgatggatttcgagcaactgcagccaggggattctgtcatccagaatgcatccaacagcggagtggggcaagcggtcatccagatcgccgcagccctgggcctaagaaccatcaatgtggtccgagacagacctgatatccagaagctgagtgacagactgaagagtctgggggctgagcatgtcatcacagaagaggagctaagaaggcccgaaatgaaaaacttctttaaggacatgccccagccacggcttgctctcaactgtgttggtgggaaaagctccacagagctgctgcggcagttagcgcgtggaggaaccatggtaacctatggggggatggccaagcagcccgtcgtagcctctgtgagcctgctcatttttaaggatctcaaacttcgaggcttttggttgtcccagtggaagaaggatcacagtccagaccagttcaaggagctgatcctcacactgtgcgatctcatccgccgaggccagctcacagcccctgcctgctcccaggtcccgctgcaggactaccagtctgccttggaagcctccatgaagcccttcatatcttcaaagcagattctcaccatgtgatcatcccaaaagagctggagtgacatgggaggggaggcggatctgaggggctgggtgcaggcccctcagttggggctcccaccttccccagactactgttctcctcactgcctcttcctattaggaggatggtgaagccagccacggttttccccagggccagccttaaggtatctaataaagtctgaactctcccttccaaaaaaaaaaaaaaaaaaaaaaaaaaaaaaaaaaaaaaaaaaaaaagggcggccgc

SEQ ID NO: 4 - DNA, 1791 bp, Rattus norvegicus
gtcgacccacgcgtccggctgatcgaggctgaccttccaaacctagatggttgctggtcctgcacatgagtggaaatatattcatggagaaacttccatgatgcacagtatctgccgttcttcagtcctctgtctttctttgtcattcagttctgggcattgagcagccgcagtcacagctgcaggacctctctggaccagctcagtcgcagactgcgcaaccaccagaccactgcggcaaacaagcccagctgagccaagcaatagcgatggccgaccccgagaagcagggacccgctgagagccgcgccgaggacgaggtaatggagggcgctcagggtggcgaggatgcagcaaccggtgacagtgccactgcacccgcggccgaggagccccaggcccccgcggagaatgcgcccaagcccaaaaatgactttatcgagagcttgcccaatcccgtcaagtgccgggttctggcgctcaaaaagctgcagaagcgctgcgataagatcgaggcgaaatttgacaaggaattccaggctctggagaagaagtacaatgatatctacaagcccctactcgccaagatccaggaactcaccggagagatggagggctgcgcgtggaccctggagggagaggatgatgaagacgacgaggaagaagatgaggaggaggaagaagaggaggctgcagctggcgcaactgggggtcccgactctgccgagaagtgagcacagcagctgacagacttgagactgatgaaaggttgtcagttagatgggaattaaagtgcgtcacacgttgaaatccattcatcacactacaccttaacacccaagctaagacagaactcttctcaatgcttaattcttcagtttctttacatttcccagcgcagaggaagaggaacccaagaacgacgtcatctttaagacttttgcttttgcaaacccagacatcagctttacactccagaggagacaaggcatggaggaaggctggactgacagcatttactgtttatgtggctagaaaaactgccatttcaagttgtgaaaaatgttttgaatatttgaatttacagaaagaacacggttccaaaaataagggtgtattccatgtataatattgtcaacacgtgttcatctgtaatggtctcatgttatctgttttcttggtagtgtttgtttacaaaatcgtaaaaattaccccaaatgttttaagtattaaattcccttatagcattttagaaatataatttacttgaagagatgtagaatgtagcaattctgtaaagcatgtgtatccagtgttgcctagtttgactttgtgaagtctttttgtcttgtagcttttagcaagtagctgtgaaaaccatcagaactcctcaatgaagctaatgtttggaaaaaagtatatacttgaagaaccaacccaagtgtgtatccccaaccccagctcagaaataggaaggatttaagtttgcttgtattagctgtgccttcattattttgctatgtaaatgtgacttattaaatggtgcataatcaaattttattgcttgaggacaaaaatggcataaagggaagacttttgggaaaaagacatttaatgtaaaggcctttagcttctttgtgggttttgaactatctgtgaatcaatgttctgtaaagaaacacaaacgtaaagttgtttaccactgtggtgttaacaaaacagtattttcaaaaataaaaaaaacttgttattctgaaaaaaaaaaaaaaaaaaaaaaaaaagggcggccgc

SEQ ID NO: 5 - DNA, 1057 bp, Rattus norvegicus
agcggacaggaccagtgaagaagccacggtagctgctgccatctgctgccggagccggccttcggcaaaggcctcctgggttcaccagtgacagcctcaggcaggcattgtacctgtggctggacgcagagatggacgtcctggctctcttgtgtctagccaaaagtggggagactctgcctgggggaacttggcgtctcatcctgggtacccattcctggtgtatgtgtggggaagcacctccttcatggtcagggggcctgtgcttggccttctgccatcgaagatgttaagctatagttggctttggccagctgctccagtatatcagaacctgagagcacttgctacaaggctagtgttcaggccttaggcctccagagtgaatgtatcctgcaggaagataatgatggatcgtgacccttgacggtcacccccctcccccaggtcagatgtcaccagactagaacagtatctgaaagctgctggggccactcacagcttgcttactctggagacagcattttgggctccctgattaatgcagatcagttctgcccacctccaggggtggatccagctgtgaggctcacctgtatcttccagatgttctcatctgctgcaccgaaggctctggccctgctcaggagaacacgctacgaactcctagctgactctgtttgcactggagaaccacacagggcttaccccactaccctgtgcactgactggcttcactttatggaggaagagacagggccagagaagcaatgtcatgcagccagtgatgctaggacataaatccagagtggctggccctgaagccatgcctcttggcaatgccaggctgggcatcctatttttgaagcaaacaaaaaatgagaggacaggctgtgcttcagcggcttgttcctggacctatgctcccttagccccagtcccacggattatgtggagagtggaggagcaacagagggcgactgtactaaggccacacaagtcgacaagaacacctatatccttttgacctcttctgcttttttatagtaagctttcc

SEQ ID NO: 6 - DNA, 2250 bp, Rattus norvegicus
cactagcctagagcctgaaggtatttctccatatggacatgatgtctcaccctccaaccatggattctaaaccctgccagatgttccagccttgatctctttgctacttacccctatatctggaggattcagtgggagagccttgcaaggtaataccaggctctccctgtgaccagctgaggactcatctgcccccacagtgccaacaaggaccccccctctggaaaggaggtcagattgcagttccaggtattggggaggctgatcagctgtctcccaggtggcaagaagtttagtgaagctaaggtactccatcttggggaccctgttggagtagctcactgactagaaaacccttaagaacctccactgcctactgaatctcatcccttctcatcagccctcaatcaggactcttcacttcttgggaagcttctctgggccagaagtgagcatggcctctgtcctgcactgtcctgttccattcatactcactggtttgactagacattctccaaaagcagaacggagatggatgcgagagagaagcaggaggagaccttaatgtcagctttggagcatccccaaattccaagaaggctcccctgctagtgaacagtagtcaccccttcccactgtcttggactttggtaaatttaccccagagtggccagcatttgatccagacacagactaaaggattgattgttacccgaagtcatgtcactgggtagcagcagggtctgctcctgactcatgctgccactacagctccctgtctcctcctgactgtctgcttcagggcccctggcccctggctgccttgatccttggcttctggcactcacctactctcttttattgagcatctctgggtgagccctccctcccctctgggcctccacctccagaaagagttgtaatctgagtaggccctggagttcctcatttcgtttgagctcccgatgcctactagcaatgggccgaccagatcacaagcagctgaagcttggtcttcaagggcatgccttttccatggccgagaggacagacaggcttcaccagaaggggtactgagggagagaagaatgtaaacagaatctagttaagacaggaacacagaattgctcttgtggggttggttgtccatgatcttgaaggttctctaggtcaattccccagtttctaaagactaggcctctctagggtaccaggaagactcaagaccagtaagtaaggttgattgatggcatgcgttcctgattggcagcagagtgctctcctgactccttcagccactataactgccctcttcctcctaatcctcctgactgactgactgcttcagggccattggctttttccaccagagcacgactctgtcctgaggctttatctcacgtgacactaggcaacattagcaagtttacctaccgaacacctgcactgggaatggtgttataaggaagagagagaggtgtgaaagagaacacctctctgcttctgctgggaagcaggcattagtgggacagtgtcactactgagctcaggtacccagcatgaagtgaccaggaagatctctggagagggatggtttgagctgggcttcagaagatcaatcatatcagacaggaggggcacaggctcgactctaaaggtcccctaaggaccgctctttcaaagaactcctgtgtctaaaactcctgctccaatgggtgtgagcttgaggggggccaccagctcaacttccttttctagtgcagctctccgggtcccaagatcgtggcatggagtccactaagctaggccattttatatatgagcatcagaacggaatttttcagtgccaaacaatctgaggcaaagccagaggcaggccagaggacatttttgttttattttgttgagttctcgtgttatcaagacctacctcccaccccaagtagcccaggacaagtggagcagaaactcagatgagaacataagaatgtgaaaaacagtatccatggagaactagttccagcccacccctcaccctccagcataccaaaatcttgtgtagaatggtgtagtatttgcatataatgatgcatattctcctatacactttaaatggtctctagagtacgtataatgcctaatatgatgtaaacgctatgtcagcccttacacagtgttgttgtaataatgacaagaaaaataaaaaaaaaaaaaaaaaaaaaaaaaaaaaaaaaaaaaaggggcggccgc

SEQ ID NO: 7 - DNA, 2046 bp, Rattus norvegicus
gtcgacccacgcgtccggtttttattactttaattattgttataaaaagcctgccatttttaatatgtggtttggggaatttttgtttgtttttcctgtttgggggtttcctttgttttttgttttttttctggatttaaaaaaaaaaaaaacaaaaccttgcttttagtgtttgtactgctgctggtcagaatgttaaaacgctgaagttctaggaaataggagagctcgcctgtgcagcattccacacagcagggctaagggggcacctaggtctggtcagctgtccagggcatggtgacccatgagcagcaggaacttggcacagctctggcagctgagctcctgagacaggcacagctctggcagagagctccacactgggggatctcccttcccagtttcaagtcctcagtcagggctgaccaacttgaaagagatcctcttcctgccagagcctgtgactatcctccatcacggggggggggagaggaggcagagcctacccttggccaccaggctcaatggctgtacagagcagctgccttgcagtctgtccccaccctgctctacccccaaccccttgctctgcctgccaagagtcttctagacaaggaagtgccaccagtactgtcagcagtcaacaaagcaccttcctctgcctacagccagtcagagatggtccaaaggagagcagaggctgcacaccctgggcaaagcactgcccagttttccagttaagtgctgcgtgcgctcagtgttcctttcccaggctaagaacacaccgatgactggaagcttttgctaatctgcttggcaatggcttctgggaaaggtaggacccataacttaagacatgcacagtctctcccaccgtcccacaggagttcccctggctgagtatacgatccaaagcaagccatgccctcccaggtcagtctggggcacaagctgagccgatgactagcaatgcctatggcctttcccttgcctgccctcctccagcatctccgcctgtggagaccgagtacccccgtgctcatacgtaaagtgacaatcagaaccaggtacaagccaggaaagtggcagctgactgccactcagaccacgtggcgcttttcccatcccacggtctcagagctggacgaggctaaatagaacacagtagcccccccttccaggtactgcaccgtccctggagatccctctgacccttccctgctacagatctgctctgctctaggctggactgtggaattagcatgtacatggaaatcccagtccttgaccatggcttcccactccacctgcaagtgatagatgccatctgtcctgggtgtctgatcagacccgccaccatcacagatgagtgaccaagaggggggctgtcaacacctcggtacatggtgatcttaaaaccacccaactgcaccatcgcaccagactgtacctctggggcacccagaacaagccccaccctaacagtgggggccacagccaggcttccagcactgagtctcaaccagctaagttgaatggcaaactcgatgcctccgcccccacccctcagctgcccaggccccagcatgcagatggcctgcacagcaggctcagcacctctgaggtgtgcattagccacttaacagcagcagtctgtactcaagtacaaaagcttttacttcacgacttgccgtagcctgtccccactgtctgatccagtgcttaacttcaaccctagagtctgccttgaccctgaggaggcatctcactggtttcgtacttgtgtgtgccctatgcctcactgctggggccgcgcacccagacccagccaggagggaggatgggtgcctcggtcgctctgggggcagtttagatgctgtgaaattaaacccgttctaagtgtacttgtttgaattaactgtattgtaatattatttgttgaatgtagtaattaggtatttatgaatatattgctgtaatttctgacatcccaaaaataaaatcttcctaaatcatgttaaaaaaaaaaaaaaaaagggcggccgc

SEQ ID NO: 8 - DNA, 988 bp, Rattus norvegicus
gttttagcacaggctttttgaaccctctacctgactcagcattttttattggtgaaaaaaattccaggatgaagagccctgttttagaatgcaaataaagtaagaggcttattttttaatgttaggcaattttgaaatcttatgcctttctgcatgcatgacagtggaatgggcaactacaaattccatattgccattaaaatatattggattatattagcattcacagattacttctagttaatgctgggatttcatttttgaataatggcaccttccatttgtacctccattttctgaagtactttggaacatattttcattttagaatatagttcttaagaattttctacaaaattagtgaagaaacatagagaatgctataaaaaggtgggtaggtgggttggttggttggttcattggttcatatgactaaagagagtctctagttttatctgttgtactgtcatgctgaatacgttatcttttcagatagtttttaagagtatgtcttaggagcaatttgaggaatgaaagtctagaatcattttattcagttggttaataacttagtaagcattgaatttctgttggcattcatattttttcaggaaggaatattccaaatcacttatccaaatactgatccagatatttaaccacaaatattttaaatagttattttgtgaaagtccagaaagtccagcagaatgaaataaggaggtaacacttttgtgaacaaaaattctcagccaaccttaaaggaacaaaactacatgcaacttttcttactctgttctagtttgtcttactgacttatcatttgtgtgattttgttaaggataatttttgtcaagatgaatgtgttgtcttacatctatataagagaaatttatgtaatccacatttgaataaatacatcaagattaaaaaaaaaaaaccaaaaaaaaaaaaaaaaaaaaaaaaaaaaaaaaaaaagggcggccgc

SEQ ID NO: 9 - DNA, 974 bp, Rattus norvegicus
tgctgggccagggtgaggaggggccgagctgcgagcttgggcgctgcagcctgggcctgcacgtctctggctctgagcgaagccactgggaggagatgctaaaaaacccacgccggcagatcgccatgtggcaccaattgcacctgtagtccacatgcctacacccaaagacagatgcaaacatgttggctgaggccaggaactcattcctctcttactgtattcccaatactaaagagtgagcagcattggcaggacagtgagcaggactggcatgtcagggtcactcagaagactgtgtgtcttgctcatctgtgttttggaaaaagatgtgcgtcaggtatactcagtagacagtccctgctcacattggtctaaagcagcagttctcaacctgtgtgtcatggcccctttggccaccctctatctccaaaatatttacattgtgattcataacagtagcaaaattacagatatgaagtagcaatgaaaatagttctgtggttggagattgccaaaacacaagaaactttattatagggtcacggcatttggaaggttgagaactactggctggcagcaggtccctgggccacgggggctcatgccacactgatgctctaggtggaatacaccaggctcctgtccttaactagcacaggggttctggagcaggaggggctgcgcttgatgaggtcccttcgacactacccaggcaacctttccacccttgacctccagaatctcaacactgggcagtatgaagacaagagctctccgcttacttctccttttttacatttctgctgttcatatcccactcttgaactgtactgtgtgttttgactgttttatttaaggaattgatgtgggttttgtttgtctttgatcacacgtagagtgccctttccctggcagacctaggttgccgttcctctggagtgtctgtggcattctgaggacaactgtcatgt

SEQ ID NO: 10 - DNA, 637 bp, Homo sapiens
acagaaatgcatctgggtgagcatgtgcttatttgtattattctgaagctcctgagagtccctcaaagacccatgctgggcctccccgtcaccgagattgcctccgagaacttctctcacagaagcaggaagaagagcacttcactcctgcccacaggagcagcaactctgaattccgcagccaagggctctttctggcttgactcccagcaaactgaaggttccagggaagaagtgtggattaattctctaaagagcctgcctttgtgtaagaccttctagatggtctgccacttgctgagtgggggcatcacagccttatggtgagatgggttcaaaagaacaaggttcactgcgtggcttggcaaacacctgtgatctgagtccatctgagacaggaatgaatcagataatgagaccctatctgaaagcaatcaataaaaggccaaggctgtattgggtggtgtctggcccccaatggcagtcatctattactaggttttgcctctgacctggaatttttgtgactgctggataagaccctaaaatatatgctgggctgttggacctcttgctcaccccttccaacctcgaggctaggaatgggcgggtacctgcttccaaaagaaagtgtctc

SEQ ID NO: 11 - DNA, 875 bp, Homo sapiens
  misc_feature (1)..(875): n = A, T, C, or G
gggggggcggatcgaccacgcgtccgcgaagtcaccagtatggattccggaccgcctcatcacatcatacaatcccagggcamgaaaagcagaaagtctcccctcaacaaccgacagcgaaagatgagagcggagcgtgcctcattcctgatgacgccagacagctctgagcagcgcctcacttctgctttcaccaggcttcagctgcatggtcgctcatctaccacgctgttgccaacttgggatcagcttcaagaattggtctctgctgcaagagatagcgtgcttcctctccgactcccagttactccctccaatcttctcctgctatgctttcacttctcttcacccagggacatggttttttgatcctggcatagaggccattcatcatgcctcctgtgtacttcgtagaggttcctggctgtcacatttctgcacgatggtgaacaagtttgtttttctgattctcataacaagaaaaggttaagactaaactatctaatcttcaaccccatccaagatctgaggaggagaaatgttgcctgaataaaaggaaattcaaagcaaacagcttccaaacctaagaaatgacagaaattgttggctacactgggagtcagtcagcaaatattcctgtgtcactgtggcatccaccttcaactcactgggccaggagctctgacaacaatcctttgaagcaggtcacttgaaggactatcctattttttttccaaaaactgggacacanacttccccatgatcaacttctgcacagtctggacagcttnctggcagcagtagaggttggccctgcagctttgggcanggcccatggacaaagagattcgcacctgatgggaacaatcttaaaa

SEQ ID NO: 12 - DNA, 2792 bp, Rattus norvegicus
gtcactacaacactggtgacatcacagaggatgccagaactctctaggagacaatagaatgtccaagaagggacagctgatgtcatggggagcagactttgcctccctgctaatgttctccctgtactgagcataagcagaggtgccctttccaggagactttcctggggcagagccactgagcatctcctgcttccaaagccaaattttcctcaaggcttgattataaaaaccttagtaattcctatgcatctaaatgaatgtttccttccatatcttgccccaaaatgtctcactaactcaaattgtcctcattcaccaatgttagccattaaaacttcctgagatttcacaccagcttgaatatgcagtcaaaagttctcttgtctcattatgtacaggtgctttaaatcacatcaagaaatagtctgcatggtaggttacaacatgcgtgtgtgttaggggaacactcatgaagtcatgtgcttagcaatcgacaattgccagaaaattccttaggagaaagagttgaagtctttaaggtgctaggaaaagtgtggagaccagtttgcagaggaaaatgcaatattagagccaccagtgaagggaacctcatgctgcttgtggggttctgctctctgcaccaatgttgctaaaggagcactgctcacaggcctgagtaacatgtactatgagcttttatcagaaaaataaaatacacaagagatatagagggtgcatagaaacgtctaaaatgaggctataaacatcgataagtggatagagcaaggtcccagcacctgtacactgggaaatggccaccccaccaaacacaaaagcatctatcatagtaaaaaatgaatactttatagaatgcacccactaacactgattgatcacatccatagttcaaattttgggggcaaattcatggccttaactatctttgtctcaccttatatagcaaaaaataagaaagaacgagaaaagaaaaagtcaaaactaaaagctcaagcttctcatatgaggattgcaggaagtgttatatgggagtcagttctgattaggccattttacatagaacagtgcatagtgacccttaatgtgatgggccctatataaagctttttgcaatattggaattttaacaaacctcatccagaacgtacggaagaggcaccatatgatcaccatgtgatcaccatgtccttactctgtatcaggttatttttctgcatccttgattgtcaggcatataacagcaacaacctgaaatagctggtagacatgtaaaaacatggccaaaactatagttgtgggtaacacacctcaactgtgaaaggctaatgctgtctgcacagtccttggaggccactcttttaagcctatttattgcaaagtgaaaggaggtttatctgagctcaaagtgatcctggccagccaggtagagggcaacatgtgatacttgagaccctgttttaaaccagcacagaaatccacacttcattcaaagcatcatctacgaattctcgattatttaccattctctccctaccagtcgtactgtttggaatggtagaaaacaattggaactgatctttctattttaaggaccttgatctctctttttttaggtccacattaagagtgcatacaaatggacatcaccatctgaacatcttagtattatctactctgtatatatagcataatactgtgcctatgaaaatctcaagcttccttgagtgtagttgccccatcaagaagtgtgaagttctgagttcaaaccccagtgatgctttaaagggactagaagaaagccatcagagatactccacttctttgtccttagattaaatcaagttgacacaatgtgaacctggtgggctatttaaccatgcatctttctcccaaccccacttccacgagttcagtccatggcagaattcatcccccaccatcctagatatttctttgtatatttctacaagagccaggtttctttttttttaatagaattagttgtagtcaatcaactgctgccctatgtgagaaatgattagtatctattgcaacttaccacagtgtatttgtgtatacaagtacattattggaaatccagcattactgagggattggaaaccagtgctgtggccttgcctttctgtgcctcttcctggctcacaggcagcaggcggcagaccttgagttcctcttcagggacagagtgatgttgaatgggcactgagcagtcagagaaacacagctgaggcagctcctttcagttatcaaacacatcatctccttcccatcttaagggctactgctgaggtcttgcccccatacctaccttaatagcagcgaatttgttacacagcagccagctctttcctattgttctgtaatacgcctccacatcatggatgccaagaaaaacactaacaaagaacaaaaaactctgacgttggactgggtggtgctggtcacataccctgttatgttctgtatgaggtttgttaatcttaacaagttccccaagtgtattcttcacataggacccaatagattcaaggaaaataggaacaattatgtgtaaaatggcctgagtcagggcccatagaccgaagcatttccagcacttgtgcatgagcccatgtggattttgcaatgagctgggcctagcaatgtccagtacacagttgtgagctgttgctctggttgaactgtattggtgtgtgcattaataaactacacctatttaaatgaaaaaaaaaaaaaaaaagggcggccgc

SEQ ID NO: 13 - DNA, 617 bp, Rattus norvegicus
gaattcggcttggaaagctggtaccgcctgcaggtaccggtccggaattcccgggtcgacccacgcgtccggggttccctggctccctttgttcaaccaagaggaaatctgagtcaagccagttgagctatcaagagcctaggccacacctctccatgctccccgaacccaacacacccagaaacctgtgatttcttttctcctgttttgacaagggaccacatatcaacacaaaattataatctccacaaggaatacaaatgtacagacgaattcggcttggaaagctggtacgcctgcaggtaccggtccggctcccagaacaaagcattcgtccgtggaaattcaattccttttcagatgctccagttgttgtggcccattgctagaaaggtcagcttaagcccgcagccccccggtaggaagctcaaaatacactgcccagaacttactcaacattccccagaagtgctgtctttgtccatcattactttgagacgaaaggctagactccttcactttagtctctccagaatcgacattccccctcccaaaacgcttgcgaagacgggaaaacatgtcttagcttgtaaccgccagactcagactgagagaaa

SEQ ID NO: 14 - DNA, 1475 bp, Rattus norvegicus
gtcgacccacgcgtccgcccacgcgtccgcccacgcgtccgcccacgcgtccggttggtcagccggcgactgacaggggcgcgagcccgggcacctctgcttgcgagtctcctcgaggcttggtgccgccagggcaggaccacctcctcctactccgctttctcggagccgtcgcgtgtgcgggcgctggtctatggcaaccacggggatccagccaaggtcatccactataaaagcagaacaatagtctgcaagctattgaaatgggatggaagctggccggcctataaagcacttggcagggagcctggctgaacactcactgactgaagaacctggagctcactgctgtggaaggatctgacgtccatgtgaagatgctggcagcccctatcaatccatctgacataaatatgatccaagggaactatggcctccttcccaagctgcctgctgttggagggaatgaaggtgttggacaggtgatagcagtgggcagcagtgtgtctggattgaagccaggagattgggtgatcccagcaaatgctggtttgggaacctggcggactgaggcggtgttcagtgaggaagcgctgattggagtccctaaggacatccctctccagagtgctgccaccctaggtgtcaacccctgcacagcctacaggatgttggtggactttgaacagctacaaccaggggactctgtcatccagaatgcgtccaacagtggagtagggcaagcagtcattcagatcgcctcagcccttggcctaaagaccatcaacgtgatccgagacagacctgacatcaagaagctaactgacagactgaaggatctaggagctgattatgtcctcacagaggaggagataaggatgcccgagaccaaaaacatcttcaaggacctgccgctgccccgactggctctcaactgtgtcggtgggaagagttccacagagctgctccggcacctagcgcccggaggaaccatggtgacctatggaggaatggccaagcagcctgtaacagcctctgtgagtatgctcatttttaaggacctcaaacttcgtggcttttggttgtcccagtggaagaagaaccatagtccagatgagttcaaggagctgattctcattctctgcaacctcatccgccaaggccagctcacagcccctgcctggtccgggattccactgcaggactaccagcaggctttggaagcctccatgaagccttttgtgtcttcgaagcagattctcactatgtgattactccagaggaccaggaggaaagcaggagaggcaggccagcaagattggctggctgctggccctccatgaggactccagactgcctcaccctcactgcctcttcctaccaggagggtgggaggccaaccccagggtccctaataaaccctggacttcccaagtaaaaaaaaaaaaaaaaaaaaaaaaaaaaaaaaaaaaaaa

SEQ ID NO: 15 - DNA, 2738 bp, Homo sapiens
gtcgacccacgcgtccggagatatccttaataagcgacaatgagttcaagtgcaggcattcacagccggagtgtggttatggcttgcagcctgatcgttggacagagtacagcatacagacgatggaaccagataacctggaactaatctttgattttttcgaagaagatctcagtgagcacgtagttcagggtgatgcccttcctggacatgtgggtacagcttgtctcttatcatccaccattgctgagagtggaaagagtgctggaattcttactcttcccatcatgagcagaaattcccggaaaacaataggcaaagtgagagttgactatataattattaagccattaccaggatacagttgtgacatgaaatcttcattttccaagtattggaagccaagaataccattggatgttggccatcgaggtgcaggaaactctacaacaactgcccagctggctaaagttcaagaaaatactattgcttctttaagaaatgctgctagtcatggtgcagcctttgtagaatttgacgtacacctttcaaaggactttgtgcccgtggtatatcatgatcttacctgttgtttgactatgaaaaagaaatttgatgctgatccagttgaattatttgaaattccagtaaaagaattaacatttgaccaactccagttgttaaagctcactcatgtgactgcactgaaatctaaggatcggaaagaatctgtggttcaggaggaaaattccttttcagaaaatcagccatttccttctcttaagatggttttagagtctttgccagaagatgtagggtttaacattgaaataaaatggatctgccagcaaagggatggaatgtgggatggtaacttatcaacatattttgacatgaatctgtttttggatataattttaaaaactgttttagaaaattctgggaagaggagaatagtgttttcttcatttgatgcagatatttgcacaatggttcggcaaaagcagaacaaatatccgatactatttttaactcaaggaaaatctgagatttatcctgaactcatggacctcagatctcggacaacccccattgcaatgagctttgcacagtttgaaaatctactggggataaatgtacatactgaagacttgctcagaaacccatcctatattcaagaggcaaaagctaagggactagtcatattctgctggggtgatgataccaatgatcctgaaaacagaaggaaattgaaggaacttggagttaatggtctaatttatgataggatatatgattggatgcctgaacaaccaaatatattccaagtggagcaattggaacgcctgaagcaggaattgccagagcttaagagctgtttgtgtcccactgttagccgctttgttccctcatctttgtgtggggagtctgatatccatgtggatgccaacggcattgataacgtggagaatgcttagtttttattgcacagaggtcattttgggggcgtgcaccgctgttctgggtattcatttttcatcactgagcattgttgatctatgccttttgggcttctcagttcaatgaagcaataatgaagtatttaactctttcactacagttcttgcaagtatgctatttaaattacttggccaggtataattgccagtcagtctctttatagtgagaaaatttattggttagtaatataaatattttaaactaaatatataaatctataatgttaaacatatgttcattaaaagcatagcactttgaaattaactatataaatagctcatatttacacttacagcttttcatttgatcaggtctgaaatctttagcacttaaggaaaatgactatgcataattatacctgaccatgaaaaaaataagtacctcaaatgcatgcatttgcactggtgattccaactgcacaaatctttgtgccatcttgtatataggtattttttacatgggttgacatgcacacaacaccattttcattcagtatgaaccttgaggctgctgccatttttccacttaaccaaaccagcctgaaggtgaacctcgaaacttgtttcataaatctttcaaaagttgttttacatcaatgttaaaatttcaaaatgctgcagggtaatttaatgtataaaatattagtaagaaaaagtatgtattgcatacttagtagaatagatcacaacatacaaattcaattcagtgcatgctttaggtgttaagcatgagattgtacatgtttactgttaggtccttgcatctgtggtgctaggtgagtatgagaagatgtcaaggactggacgtattttgttgcctaaaaaaaaaaggctgtttgtaggcgttttaaatatgcttattttgtgtgtctctcactacctattacacactgttgctttgtgggtttgttttgtatgtgcgtgtgttatacagtagttaaatttccatgcagaaaaataaatgtcctgaattctcatattagtattctttattgtatatcatgcatgtaatttatttagaaatgtaggtcttactaaatgtatatgcatgtatttcagattatactaggatttcttggattagaagcagattgtgttaactgtaacttaaagaatgaatgttaaataaaatgatacagatttattttcttcattacaaaaaaaaaaaaaaaaaaaaaaaaaaaaaaaaaaaaaaaaaaaaaaaaaaagggcggccgc

SEQ ID NO: 16 - DNA, 1664 bp, Homo sapiens
gcggccgcagccccggccgagcaggcgccgcgggccaagggccgcccgagacggtccccagagagccaccggaggagcagctcacctgagagacggagccccggctcgcccgtgtgcagagcggacaaggcaaaatctcagcaagttcggacctctagtacaataaggcgaacctcctctttggatacaataacaggaccttacctcacaggacagtggccacgggatcctcatgttcactacccttcatgcatgaaagacaaagctactcagacacctagctgttgggcagaagagggtgcagaaaagaggtcacatcagcgttctgcgtcatgggggagtgctgatcaactaaaagagatcgccaaactgaggcagcaactacaacgcagtaaacagagtagtcgtcacagtaaggagaaagatcgccagtcacctcttcatggcaaccatataacaatcagtcacactcaggctactggatcaaggtcagttcctatgccactgtcaaatatatcagtgccaaaatcatctgtttcgcgtgtgccctgcaatgtagaaggaataagtcctgaattagaaaaggtattcattaaagaaaataatgggaaggaagaagtatccaagccgttggacataccagatggtcgaagagctccacttcctgctcattaccggagcagtagtactcgcagcattgacactcagactccttctgtccaggagcgcagcagtagctgcagcagtcattcaccctgtgtctcccctttttgtcccccggaatcccaggatggtagcccttgctcaacagaagatttgctctatgatcgtgataaaggtctcgtcagcctatctcggcccctctcttttcatgtcctgacaaaaacaaggttaatttcatcccaaccggatcagctttctgtcctgtaaaacttctaggccccctcttacctgcttctgaccttatgctcaagaactctcctaactctggccagagctcagctttggcaactctgaccgttgagcagctctcatcccgggtttcctttacgtctctttctgatgacaccagcacagcgggctccatggaggcctctgtccagcagccatcccagcagcagcagctcctgcaggaactgcagggtgaggaccacatctctgctcagaactatgtgatcatctaaaaaagggggagctggcctccaccctgtgttccatggattcggaacaagatttcagacatctgcatgagtgacaaactttctgaacaccaccaccaccaataatacttatcagcatcataaagtatctcttaaacactgatcttggcagggacggaactcctattcagcagtttttgtggaaagcagtaatgcttgcaaaacgtgtgtgtcattcagcattttaagtggagactatgcatttcatagtatatttgacagattagtactgtgtcctgtgttttgttccagattcttcagtataaataagctctatatcaaaaagttgcctgtctaaatagaaaatgtcttgctgtgttttgtcctatggaaaatactgtaattcaggattatgtttacaattgatccaggtgtttgtttctaacttctgtaatacatacaatgcaaaaaaaaaaaaaaaaaacggacgcgtgggtcgactcc

SEQ ID NO: 17 - DNA, 3206 bp, Homo sapiens
gtcgacccacgcgtccgggcgaggcacggacggcgggcgcccggtacctctgcccgcggtcctcgctctcgggcggggcggcggcgacgcggacctgcggactagcgaacccggagcacgacatcataaaataaatccatcagaatgacaccttctcaggttgcctttgaaataagaggaactcttttaccaggagaagtttttgcgatatgtggaagctgtgatgctttgggaaactggaatcctcaaaatgctgtggctcttcttccagagaatgacacaggtgaaagcatgctatggaaagcaaccattgtactcagtagaggagtatcagttcagtatcgctacttcaaagggtactttttagaaccaaagactatcggtggtccatgtcaagtgatagttcacaagtgggagactcatctacaaccacgatcaataacccctttagaaagcgaaattattattgacgatggacaatttggaatccacaatggtgttgaaactctggattctggatggctgacatgtcagactgaaataagattacgtttgcattattctgaaaaacctcctgtgtcaataaccaagaaaaaattaaaaaaatctagatttagggtgaagctgacactagaaggcctggaggaagatgacgatgatagggtatctcccactgtactccacaaaatgtccaatagcttggagatatccttaataagcgacaatgagttcaagtgcaggcattcacagccggagtgtggttatggcttgcagcctgatcgttggacagagtacagcatacagacgatggaaccagataacctggaactaatctttgattttttcgaagaagatctcagtgagcacgtagttcagggtgatgcccttcctggacatgtgggtacagcttgtctcttatcatccaccattgctgagagtggaaagagtgctggaattcttactcttcccatcatgagcagaaattcccggaaaacaataggcaaagtgagagttgactatataattattaagccattaccaggatacagttgtgacatgaaatcttcattttccaagtattggaagccaagaataccattggatgttggccatcgaggtgcaggaaactctacaacaactgcccagctggctaaagttcaagaaaatactattgcttctttaagaaatgctgctagtcatggtgcagcctttgtagaatttgacgtacacctttcaaaggactttgtgcccgtggtatatcatgatcttacctgttgtttgactatgaaaaagaaatttgatgctgatccagttgaattatttgaaattccagtaaaagaattaacatttgaccaactccagttgttaaagctcactcatgtgactgcactgaaatctaaggatcggaaagaatctgtggttcaggaggaaaattccttttcagaaaatcagccatttccttctcttaagatggttttagagtctttgccagaagatgtagggtttaacattgaaataaaatggatctgccagcaaagggatggaatgtgggatggtaacttatcaacatattttgacatgaatctgtttttggatataattttaaaaactgttttagaaaattctgggaagaggagaatagtgttttcttcatttgatgcagatatttgcacaatggttcggcaaaagcagaacaaatatccgatactatttttaactcaaggaaaatctgagatttatcctgaactcatggacctcagatctcggacaacccccattgcaatgagctttgcacagtttgaaaatctactggggataaatgtacatactgaagacttgctcagaaacccatcctatattcaagaggcaaaagctaagggactagtcatattctgctggggtgatgataccaatgatcctgaaaacagaaggaaattgaaggaacttggagttaatggtctaatttatgataggatatatgattggatgcctgaacaaccaaatatattccaagtggagcaattggaacgcctgaagcaggaattgccagagcttaagagctgtttgtgtcccactgttagccgctttgttccctcatctttgtgtggggagtctgatatccatgtggatgccaacggcattgataacgtggagaatgcttagtttttattgcacagaggtcattttgggggcgtgcaccgctgttctgggtattcatttttcatcactgagcattgttgatctatgccttttgggcttctcagttcaatgaagcaataatgaagtatttaactctttcactacagttcttgcaagtatgctatttaaattacttggccaggtataattgccagtcagtctctttatagtgagaaaatttattggttagtaatataaatattttaaactaaatatataaatctataatgttaaacatatgttcattaaaagcatagcactttgaaattaactatataaatagctcatatttacacttacagcttttcatttgatcaggtctgaaatctttagcacttaaggaaaatgactatgcataattatacctgaccatgaaaaaaataagtacctcaaatgcatgcatttgcactggtgattccaactgcacaaatctttgtgccatcttgtatataggtattttttacatgggttgacatgcacacaacaccattttcattcagtatgaaccttgaggctgctgccatttttccacttaaccaaaccagcctgaaggtgaacctcgaaacttgtttcataaatctttcaaaagttgttttacatcaatgttaaaatttcaaaatgctgcagggtaatttaatgtataaaatattagtaagaaaaagtatgtattgcatacttagtagaatagatcacaacatacaaattcaattcagtgcatgctttaggtgttaagcatgagattgtacatgtttactgttaggtccttgcatctgtggtgctaggtgagtatgagaagatgtcaaggactggacgtattttgttgcctaaaaaaaaaaggctgtttgtaggcgttttaaatatgcttattttgtgtgtctctcactacctattacacactgttgctttgtgggtttgttttgtatgtgcgtgtgttatacagtagttaaatttccatgcagaaaaataaatgtcctgaattctcaaaaaaaaaaaaaaagggcggccgc

SEQ ID NO: 18 - DNA, 1175 bp, Homo sapiens
gcggccgcctgctggccggagcctatcacgccgtagtgctgcgagagcgcgccgctcagtgcctgcttctggattgtcgctccttcttcgccttcaacgccggccacatcgtgggctcagtgaacgtgcgcttcagccaccatctgccttgcttacctcatgaggactaaccgagtgaagctggacgaggcctttgagttcgtgaagcagaggcggagtattatctcccccaacttcagcttcatgggccagctgctgcaatttgagtcccaagtactggcccctcactgttctgcagaagctgggagcccggccatggctgtccttgaccggggcacctctactacaacggtcttcaacttccctgtctccatccctgttcaccccacgaacagtgccctgaactaccttcaaagccccatcacaacctctccgagctgctgaagggccaggggaggtgtagagtttcatgtgccaccgggacgacactcctcccatgggaggagcaatgcaataactctgggagaggctcatgtgagctggtccttatttatttaacacccccccccaacacctcccgagttccactgagttcccaagcagtcataacaatgacttgaccgcaagacatttgctgaactcagcccgttcgggaccaatatattgtgggtacatcgagcccctctgacaaaacagggcagaagggaaaggactctgtttgagccagtttcttcccttgcctgttttttctagaaacttcgtgcttgacatacctaccagtattaaccattcccgatgacatacgcgtatgagagttttaccttatttatttttgtgtgggtgggtggtctgccctcacaaatgtcattgtctactcatagaagaacgaaatacctcactttttgtgtttgcgtactgtactatcttgtaaatagacccagagcaggctttcagcactgatggacgaagccagtgttggttgtttgtagcttttagctatcaacagttgtatgtttgtttatttatgatctgaagtaatatatttcttcttctgagaagacattttgttactaggatgactttttttttatacagcagaataaattatgacatttctattgaaaaaaaaaaaaaaaaaaaaaaaaacccacgcgtccgcggacgcgtgggtcgac

SEQ ID NO: 19 - DNA, 1172 bp, Rattus norvegicus
gcggccgcctgctggccggagcctatcacgccgtagtgctgcgagagcgcgccgctcagtgcctgcttctggattgtcgctccttcttcgccttcaacgccggccacatcgtgggctcagtgaacgtgcgcttcagccaccatctgccttgcttacctcatgaggactaaccgagtgaagctggacgaggcctttgagttcgtgaagcagaggcggagtatctcccccaacttcagcttcatgggccagctgctgcaatttgagtcccaagtactggcccctcactgttctgcagaagctgggagcccggccatggctgtccttgaccggggcacctctactacaacggtcttcaacttccctgtctccatccctgttcaccccacgaacagtgccctgaactaccttcaaagccccatcacaacctctccgagctgctgaagggccaggggaggtgtagagtttcatgtgccaccgggacgacactcctcccatgggaggagcaatgcaataactctgggagaggctcatgtgagctggtccttatttatttaacacccccccccaacacctcccgagttccactgagttcccaagcagtcataacaatgacttgaccgcaagacatttgctgaactcagcccgttcgggaccaatatattgtgggtacatcgagcccctctgacaaaacagggcagaagggaaaggactctgtttgagccagtttcttcccttgcctgttttttctagaaacttcgtgcttgacatacctaccagtattaaccattcccgatgacatacgcgtatgagagttttaccttatttatttttgtgtgggtgggtggtctgccctcacaaatgtcattgtctactcatagaagaacgaaatacctcactttttgtgtttgcgtactgtactatcttgtaaatagacccagagcaggctttcagcactgatggacgaagccagtgttggttgtttgtagcttttagctatcaacagttgtatgtttgtttatttatgatctgaagtaatatatttcttcttctgagaagacattttgttactaggatgactttttttttatacagcagaataaattatgacatttctattgaaaaaaaaaaaaaaaaaaaaaaaaacccacgcgtccgcggacgcgtgggtcgac

SEQ ID NO: 20 - DNA, 863 bp, Rattus norvegicus
gtgacatgctgtctagtccggtttcatctttttttttaatgttgtttatttttggatgtacaaaagaaaaattggggggagggggtgatctctgtagatactcttgtactttgaagttaccggaaatggaacgggtcttaaagcagaaagtaacttttccaaggaacagatgcttgcgaaggcccccttccttgtcttattctccagagacaactgaaatttagcttctttgttgcagcaaagctctttgcccaggtgaacactgaccaccgcgggttttctatgtcagaaagaagaagaaaaacaaaaacatgctcgagctttttctaacctccccttgggggtctgttgtgcgaacccctctttcttcaatatcgtgtcactttattctctttaatggactgtaacaaacaacaacaacaatgtaatcacgagagtgccaaatatcttgaaacgccaaaaggcattttggtttccttttctcccctgtgctctgagtcttcgtactggaacgcttggagtgtcttttctgttatttataggggttctcttaaggctctcgccagctgcctgttttgcatggtatttgcaaaaaaaaaaatgcctcttgcgtgaggaatcttttactttttttttttttatttgtttgcaactttggacctcaagaggtccccaccccagtcccagttccttcttttcttaattctttattctgtatgctgcaccttgaaccagcacacagggctatttctccaatgtacaataaagaacttcctgtgtctccttaaaaaaaaaaaaaaaaaaaaaaaaaaaaaaaaaaaaaaaaaaaaaaaaaaaaaaaaaaaaaaaaaaagggcggccgc

SEQ ID NO: 21 - DNA, 2485 bp, Rattus norvegicus
cccacgcgtccgggggaactcacccagcatatggggagccctagaattcttcccttagcttgactggggaaaaggaagggaaggaggaaggaaagaggaaaggggcagaaaaggagagagggagggaactacagaaatcagttatcaaccctggcaaaaacaaaagtgtgctaaaagtttaagtgtaatatgaaaagatgacagagaatagacattgagttgctgacaggaaggaccactaacgaagtaaggcttcctggtggtagaaagtagatgccaccctctctaaatttccaaaacatgacttgaggcaagtctatcacataaggcaagttgttaatgagtagttgcagaaatgaacaagagtatggtgatgtatgcccgtacagctaaagcccaatacttgagaggctcaggcaggagaaggatcaagggcaacacagtgagactctgcctctaaacaaatgcactgaaacaccacattatagaagcaaaacaggagactggaatttggggaatgttgatttttcaaccatagcacctattagacaatcagagatcctgcctacgaggacgcctcacaggactcaaggatctatacaagcttccgaagcctttccccaccaccctactatacatctctgttccattccctctcctgtgtgctaaaagatgctattcgtgtccttgtgatgctgaatgactgtgtacacatgcaggatgtgtgtctacaatgtcaaagttagttaaaaacaataatgaagtaaacacattccctcaaaccaaaaaaacaaaaacaaaaacaaaaaaaaccaaatagtaccttataaatgactatagaataaacaaaattttaaaacccacgaaaacttagttgcaggcaaggcaaatcaacaaacatccagatgtactaggaatgtaccaagtagttatgcccttcagaatataagataaagcaaagatatttttaaatgtcgagttttccttgttcatggggacccatttttaatttgtatctacaagattagatgtacaaagcagctctcttccaaagctagaaccggcataggtgtccattagcaagtagtagctatagaatactgctccattcatgaggaaaatctactcctaattaaaatagggagtatgtgtctctcacaaacaccctttgggggtaaaagaaaactttgcacgaaatatgacacactgcgtgatgtgattgtgtggggctctgaaacacacaaaagtagcctgtgctttaaaggagcattatagtcattgtctttgggagacagaagcctgattccatgcaaaagggctagaggtcactttctgagtcactactgagtgtgctagatggtcactgaggtacacagcttctccagaattcaggcaaacgtatgctcacaccacatgtactttgccttatgtagtggttctcaacctgtgggtcatgaatcatgttatgattcaaaataatagagaagtatgaagtagtaatgacataatgtcatggtcgggggtgacggcatgaagaactgtaacaaagggtcactgcatcaggaaagtggagagccactgccttaaagaaaccttagaatatatttctgaagtgttcaggaatgagggacattgaagtctgacacttactttaaaatatcaggagacactgataaaaattaagagatgagcagatgtgtgacaaagcaaacatgataaaatgtaggtggtatgttaatggtttgtatcctataatggtctcattttctgcgatagttcaaattttattttaaagtgtaaaaaaaataaactctgacagagctgacacaaggctgacacaaggctgacacaaggctgtctcttcgccatggtaaccatttcctttactgtgtatgagcttttgaatttaatatgatcctacttattaattcttgaaattactccctctgctgctggagttctttgcaaaaatgttcttgcctttgcctttgccctgccagtatcttaagagtatttttgtagatttttatcctgcagtttcaattttccaagttctatacaaggttttgctccattttgacttttgtgtgtgcaggatgaggaacgggaatctagtttcattgttttacgtgtgggtattcagtttttccagaacctgttgttgaagagggtgtctctttcaatgcatgttttgttacttttgtcaaaagttaggtggctgtcactgtgtgggcttatttctaagtcatccactaagtattccatcgctctccatgcttgtctttgtgccagtaccacatagtttctgttattatcacttcatgttataatttaatatcagatatttcatcagcgtcttcctgcttagaatcattttggtattcagggtcctttgcacttctatatgaatacctaggacctataaagtaactcaaaaatgaaacacccaaaaaaaaaaaaaaaaaaaaaaaaaaagggcggccgc

SEQ ID NO: 22 - DNA, 3381 bp, Rattus norvegicus
gtcgacccacgcgtccgcggacgcgtgggccggtttgagaggtgactgtgagctgggctcagtgctgccaccggtcacctaagggagcgctggcgaggcgcagactctcggcttagtcggccgcggcccaggctcccggcgcggcgcggaacggagtggcagaaatcttaaataattccatcagaatgacaccttctcaggtcacctttgaaataagaggaactcttttaccaggagaggtctttgcaatgtgtggaaactgtgatgccttgggaaactggagtcctcaaaatgctgtgcctcttactgagagtgagacaggcgaaagtgtatggaaagcagtgattgttcttagtagaggaatgtccgtgaagtaccgctacttcagaggctgctttttagaaccaaagactatcggtggtccatgtcaagtcatagttcacaagtgggagactcatctacaaccacgatcaataacccctttagaaaacgaaatcattattgacgatggacaatttggaatccacaatggtgttgaaacactggattctggatggcttacctgtcagactgaaataagactgcgtctgcatttttctgagaaacctcctgtttcaattaccaagaaaaagttcaaaaaatctagatttagggtaaagcttacactagagggtctggaggaagatgatgacgacgatgataaggcatctcccactgttcttcacaagatgtccaatagcctggagatatccttaataagtgacaatgagttcaagtgcaggcactcacagccagaatgtgggtatggcttacagcctgaccgctggacagagtacagcatacagacaatggagccggacaaccttgaactcatctttgacttttttgaggaagatctcagtgagcatgtagtccagggtgatgttcttcctggacatgtgggcacagcatgcctcctgtcatctaccattgctgagagtgagagaagcgctggaatccttactcttcccatcatgagcagaagttccagaaaaactataggcaaagtcagagttgattttatcatcatcaagccattaccaggatatagttgttctatgcagtcttcattctccaagtattggaaaccaagaataccactggatgttggacatcgtggtgcagggaactcaacaacaactgccaagctggctaaagtacaggaaaatactattgcttctttaagaaatgctgccagccatggtgcagcatttgtggaatttgatgtccacctttcaaaggacttagtgcctgtagtgtatcatgatctcacctgctgtttaactatgaaaaggaaatatgaagctgatccagttgaattgtttgaaatcccagtaaaggaattaacattcgaccaactccagttattgaagctttctcatgtgactgcactaaaaaccaaagaccagaaacaatgtatggctgaggaggaaaattccttttctgaaaaccaaccatttccttctcttaagatggttttagagtcattgccagaaaatgtaggatttaatatagaaataaaatggatttgccaacacagggatggagtatgggacggcaacttatcgacatattttgatatgaatgcatttttggatataattttaaaaactgttttagaaaattccgggaagaggagaatagtattttcttcatttgatgcagacatctgtacaatggttcggcagaaacaaaacaaatatcccatattatttttgacccaaggaaagtctgacatttaccctgaactcatggacctcagatctcggacaacacccattgcaatgagctttgcacagtttgaaaatattttggggataaatgcccatactgaagatctccttagaaacccatcctatgtccaagaggcaaaagataagggattggtcatattctgctggggtgatgataccaatgatcctgaaaacagaaggaaactgaaggaatttggagtaaatggtctaatatatgataggtatttgttttttgtaaaaaatctccatggaattgttcaaacagtgtagttttatctattttaactattttaaaattagatagtttagcctaaagttttatcttgacactgtgacctttcccaggtgttgagatatgtcaaaagccacttaagaagccctaacccaaatgtatttgccttgaagtgagggtacttgcctgtctcactcctgtctgtcaaaactttttctgcagttgtcttagttacattctattgctgtgaagaagtactgtgatcaaggtgaattgtttgcagttttggagggtgagcccatggctgtcatggtggggagtgtggcagaaggcaggcatggcactggagtagttagtagctgtcagcttacttctgatccacaagcaggagtcagagacacaggcagaaacagattgtccctggtgtggacttttgtaacctcaaagtttactgcctcataacaaacacacgaggccatacatacctcctaatctttcccaaatagtccaactggggaccatacttcctcattcaagggtctacagtgatctcttcctgtgtgggggtccttatcttcactcatatccaactcaggacacttccctgactttaaaacttttacgtcccttctcttgatttcagcctaaatggccactctgcttattttgctttctcaggccgaatccttgaagtcttccatggcagtgttcttgaacctcttcttcattcctattttcccctgggatcttaactcacacaacattcctcaatgggctctttctttctgcttcagagagtgactcccgtacactgaaatgtcacttgcacaaggactacactatttaaaatggtagcttctgttcatagggtgcttcttaactttgcttgagataatttatcacggacaaacacagatatatgtaacattgtattgtgtgtcttttccttcctggcctccccgtttcctaatgcatacacagagtgtgggctgcacaagaaccaggacactttcctttttttgtttactatcgtcaaaggctaagacaataatatgtagactgtgggatcagatatttgttgactggatacagcttcctaggaattggtatgtaagatgtaagtttaatagctgctgatgttcagaaagttgctttagtgtaaagaagctttaggttgtaaaaaaacgactcgatggaggaagtacaaagttttgaccagaccttgagaaaaaaaaccaaaataagctttccttagatttaattcctactactctttatcctactgagttgtaccattctttttaataaagactttactcccaaaaaaaaaaaaaaaaaaaaaaaaagggcggccgc

SEQ ID NO: 23 - DNA, 1596 bp, Rattus norvegicus
gcggccgcccttttttttttttttttttttttttttttttttttttttttttttttttttttttttttttttttttactaaaaacctttgaccagttttatttaacaagtgttgtacaaagagatttctgtaccgaaatgtttaccacaggcaccatactagaaacactagaaacatttagaaatctttgaatggagaaagcatacatgtttagtcagtaaacatgctgcaggtgtggggaaacacaccatgttaagtgaaaaggtgtgacaaaaccatctgatacaatttaacatatatatgtatgtataaaaaacatctatgtccatgcatctcaccagccagccagccccatcaccactggtggatcaggttgctgctttccctattgctactgctatctagaatctgaagttgtctaaaattaaagttgcttacaaacaggtattagttgtttcgttcatgcccaggatgagcttcctgacccccacgatcctgctgctggcgctggtcgccgccacccaggccgagcccctgcacttcaaggactgcggttctaaggtgggagttataaaggaagtgaatgtgagcccatgccctacccagccctgtcagctacacaaaggccagtcctacagtgtcaacgtcacctttactagcggcactcagtcccagaacagcacggccttggtccacggcatcttggcaggggtcccagtctacttccctattcctgagcctgacggttgtaaatgtggaatcaactgccccatccagaaagacaaggtctacagctacctgaataagctgccggtgaagagcgaatatccctctctaaaactggtggtggaatggaaacttcaagatgacaaaaaggataacctcttctgctgggagatcccagtagagatcaaaggctaggctgcttggtgccctgtgtctgtgcagggtgagaggccatgggcggagggaggggaaggaagagaaatcagacctgaaattgagtcggtgccataagacgaacagaacttcaagaatgctgttttatgcctttcagcctccaaaaacatacctgcagccctactactcttgagagccagagccatggccccctgagatagcctttgtggaggcttcgggagggaaaggggagactggagagattagattagtgtccatggctgtttgctgttggattacgtcggcaggtccaggcaagatgaggcagggatgcttgaggatgtcagataacctgtcaatccactgtgaaggatggcttcccagaatcttctggctggccgggagtattacctcttctgtatctaagtgcctcctgagtcccaagcaccctgcttatcgatccgatgagtctccatggtaccctctgcccaacgcttcaacagcagtgactaactctccatggtccagagacggcctgagggaaggtctgcgcagaaacttagctctgactggctgctgctttgcggttagctcttgttctttggtagttttcattaaagccaatacttggttgcaaaaaaaaaaaaaaaaaaaaaaaaaaaaaaaacccacgcgtccgcggacgcgtgggtcgac

SEQ ID NO: 24 - DNA, 3934 bp, Rattus norvegicus
cggacgcgtgggcggacgcgtgggctcagaaagccttagacacgcagcgtgtggcagaactaacctggcgtgtgagggttaattctgctcagtgcctccacccaagggacatggcccttccctgagggaatatacacggtagtgggtggtgtctacaggatgctcacgaggtgggactgtccccacagctccactcgggtgcctatgtgtcttgtgtgctggcatcgggagtgtgtgagagtcgaatttctcaaataggctcaacaccttccctgggcttctttttgtaaattgttctaaatttctgtgtagatcaaggaagccttgatgatttccggtgcatatattaacagctatataattaaaccatgttacataaggtgcctcatgtgggctgacaggctgcgtgaccaggccagaagagctcaaagggctccctggctcttaacaccactctacccagactgctttctgcttgtctgcccttttttttttttttttgtctgttttgtttgttttttggtgttttttgttttttaaactcttaacagccagctgtgaggaaagggctctgatttgctagttcggtgtggcaattagcacctggctagggagaaccagtggttctctgtgtctttgggacgcacgtcttatttccagttggataaaaggagcctttgcctcttgtagtgtccccatgaggttgaagggcctcgtggagacaaaggtacccatgctttcagcagggaatgctcatgtcacatcctcaggtacaagtccaagccaacctgtacgtggtgagcacaggcgtgccattatgggacctaaacctggccagatggagcaagggcacgcgagacaaaaggcgtaagggaaaacagaggcaacagcgcccaccctgctggctctagcccttgatcccctctgctggctgaccttgggcaaatgactcaacttctctgatctttgacaatcacataaaataatgggtgtactcaaggttggccgtgaatacaagaaatcaggcagaagggcctctgctccaggcaggtgctcagcaaactactagaaagtgtcactggtgtgtccaccattaagtttcaaaaaagaagtcatctgaggcctggctggattcctgcattccagctcaggtatattgtgttttctagaacagagattctaggactttctaagaactcagtgctttgcaggactcaggcattagctctgcccgcaatccatgagggaaaagctgggtcaggcaaggcagatctccaggtaaggccagggcccggtcctggagaaggacttcacttcagaggttacttcatagctagacttcagtgacattgttgcaaggcagtccctcgaggggttaacacagctgcatcccctgagttacagctccagtgttcgtaaaggcttcacctcagcctgagtggctggccactgtgtggaaactactgggcttgttccgtactctgtggctgagctcgggagacattgcacactcattctcgggaatatgactgcctcctattctgctgaggagtgtgtcgtacgtcgccatctctggactcacaatctgaatgcaatctttagaagatgtatgtagaatctttaatacaagacgggagacagaagcccagaaggatccaaacgattaaaaagaacaacacagaaagaaaaggagtgaagtcccccaaggtgtgtctaggaggagtgtgcccgaggtctgcctccttggtttccttgccgctgctgtccctgtaggctgcgcgacctctcgagctgattgggcgcgcttcatttttaatttcaaacttagtgtctaaagagccatcaattcaggggttcaaaagccttgtcgtgcccgcattcacacactcccgtgtgttgctagtgtcttttggccacagaggcaacagtgtactggcagggtgctttccctgtgcctggggcagctcttacactcatcggcaccgaagccactttcttgaacaccctgtggacagtggtcccagtcccaacttacactgtcctcaatctcataactgagaaaataagaaacagcttgtttggagcaaaataacaaagctatagcgttctccctgcaaaggcaatgctgtgggcgccttagacggactcacgccctgtggctcagggtcaaggggctttgccttaaactacaaactccagtcagggctttctgaaggagggtctgagagatcgaccgactataattctgtgtcctgggataccacttccggcccgaacggcttgtgattggacaaccacagaaccagcgctgtggagggaaagtgtcatcctgcagccacgcaggctcagtgcctaactcctgtcttttccttttcccagggaggaagagacagccgctctggatctcccatggcaagacgctgagagcctccctgctcagccttcccgaatcctgccctcggcttcttaatataactgccttaaacgtttaattctacttgcaccaaatagctagttagagcagaccctctcttaatcccgtggggctgtgaacgcggcggggccaggcccacggcaccctgactggctaaaactgtttgtccctttttatttgaagattgagtttcctcggggtcttctctgccccgacttgctccccgtgtaccttggtcgactccggaggttcaggtgcacggacaccctttcaagttcacccctactccatcctcagactttcttttcacggcgaggcgcacccctccagcttccgtgggcactgcggatagacaggcacaccgccaaggagccagagagcatggcgcaggggactgtgtggtccaggcttcctttgttttctttcccctaaagagctttgtttttcctaacaggatcagacagtcttggagtggcttacacaacgggggcttgtggtatgtgagcacaggctgggcagctgtgagagtccagagtggggtggccctggggacgcttccaggccagcggttccctgcaccccaccagctgatttcgagcgtggcagagggaaggaaaggggcgagcgggctgggcaatggacccgacaggaaacggggacttaggggaacacgctggagatgccatgtgtggctgccgaaggtcaccatctctcctcagtggctccccagagcaggtgcttttaagaaccctgtttcctctcagagcccagggagagtccaaggacatggcgcatcaggaagtgggactgcaggagttctctggtggcctcgtgctgtccctctggccacttctcactttagggtggtcagcggcagctcgccatggcagtgcccattggtgcacactaacctcagtggaaaagtaaccattccctgcctcttagaaagaactcattcttagttttaggagggttcctgtcgctgaatcaagtcgctgccctggatgcagggctggcctgggcgaccctccagggatgaggagctcagaattccagtcttctaatgtccacggacacctccccatccctctaacgtactgactatgtcttttgatttagcatgtcttctatagaccttccaaagagacccacactggcactgtcaccccctaggagggaaggtgatggttgatgtagcccgacgcgcatcttgttaatccgttctaattccgaggagagtgtgggtttaagataacacctattaatgcattgccacaataatgtgggggtaagagaaacgcagggacgaaacttccagaaacaaaccctccagatcgttccacaggagtgttcgccctccggtgtgactgaacgaccgaccttgcccatggcttcatccagacagcacagctgcagtatggctggacagaagcacctactgttcttggatattgaaataaaataataaacttgcaaaaaaaaaaaaaaaagggcggccgc

SEQ ID NO: 25 - DNA, 1788 bp, Rattus norvegicus
cggacgcgtgggtcctggacaaggcaacaggtgaagggctgatccgggccaaggagcctgtggactgcgaggcccagaaggagcacacctttaccatccaggcttatgactgtggagaggggcccgatggtgccaataccaagaagtctcacaaggcgaccgtgcatgttcgggtcaacgatgtgaatgagtttgccccagtctttgtggagcgtctctaccgtgctgcagtgactgaggggaagctgtatgatcgcatcttacgtgtggaagccattgatggtgactgctcccctcagtacagccagatctgctactatgagatccttacacccaacacccctttcctcattgacaatgatggcaacattgagaacacagagaagttacagtacagtggtgagaagctctataagttcacagtgacagcatatgactgtgggaagaagcgagcagcagatgatgctgaggtggaaatccaggtgaagcccacctgcaaacccagctggcaaggctggaacaaaaggcatgaaggtgcacgtgaacccctcgcagtccctgctcaccttggagggggatgatgtggagaccttcaaccatgccctgcagcacgtggcttacatgaacactctgcgctttgccacgcccggcgtcaggcccctgcgcctcaccaccgctgtcaagtgctttagtgaagagtcctgtgtctccatccctgaagtggagggctatgtggtggttcttcagcccgatgccccccagatccttctgagtggcacagctcattttgcccgcccagctgtggactttgagggacccgagggagtccccttgttccctgatcttcagatcacctgctccatttctcaccaggtggaggccaaagcagatgagagttggcagggcacagtgacagacacacggatgtcagatgagattgtacacaacttggacggctgtgagatttctctggtgggggatgacctagaccctgaacgcgagagcctgctcttggacatggcttccctgcagcagcgaggcctggagctcaccaacacatctgcctacctcaccattgctggggtggagaccatcactgtgtatgaagagatcctgaggcaggttcattatcagcttcggcacggagcagccctgtatgccaggaaattccgtctctcctgttcggagatgaatggccgatactccagtaacgaattcattgtggaggtcaacgtcctgcacagcatgaaccgggtggcccatcccagccacgtgctcagttcacagcagttcctgcaccggggtcaccagcctcctcctgagatggctggacacagcctggccagctcccaccggaactccatggtccccagtgctgcgactctcatcattgtggtatgcgtgggctttctggtgcttatggtcatcctcggcctcgtgcggatccactcccttcatcgccgtgtctcaggaactggtggaccctcaggggcttccgctgacccgaaagaccctgacctcttctgggatgactctgctctcaccattatcgtgaatcccatggagtcctaccagaaccagcagactggtgtggcaggggttgctggtggccagcaagaggaagaggacagcagtgattccgaagcagctgactcccccagcagcgatgaaagacgcatcattgagagccccccacaccgctattgaggctccagccctgccaaaagagagagaggcctgccctggggagacaggcacccaggaaaaaaaaaaaaaaagggcggccgc

SEQ ID NO: 26 - DNA, 1403 bp, Rattus norvegicus
gtcgacccacgcgtccgagcggttcttgggggcgcagggggcgcgtcgccctctgcccccgccggcaccctggccatgacaggcaagtcggtgaaggacgtggatcggtaccaggcggtcctggccaacctgctgctggaggaagataacaagttctgtgctgactgccagtccaaagggccgagatgggcctcctggaacatcggcgtgtttatctgcattcggtgtgctggaatccacaggaatctgggggtgcatatatccagggtaaaatcagtgaacctcgaccagtggactcaagaacagattcagtgcatgcaagagatggggaatggaaaagcaaaccgactctatgaagcctaccttcctgagacctttcggcgacctcagatagacccagctgttgaaggatttattcgagataaatatgagaagaagaaatatatggaccgaagtctggacatcaatgtccttaggaaagagaaggatgataagtggaaacgaggaagtgagcctgctccagagaaaaagatggaacccgttgtctttgagaaagtaaaaatgccacagaaaaaagaagacgcacagctacctcggaaaagctccccgaaatccgcagcccctgtcatggacttgttgggccttgatgctcctgtggcctgctctattgcaaacagtaagaccagcaatgccctagaaaaggatctagatcttttggcctctgttccatccccttcttcagtttccagaaaggctgtaggttccatgccaactgccgggagtgctggttctgtccctgaaaacctgaacctatttccagagccggggagcaagtcagaagaaacaggcaagaaacagctctccaaggactccatcctgtcactgtatggatcccagacgcctcaaatgcctgcccaagcaatgttcatggctcctgctcagatggcatatcccacagcgtaccccagcttccctggggttacaccacctaacagcatcatggggagcatgatgcccccaccagtcggcatggtagctcacccaggagcctctggaatggtcacccccatggccatgcccgcaggctatatggggggcatgcaggcttccatggcgggcatgccgagcgggatgatgaccactcagcaggccggctacatggcgagcatggcagccatgccccagactgtgtacggcgttcagccagctcagcaactgcagtggaacctcactcagatgacccagcagatggctgggatgaacttctacggagccaacggcatgatgagctatggacagtcaatgggcggtggaaatggccaggcagccaatcagactctcagtcctcagatgtggaaataaaagcaaagcacctgtaaaaaaaaaaaaaaaagggcggccgc

SEQ ID NO: 27 - DNA, 1298 bp, Rattus norvegicus
atcattttgaccagcaggtctgcagacccctcccccattgctgaaagtcctcccgtgcttggtgtgtgggaccacaggctccgccctccctgcccatcacgcttgcagttttgcttaggagctggcccttcctcccagtgcaggggccccacagcacctcagaccccaagtgtgtctggagtcccctgtcagccagggagaggacaccagcacctgggacctccagagaagccgcagtgagcggacttgtcgacagagggtaaaaaattactcccacgcagtcatcatttttcttcatttttaaaagtttttatttttattttccaatatagtgcatgtataaagtgggagagcggggagggggggttaatatgtagatgaccaactgactttttaatattttgtaaataaattgggattctttgtgtcctttgtgctagtgtagtccaggacaggaatgtgaagtcagaacatggggccaggaagagctcttcctcccttttccctcccaagaaaccaggttggaaaggtccaagtcacagtggcccatgctggggtttctgtacagccatgtggccagggccataggttttgagtgctgcctgggggagccagacccacgcgctcccaccacattagaggctgggaacagccaggatggtgccaaagcccctggccttccttgtacacgcctgtgaccagcctcgtggcttctgctaattagcgtgtctccctgttggtgaaaacctgtagctgggaactgatggcaaagatggacaacagttctgagcagtctgcactacagcacccaagaggagaacctgaggcccgaaaacaaactgctacaatgtttcaaaacgagctgcgctctcctccccagagaccccacgggatgcccccgggatgcccccttgctgtcgggttttggctaagacctaagacccagcagaggagagccagccggcttggggggtgggggtgggggaggagacagcagctaaaacccacacagcacggcttgtcattcacagtcacagtttagactcctccagctggggaatccggtcctcgctgctagtcctaaggatgttgacgctgtgctgcctgtggccaccctcccgtgtcctgttccctgtagtcgctttatagatggaaacaggctatgaagagggacactgtcgtgtgttggtagccgcaggctccccttaagatgtgtatattgaccccaggtcaggaagtgtatgcgttataataaagttctggttctaactccaaaaaaaaaaaaaaagggcggccgc

SEQ ID NO: 28 - DNA, 4015 bp, Rattus norvegicus
gcggccgcagcgccgcctgctcagcgcccgggtcagtaggagccagtccttcgcaggcgtcctcggcagccaagagcgcgggcccaggaacttcacggtcttcagcccaccagggcctcaacggaagcctttagtgctctcccgagtgtcaaggatgttttctgtggcccacccagcccccaaggtgccacagcctgagaggctggacctggtatatgctgctcttaagcggggactgacggcctacttggaagtgcatcagcaagagcaggagaagctccagcgccagataaaggagtccaagaggaattcccgcctaggattcctgtatgacttggataagcaagtcaagtccattgaacgttttcttcgacgactggaattccatgccagtaagattgatgagctgtatgaagcatactgtgtccagcggcgtctcagggatggtgcctacaacatggttcgtgcttatagcactgggtccccagggagtcgagaggcccgagacagcctggctgaggccacccgaggccatcgagagtatacagagagcatgtgtcttctggagaatgagctggaggcacagctgggcgagtttcatctccggatgaaagggttggctgggttcgccaggctgtgtgtgggcgatcaatatgagatctgcatgaaatacggccgccagcgctggaagttacgaggccgaatagagagtagtggaaagcaagtgtgggacagtgaagagactgtctttcttcctctgctcacagaattcctgtctatcaaggtgacagaattaaaaggactggctaaccatgtagtcgtgggcagtgtctcctgtgagaccaaggacctgtttgccgccctgccccaggttgtggctgtggatatcaatgatctcggcactatcaagctcagcctagaagtcatatggagtccctttgacaaggatgaccagccttcagctgcttctacagtcaacaaagcttccacagtcaccaagcggttttccacatatagccagagtccaccagatacaccctcacttcgggagcaggctttttataacatgttacggcggcaggaggagttagagaatgggacagcatggtccctgtcatccgaatcttctgatgattcatccagcccgcagctctcaggcactgctcgatactcatcaactcccaagcctctggtgcaacagcctgagcccctgcctgtccaagttaccttccgaaggccagagagcctctcctctggttccatggatgaagagccacctctgaccccagccctggtcaatgggcatgccccttacagtcggactctcagccacatcagtgaagccagcgtggatgctgccttgactgaggccatggaagctgtggacttaaaatgcccagccccagggcctagcccacttgtatatccagagtccacccatgtggagcatgtcagtagtgttcctcctgttgcagacaatggccgttctgccacaagtcctgccctaagtacagctggccctgcccccacatttatagaccctgcctcatctacacagctagacttagttcacaaagccacagactctggctcttctgagttgccaagcatcacacatactatggcaagctctacatatagtgctgtgagcccgatcaacagtgttccaggcctaacttccaccactgtaggttctacccacaaacccatgccctctcccctcacctctacaggctctatccccagtgtcacagactcaatccagactaccacaagcccaactcacaccaccccaagccctacccacactactgtaagccctacacatagcactccaagtcccacccataccactgtaagtcccagcaatgctgctctaagccccagcaatgctactccaagcctcagccacagtaccactagtcctactcaaaaagccacgatgtcaactcataccactagtgctgtgggcccagtccagaccactacaagtcccatttctacaactgtaagcccctccccttctgtagacactgctataatctccagttcctctgcagtaccctctgtcccaggccctgaagcacggccttgtagtcacccaacctctactccctacactaaagcagaccccacagcagcctgcacctcttctccgagtcttgcttcctctggtccaaaacccctcacaagccctgccccagactcgctagaacaaatccttaagagcccaagttcctctccgtcatccatagtccctgaaccccaacgttcagaacttagcctggccttggttgctcaagccccagtccctgaagccactggaggagctggggacagaaggctcgaagaggctctcaggaccctaatggctgccctggatgattatcgaggtcagttccctgagctccagggcctggagcaggaggtgactcggctggagagtctgctcatgcagaggcaaggcctgactcgaagccgggcctccagtcttagcatcaccgtggagcatgccctggagagcttcagcttcctcaatgatgatgaagatgaagacaatgacagtcctggggacaggcccacaagcagcccagaggttgtggctgaggaaagactagactcatcaaatgcccagtgtctaagcacagggtgttcagccctggatgctaccttggtccagcacctgtaccactgcagctgcctcctgctgaaactgggcacatttgggcccctgcgctgccaggaggcatgggccctggaacggctgctgagggaagctcgagtgctccaggaagtgtgtgagcacagcaagctgtggggaaatgctgtcacatctgcccaggaagtggtacagttctctgcctctcggcccggtttcctgaccttttgggaccagtgtacagagggactcaaccccttcctctgccctgtggagcaggtgctcctcactttctgcagccagtatggtgcccgtctttccctgcgccagccaggcttagccgaggctgtgtgtgtgaagttcctggaagatgctctggggcagaagctgcccaggaggccccactcaggccccggggagcagctcaccatcttccaattctggagttatgtagaagtcttggacagcccctccatggaagcctatgtgacagagaccgcagaggaggtgttactggtacagaacctgaactccgatgaccaggcagttgtgctgaaggctctaaggttagccccagaggggcgcctgaggaaggatgggcttcgggctcttagctccctgctggtccacggcaatagcaaagtcatggctgctgtcagcacccagcttcggagcctgtcacttggtcctgtcttccgggaaagggctcttctgtgcttcctggaccagcttgaggatgaggatgtgcagactcgagtggccgggtgcctggctttgggctgtatcaaggctcctgagggcattgagcccctggtgtacttgtgtcaaacagacacagaagctgtgagggaagctgcccggcagagcctccagcagtgtggggaagaaggacagtctgctcatcgccagctagaggagtccctggatgccctgccctgcctctttgggcccagcagcatggccagcacagcattctgaactctgattgccagctcccagtgctccttccctcattttcagggctcactaggcactggcagggagggtgagggctggttccagtcacctctccccacaaattcctatcaatgaaaatctaatatattcttctgttatcactggggttggtagaatgcctgaaatgaagtgcctcccagccggttctgcatagccacaaacagtgtcaggggcctgaccgtttggtcagcttgctctgcctcaccacatcccttggttttgtattttatttacagagttttacagataataaaaaagcaaaatgtgaaaaaaaaaaaaaaaaaaaaaaaaaaaaaagttctagatcgcgatctagaactagcggacgcgtgggtcgac

SEQ ID NO: 29 - DNA, 1837 bp, Rattus norvegicus
gtcgacccacgcgtccggaaaaacatttccttctcaagggcagcattccagcacctgcttgcatctctcagccatctttccttgatcctgtctgggaagtgtatctcagtgactccacaatttctctctttgtccttttgttttaaaagtaacactttctataaaatagctaaatgtccttgaaggggtttaatcgtgcaaaaagtaagcaatataatatataatgtacgcaacctttaatgacagtatctattttcttaggggtattattcagataccttcccagatctggatttaactttggtaattgttatctatatagaaaatcaacaatatcaaataaatatttcagtgttctggcataaagtcctcgtgagtaagaactaatgattttggaatttgctttgtgtctattgttatgtccctcatttcatttctgatttttaattcatgtattgtgtctcttgtgcattgttagtttgaggaaggggttgtctctcttgtggatcttctccagaagaagaagaagaagaagaagaagaagaagaagaagaaaaagaagaagaagaagaagaagaagaagaagaagaagaagaagtagaaaagacagctcttgctttccttgactttgtatttttctctttgtttctgaaggcttgatttcaaccctgagttcatttcctttcatcagatcctaccagactctctatgggatgaggctttgcctcactttgggcaggctggatgatgacattcagttgacaatgtggacatctcaagacagtcactggacaatggctgacacacttgtctgaagactggaaatgtttctctatcttggagaggaaagaaggaccctccaactacactgaactctaaggaaggttattcagatctcaagactggttaaccccccaaaactgaccctgctaccagttgactgtgattataaagaagccaacagccaacagctgcacagcagagagatgggggagatgctgggagagaaacatcatcccctgaaactgccctaagcgagaagcaggctaagaaggaaaaagagaggctgatagaagagctgcagctcattaccgaggagagaaatgacctgagagatcgcctgaggtttctgacagagagatccatgaacaacaggccacacttcaggccaaatccatattatgaagacctggagagaatggaggaggcagtcatgtcaattctgcacaacttagagatggagaacactgagatccatgagaacaaccataagctgaagaaggagatgaccttctctagaaacctgctcagccagctcctgatggagaacacttttaggaagaagttggtcccactgaagcaggagagcaaggaggtacatcttgattgggtaccgatccagaaatatttggttgaattcaacaagatcgataaagaccagcaacctccagaccccgcatcatctggtctcaaaaagtacaagagagctgaaattggacacacactagtaagagagcttcctgaagaataagttgctttctcaggagtccctgatgaccaacatcctgaatgaaaacaccacttgagagacaacttgggggactgcctttcattatgtgtgctagaggagaaatagcaatacatctgtgcttctaaatattcctaaatgttcttgttactatttttcacttttcaaatactttaaaacatttatatttattcattttcatcctctgtctttgtaaaacgtatttcttatgaataaaaattgaattctatctcctgaaaaaaaaaaaaaaaaaaaaaaaaaaaaaaaaaaaaaaaaaaaaaaaaaaaa

SEQ ID NO: 30 - DNA, 1042 bp, Rattus norvegicus
gtcgacccacgcgtccggcggttacaagctaagacatgttttcccatcttcgcaagcgttttgggagggggaacgtcgattctggagagactagagtgaaggagtctggcctttcgtctcaaagtaatgatggagaaagacagcacttctggggaatgtggaacgttgggagagaaacatcatcccctggcactgacctaagcaagaatcaggccatgaaggaaaaggagaggctgattaaagagctgcagctcattaccgaggagagaaatgacctgagagatcgcctgaagtttctaacagagagatccatgaagaacaggccacacttcaggccaaatccatattatgaagacctggagagaatggaggaggcggtcatatcaattctgcacaacttagagatggagaacactgaggtccatgagaacaaccataagctgaagaaggagatgaccttctctagaaacctgctcagccagcccctgatggagaacacatgtaggaagaagttgttccccctgaagcaggagagcaaggaggtacatcttgattgtgcactgaaccagaaatatttggttgacttcaaccagaaagataaagaccatcaacggccagaaccagcattatcaggtctcagaaagtgcaagagagctggaattggacacaccccagtaagagagcttcctgaagaataagttgctttctcaggagtccctgatgacaaatatcctgaatgaaaacagcacttgagagacaacttgggggaccgcctttcattatgtgtgctagaggagaaacagcaatatgtctgtgcttctaaatgttcgttaagaatatgcttttagaaatatttttgttatgattttaattgaagttttctttttgttgtttcatatttatatgttcttgttactatttttactttcaaatatttttaaatatttttattcattttaatcctgttttgttgtaaaaatgtatttgttatgaataaaaattgaattctaaaaaaaaaaaaaaaaaaaaaaagggcggccgc

SEQ ID NO: 31 - DNA, 2393 bp, Rattus norvegicus
gtcgacccacgcgtccggtgcaaacattcaaaaatagtaaaacagattgatctctccctttttctaatagaaatatgaagctgatccagttgaattgtttgaaatcccagtaaaggaattaacattcgaccaactccagttattgaagctttctcatgtgactgcactaaaaaccaaagaccagaaacaatgtatggctgaggaggaaaattccttttctgaaaaccaaccatttccttctcttaagatggttttagagtcattgccagaaaatgtaggatttaatatagaaataaaatggatttgccaacacagggatggagtatgggacggcaacttatcgacatattttgatatgaatgcatttttggatataattttaaaaactgttttagaaaattccgggaagaggagaatagtattttcttcatttgatgcagacatctgtacaatggttcggcagaaacaaaacaaatatcccatattatttttgacccaaggaaagtctgacatttaccctgaactcatggacctcagatctcggacaacacccattgcaatgagctttgcacagtttgaaaatattttggggataaatgcccatactgaagatctccttagaaacccatcctatgtccaagaggcaaaagataagggattggtcatattctgctggggtgatgataccaatgatcctgaaaacagaaggaaactgaaggaatttggagtaaatggtctaatatatgataggatatacgattggatgcctgaacaaccaaatatattccaagtggagcaactggagcgcctgaagcgagaattgccagagcttaagaactgtttgtgtcccactgttagccacttcattcctccttctttctgtatggagtctaaaatccatgtggatgctaacggcattgataatgtggagaacgcttagttcctagtgcacagaggacattcagaggctctcccctgcgctgaggttccgtctccaccactgaacaccggtcgcctcttaggtttctcagtccaatgaagcaataatgaagtattttactatcattacagttcccgcaagaatatcaagtacactatttatcacttgtccaggtataattaccaatcagtctctgtacaaatgttaaacactttaaatgagagatctaagcctataatggtgaatcttcattaaaagcataatacttggaaattagctatataaatatctcatagttcaggcttttcatttgattaggtcttaaatcttcagtgcttgagaaaaatgactgcataattatacctgaccatggaaataataagtacctcaagtgcatgcatttgcactggtggctccagctgcacaagtctgtgtcatccatgtacataggtgtctttacatgggttgatagaaacatgcactaggctcctttagtataaacctcagactgctgccatttcccacctgacccaaaccagcctgcagatgaacctcaaaacttgtttcatagactgttcaaagattttaaaagttccagaatgctgcagggtaacttaatgtataaagtatttgtaagaggtatatattgcatatatagtcgtgtagatcagaatgtgtaaatttgactccgtgcatgctttaggtttgttttaagcatgaggttgtacatgtttactgtccttgcatctggtgctaggtgagtgagatgttaaggactgaaaatattttgtcgcctaaaaagagtatgcctatttagtgtctcactacctagtaagcaattgccgtgtgtgctccacagtagttaacccccatgcagaaaaataaatgtcctgaattctcaaattgctattctttattgtttaatatatatcatgtatgtaatttatttaggaatgtagaaattactgtatataaatgcatgcttttaggattatactaagatttcattagaagcagattgtattgataaaactgtaacttcagaatgaatgttaaataaaatgacagatttattttttcctcatcttaaaatgaaatttgaagaaggtattttgtagaattgttttataatcgtatctgtctgacaatagtcatttattcattttgtatggctggctcaccgcacgctctgtgttcctgctggggtagtttgttcatgtattgacattttgacagaagctaaaatcctaagacttgagatgacaagttgtaccttttatttttttaattttttgggacaacctgtgtagccttggctgtccaggaactcactgtgtaaaccagctggctttgaactcccagatcatctgcctctgcttcctaagtggtattaaagtcatgcgccaccaaaaaaaaaaaaaaaaaaaaaaaaaaaaaaaaaaagggcggccgc

SEQ ID NO: 32 - DNA, 3604 bp, Rattus norvegicus
gcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgtgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaaaaaaaaaaaaaaaaaaaaaaaaaaa

SEQ ID NO: 33 - DNA, 3583 bp, Homo sapiens
cggacgcgtgggcgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggttggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggtttcctagctcttgcctcagaccttaaagagagagggtctgatggggatgggcactggagacggagcatcccagcatttcacatctgagctggctttcctctgccccaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgtgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaaaaaaaaaaaaaaaaaaaaaaagggcggccgc

SEQ ID NO: 34 - DNA, 5145 bp, Homo sapiens
ggcggcgggagagctgctggctcgcccggatcccgggagctgcctggaggcgggcccggcccggggaaggtgagcggctgcgggacccagcccctcgccgggagcgggcaccatggtgctgtcggtgcctgtgatcgcgctgggcgccacgctgggcacagccaccagcatcctcgcgttgtgcggggtcacctgcctgtgtcggcacatgcaccccaagaaggggctgctgccgcgggaccaggaccccgacctggagaaggcgaagcccagcttgctcgggtctgcacaacagttcaatgttaaaaagtccacggaacctgttcagccccgtgccctcctcaagttcccagacatctatggacccaggccagctgtgacggctccagaggtcatcaactatgcagactattcactgaggtctacggaggagcccactgcacctgccagcccccaacccccgaatgacagtcgcctcaagaggcaggtcacagaggagctgttcatcctccctcagaatggtgtggtggaggatgtctgtgtcatggagacctggaacccagagaaggctgccagttggaaccaggcccccaaactccactactgcctggactatgactgtcagaaggcagaattgtttgtgactcgcctggaagctgtgaccagcaaccacgacggaggctgtgactgctacgtccaagggagtgtggccaataggaccggctctgtggaggctcagacagccctaaagaagcggcagctgcacaccacctgggaggagggcctggtgctccccctggcggaggaggagctccccacagccaccctgacgctgaccttgaggacctgcgaccgcttctcccgtcacagcgtggccggggagctccgcctgggcctggacgggacatctgtgcctctaggggctgcccagtggggcgagctgaagacttcagcgaaggagccatctgcaggagctggagaggtcctactatccatcagctacctcccggctgccaaccgcctcctggtggtgctgattaaagccaagaacctccactctaaccagtccaaggagctcctggggaaggatgtctctgtcaaggtgaccttgaagcaccaggctcggaagctgaagaagaagcagactaaacgagctaagcacaagatcaaccccgtgtggaacgagatgatcatgtttgagctgcctgacgacctgctgcaggcctccagtgtggagctggaagtgctgggccaggacgattcagggcagagctgtgcgcttggccactgcagcctgggcctgcacacctcgggctctgagcgcagccactgggaggagatgctcaaaaaccctcgccggcagattgccatgtggcaccagctgcacctgtaaccagctgcccagctgcctcccttcttggacagccctgacccgtcctctgcaacctcctttctgtgcccctttcctcattctgacacccagaagacagtgacagatgtgtttgcaaggctgggatggctctctcatcatactcttgtttcttagaaataagcaagacagagcaggaaatggaatatgcgggtcacactgaggaatgcattttgctcatctgtgttattgaaggaggtgcttattaaatacagttcctatgcctgttttataggtggggttaggccagatgcagagaaagctaaatgtgggaatcatggatgcaaagaagaatttggctttttgaaaaacaagcatttcaaaaatgatgaaggaagtgaaagtatcctggatcaactcctagagttagagattgcccaggtggaaagaaaccttagccagcgttcaatcaagctcaccatgcagggcagtcacccggcagttctcaaactttagcatgtgaagagtcaccagcagattcctgggctcgcctggagacattcctagtcggtattcctggtcgaagcccaggagccttcctttttaacaagctgatgtagagggtggagcactgtatgtggagaaattccttctacaatattccacacaggtttttggccacagtccttgatggagtcccaaaaccatggtgcagccagttccaatgctggacacctcaaccatcagggtgaaatctggggcctcagctttttaatttaattattttaattcttaatactttaatttgtgcatttcataagccccctgctcttggactgaattttgtgctttttattgaagaattttattgtttttatcttaaaatcagtttctattatccttggggagaccatccctaacaaagtacaggtgggatctcctgtgagtcattggctgggttctgattgctagatgtcacacccaccagcatcaccaaagtgactctgagatagaccggtcccttctcagcgttccagtcacttcaggaggaatttagttattgacttagtctatgacatctggctacatgtaggtagagaagaaagacaattttaaaaaggaaatcaggtcttttgcaactgtgcctccctctgtctgttttcacttgaatgggtaaataaccagcagctaggttttgaattcctaccttgttattctaaacagatgtccacattgttaattaaatctaaattatgagccttgctgagtggatacggtacttacacctgaaccaggattcctgggttctgttgttgacattgcccttcagcacctgtttggccagctgtataagataggactaatgactaggaagcctaccccaatgaatgatatactagatgaaatagtgttcaaaacctgtaggcactctctggctaaaaacaaactctgaggccaccagcagatcatctttaagctaagttactatttttcacctttttttttagacggagttttgctctttgttgcccaggctggagtgcagtggcacgatctcggctcactgcaacctccgcctcccaagttcaagcgattctcctgtctcagcctcctgggtagctgggattacaggtgcccaccaacatgcctggctaatttttgtacttttagtagagatggggtttcaccatgttggccaggctggtcttcaactccagatctcaggtgatctaccctcctcggcctcccaaagtactgggattacaggcctgagccaccgcgcccggcctatttttcactttaatttggcagctgagaatgcccaaaaagtgccagaagcatcgtggcatttccagaaccatggattctgcctttggacccctctctattaatattaaaactctgggccttcagatgtcaccctaatccactgccctaagacagaatttctggacaagatgggtaagggcttcattccttcaacaagtcaagtcatacttggcctctccctgagaatctgagcaggagccttataacctgtggtcattattttttctttctgtacagaaatagaaaagcattagaaataacttctaaccatcctctgaaaaaacagaaaaaatatcgaatccctctttcatgagaagtcttttggataattggaaaccttcatcactgaggttggccagcccctgccaagtgttgtgtaggcaaagcacttgttagtggcttcctatgaaatgttttagagatctcttcaccatactggtttcttctctttggttggtgtgggtaaaagaaaacaaaacatttcctataagctgaaagctgaccagcattctcttcttggtaacatctactactccaacctagaaaatttggattctagaccaaaaatcaggaaacatggctccttataaatctgtgcagctgccttatagtaccatcaaaggaatttcaggtgggctgggcggggccccgatcccagaattatcaactccacccatcatcatttggtcatgaagcatcctttcattcttcttcttcttttttttggggggggcggggcgggggagggatctcaaagttttagtcttccagaatccaaattaaaggttgcccctgatgggggccaggttccgccacagaacatcttagatgtcagccttgacctcacttagcagggattacagaaatgagatacattttgaaggagagttgtctgttatgttcactgtattctaagtgcctgggataaagctgtctcatgggtgctccatatatattcatatatatttgttgagtgaattaatgaattaagagtggctggcagagtaggcagaaaaagacactgcaaatggcataaaaattaaagtcctagctgagttctcaatggtaaaggcatcagatgtcttagcagtcaagctagaaattcatgacaatgagtattactatttgcctaatgacaactcattgctctccatgtaaatgtaatcaacagatgaagagaatataattgctctgcttttccactaaaactccatcttagtgaattttaaattatccagagatgtcaaactgccaaataaaaatatttcagtagtctttgcatcagcttaccttgtaccagaaacatttccaatttactatcaaattatagtaactgagcctgtgtgaagtatctcatcattttcgaaaggaacaccttgtgtgatgccagtgagcatttctaaaaagggtgtgaggtagaggtaaaaataaggtgagagaccatttcagaatgcactgttgctcaaaaaggtgatctggttctttcttcagagatttctacggggatagaaaatcgggagtctgccctcattaatctgtgactccacctcttgcatcaaatcaatatctatttgttgagcacttattgattaagaccttgcatatgtctgtccattttgatttgagatacaactttttgtgtgggttgaatgacaaatcactccaaacaaaactgggcacagagaatcagctaggagaccagttattcagggtccatttctcttggatgtaaaggagtcctgggtaaaatgtggctgtaacctaaaccaactagtccttgtgatttgtttctgccctctgtgtttcctgttgtcaaatgctaagtgtgtgttttgcagtcatgaactaaagcacaaaaagatgcatgagacattgtagtcatatgtctggtgtgacactttggagcaaaaaccttgcagtggtaaataaaaaatttccaacagggt

(19) United States
(12) Patent Application Publication
(10) Pub. No.: US 2011/0230392 A1
(43) Pub. Date: Sep. 22, 2011

(54) NOVEL NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, AND NARC 25 MOLECULES AND USES THEREFOR

(75) Inventors: Lillian Wei-Ming Chiang, Princeton, NJ (US); Andrew Wood, Newton, PA (US); Lorayne P. Jenkins, Highstown, NJ (US)

(73) Assignee: Millennium Pharmaceuticals, Inc., Cambridge, MA (US)

(21) Appl. No.: 13/071,809

(22) Filed: Mar. 25, 2011

Related U.S. Application Data
(63) Continuation of application No. 12/817,236, filed on Jun. 17, 2010, now abandoned.
Application No. 12/817,236 is a continuation of application No. 12/316,681, filed on Dec. 16, 2008, now Pat. No. 7,776,577.
Application No. 12/316,681 is a division of application No. 11/313,836, filed on Dec. 21, 2005, now Pat. No. 7,482,147.
Application No. 11/313,836 is a division of application No. 10/426,776, filed on Apr. 30, 2003, now Pat. No. 7,029,895.
Application No. 10/426,776 is a continuation-in-part of application No. 09/692,785, filed on Oct. 20, 2000, now abandoned.
(60) Provisional application No. 60/161,188, filed on Oct. 22, 1999.

(51) Int. Cl. A61K 38/02; C07H 21/00; C12N 5/10; C07K 2/00; C07K 16/00; C12P 21/00; G01N 33/48; G01N 33/68; G01N 33/53; A61K 31/7088; A61K 31/7105; A61P 25/28

(52) U.S. Cl. 514/1.1; 536/23.1; 435/325; 530/300; 530/389.1; 435/69.1; 436/94; 436/86; 435/7.1; 514/44 A; 514/44 R

(57) ABSTRACT

The invention provides isolated nucleic acids molecules and proteins, designated NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, and NARC 25, nucleic acid molecules and proteins. The invention also provides antisense nucleic acid molecules, recombinant expression vectors containing said nucleic acid molecules, host cells into which the expression vectors have been introduced, nonhuman transgenic animals in which a said genes have been introduced or disrupted, fusion proteins, antigenic peptides and antibodies to said proteins. Diagnostic and therapeutic methods utilizing compositions of the invention are also provided.

### RELATED APPLICATIONS

The present application is a continuing application of and claims priority under 35 U.S.C. §120 to U.S. patent application Ser. No. 12/817,236, filed Jun. 17, 2010 (pending, the “'236 application”). Some subject matter has been removed in the present application as compared with the parent '236 application; no new subject matter has been added. The '236 application is a continuation of and claims priority under 35 U.S.C. §120 to U.S. patent application Ser. No. 12/316,681, filed Dec. 16, 2008, now U.S. Pat. No. 7,776,577, which is a divisional of and claims priority under 35 U.S.C. §120 to U.S. patent application Ser. No. 11/313,836, filed Dec. 21, 2005, now U.S. Pat. No. 7,482,147, which is a divisional of and claims priority under 35 U.S.C. §120 to U.S. patent application Ser. No. 10/426,776, filed Apr. 30, 2003, now U.S. Pat. No. 7,029,895. U.S. patent application Ser. No. 10/426,776 is a continuation-in-part of and claims priority under 35 U.S.C. §120 to U.S. patent application Ser. No. 09/692,785, filed Oct. 20, 2000 (abandoned, the “'785 application”). The present specification contains only subject matter found in the '785 application and therefore is properly considered a continuation (rather than a continuation-in-part) of the '785 application. The '785 application claims priority under 35 U.S.C. §119 to U.S. Provisional Application Ser. No. 60/161,188, filed Oct. 22, 1999 (expired). The entire contents of each application listed in this priority chain is specifically incorporated herein by reference.

### SEQUENCE LISTING

The present specification makes reference to a Sequence Listing (submitted electronically as a .txt file named “Sequence Listing.txt” on Mar. 11, 2011). The .txt file was generated on Mar. 9, 2011 and is 93 kb in size. The entire contents of the Sequence Listing are herein incorporated by reference.

### NUCLEIC ACID MOLECULES DERIVED FROM RAT BRAIN AND PROGRAMMED CELL DEATH MODELS

### Background of the Invention

A great deal of effort has been expended by the modern scientific research community to identify and sequence genes, particularly human genes. The identification of genes and knowledge of their nucleic acid sequences pave the way for many scientific and commercial advancements, both in research applications and in diagnostic and therapeutic applications. For example, advances in gene identification and sequencing allow the production of the products encoded by these genes, such as by recombinant and synthetic means. Furthermore, identification of genes and the products they encode provide important information about the mechanism of disease and can provide new diagnostic tests and therapeutic treatments for the diagnosis and treatment of disease. Thus, identification and sequencing of genes provide valuable information and compositions for use in the biotechnology and pharmaceutical industries.

In multicellular organisms, homeostasis is maintained by balancing the rate of cell proliferation against the rate of cell death. Cell proliferation is influenced by numerous growth factors and the expression of proto-oncogenes, which typically encourage progression through the cell cycle. In contrast, numerous events, including the expression of tumor suppressor genes, can lead to an arrest of cellular proliferation.

In differentiated cells, a particular type of cell death called apoptosis occurs when an internal suicide program is activated. This program can be initiated by a variety of external signals as well as signals that are generated within the cell in response to, for example, genetic damage. Dying cells are eliminated by phagocytes, without an inflammatory response.

Programmed cell death (PCD) is a highly regulated process (Wilson (1998) Biochem. Cell. Biol. 76:573-582). The death signal is then transduced through various signaling pathways that converge on caspase-mediated degradative cascades resulting in the activation of late effectors of morphological and physiological aspects of apoptosis, including DNA fragmentation and cytoplasmic condensation. In addition, regulation of programmed cell death may be integrated with regulation of energy, redox- and ion homeostasis in the mitochondria (reviewed by Kroemer (1998) Cell Death and Differentiation 5:547), and/or cell-cycle control in the nucleus and cytoplasm (reviewed by Choisy-Rossi and Yonish-Rouach (1998) Cell Death and Differentiation 5:129-131; Dang (1999) Molecular and Cellular Biology 19:1-11; and Kasten and Giordano (1998) Cell Death and Differentiation 5:132-140). Many mammalian genes regulating apoptosis have been identified as homologs of genes originally identified genetically in Caenorhabditis elegans or Drosophila melanogaster, or as human oncogenes. Other programmed cell death genes have been found by domain homology to known motifs, such as death domains, that mediate protein-protein interactions within the programmed cell death pathway.

The mechanisms that mediate apoptosis include, but are not limited to, the activation of endogenous proteases, loss of mitochondrial function, and structural changes such as disruption of the cytoskeleton, cell shrinkage, membrane blebbing, and nuclear condensation due to degradation of DNA. The various signals that trigger apoptosis may bring about these events by converging on a common cell death pathway that is regulated by the expression of genes that are highly conserved. Caspases (cysteine proteases having specificity for aspartate at the substrate cleavage site) are central to the apoptotic program, are. These proteases are responsible for degradation of cellular proteins that lead to the morphological changes seen in cells undergoing apoptosis. One of the human caspases was previously known as the interleukin-1β (IL-1β) converting enzyme (ICE), a cysteine protease responsible for the processing of pro-IL-1β to the active cytokine Overexpression of ICE in Rat-1 fibroblasts induces apoptosis (Miura et al. (1993) Cell 75:653).

Many caspases and proteins that interact with caspases possess domains of about 60 amino acids called a caspase recruitment domain (CARD). Apoptotic proteins may bind to each other via their CARDs. Different subtypes of CARDs may confer binding specificity, regulating the activity of various caspases. (Hofmann et al. (1997) TIBS 22:155).

The functional significance of CARDs have been demonstrated in two recent publications. Duan et al. (1997) Nature 385:86 showed that deleting the CARD at the N-terminus of RAIDD, a newly identified protein involved in apoptosis, abolished the ability of RAIDD to bind to caspases. In addition, Li et al. (1997) Cell 91:479 showed that the N-terminal 97 amino acids of apoptotic protease activating factor-1 (Apaf-1) was sufficient to confer caspase-9-binding ability.

Thus, programmed cell death (apoptosis) is a normal physiological activity necessary to proper and differentiation in all vertebrates. Defects in apoptosis programs result in disorders including, but not limited to, neurodegenerative disorders, cancer, immunodeficiency, heart disease and autoimmune diseases (Thompson et al. (1995) Science 267:1456).

In vertebrate species, neuronal programmed cell death mechanisms have been associated with a variety of developmental roles, including the removal of neuronal precursors which fail to establish appropriate synaptic connections (Oppenheim et al. (1991) Annual Rev. Neuroscience 14:453-501), the quantiative matching of pre- and post-synaptic population sizes (Herrup et al. (1987) J. Neurosci. 7:829-836), and sculpting of neuronal circuits, both during development and in the adult (Bottjer et al. (1992) J. Neurobiol. 23:1172-1191).

Inappropriate apoptosis has been suggested to be involved in neuronal loss in various neurodegenerative diseases such as Alzheimer's disease (Loo et al. (1993) Proc. Natl. Acad. Sci. 90:7951-7955), Huntington's disease (Portera-Cailliau et al. (1995) J. Neurosc. 15:3775-3787), amyotrophic lateral sclerosis (Rabizadeh et al. (1995) Proc. Natl. Acad. Sci. 92:3024-3028), and spinal muscular atrophy (Roy et al. (1995) Cell 80:167-178).

In addition, improper expression of genes involved in apoptosis has been implicated in carcinogenesis. Thus, it has been shown that several “oncogenes” are in fact involved in apoptosis, such as in the Bcl family.

Accordingly, genes involved in apoptosis are important targets for therapeutic intervention. It is important, therefore, to identify novel genes involved in apoptosis or to discover whether known genes function in this process.

Nucleic acid probes have long been used to detect complementary nucleic acid sequences in a nucleic acid of interest (the “target” nucleic acid). In some assay formats, the nucleic acid is tethered, i.e., by covalent attachment, to a solid support. Arrays of nucleic acid sequences immobilized on solid supports have been used to detect specific nucleic acid sequences in a target nucleic acid. See, e.g., PCT patent publication Nos. WO 89/10977 and 89/11548. Others have proposed the use of large numbers of nucleic acid sequences to provide the complete nucleic acid sequence of a target nucleic with methods for using arrays of immobilized nucleic acid sequences for this purpose. See U.S. Pat. Nos. 5,202,231 and 5,002,867 and PCT patent publication No. WO 93/17126.

The development of specific microarray technology has provided methods for making very large arrays of nucleic acid sequences in very small physical arrays. See U.S. Pat. No. 5,143,854 and PCT patent publication Nos. WO 90/15070 and 92/10092, each of which is incorporated herein by reference. U.S. patent application No. 082,937, filed Jun. 25, 1993, describes methods for making arrays of sequences that can be used to provide the complete sequence of a target nucleic acid and to detect the presence of a nucleic acid containing a specific nucleotide sequence. Thus, microfabricated arrays of large numbers of nucleic acid sequences, called “DNA chips” offer great promise for a wide variety of applications.

### SUMMARY OF THE INVENTION

The present invention is based on the identification of novel nucleic acid molecules derived from rat brain and programmed cell death cDNA libraries.

Thus, in one aspect, the invention provides an isolated nucleic acid molecule that comprises a nucleotide sequence selected from the group consisting of the sequences shown in SEQ ID NOS:1-34 and the complements of the sequences shown in SEQ ID NOS:18-51.

The invention also provides an isolated fragment or portion of any of the sequences shown in SEQ ID NOS:1-34 and the complement of the sequences shown in SEQ ID NOS:1-34. In some embodiments, the fragment is useful as a probe or primer, and/or is at least 15, more preferably at least 18, even more preferably 20-25, 30, 50, 100, 200 or more nucleotides in length.

In another embodiment, the invention provides an isolated nucleic acid molecule that comprises a nucleotide sequence that is at least about 60% identical, about 65% identical, about 70% identical, about 80% identical, about 90% identical, about 95% identical, about 96% identical, about 97% identical, about 98% identical, or about 99% or more identical to a nucleotide sequence selected from the group consisting of the sequences shown in SEQ ID NOS:1-34 and the complements of the sequences shown in SEQ ID NOS:1-34.

In another embodiment, the invention provides an isolated nucleic acid molecule that hybridizes under highly stringent conditions to a nucleotide sequence selected from the group consisting of the sequences shown in SEQ ID NOS:1-34 and the complements of the sequences shown in SEQ ID NOS:1-34.

The invention further provides nucleic acid vectors comprising the nucleic acid molecules described above. In one embodiment, the nucleic acid molecules of the invention are operatively linked to at least one expression control element.

The invention further includes host cells, such as bacterial cells, fungal cells, plant cells, insect cells and mammalian cells, comprising the nucleic acid vectors described above.

In another aspect, the invention provides isolated gene products, proteins and polypeptides encoded by nucleic acid molecules of the invention.

The invention further provides antibodies, including monoclonal antibodies, or antigen-binding fragments thereof, which selectively bind to the isolated proteins and polypeptides of the invention.

The invention also provides methods for preparing proteins and polypeptides encoded by isolated nucleic acid molecules described herein by culturing a host cell containing a vector molecule of the invention.

Additionally, the invention provides a method for assaying for the presence of a nucleic acid sequence, protein or polypeptide of the present invention, in a biological sample, e.g., in a tissue sample, by contacting said sample with an agent (e.g., an antibody or a nucleic acid molecule) suitable for specific detection of the nucleic acid sequence, protein or polypeptide.

A general object of the invention is to provide a microarray of unique nucleic acid sequences useful for analyzing gene expression in various biological contexts including, but not limited to, development, differentiation, and pathological states, in vitro and in vivo.

More specific objects include, but are not limited to, use of the microarray to discover specific patterns of gene expression in those biological contexts.

More specific objects of the invention include the discovery of genes associated with development, differentiation, and pathological states, both in vitro and in vivo.

More specific objects of the invention include, but are not limited to, functional gene discovery, in other words, assigning a function to a previously uncharacterized gene sequence.

More specific objects of the invention include, but are not limited to, use of the microarray to obtain candidate target genes for diagnosis and treatment.

More specific objects of the invention include, but are not limited to, use of the microarray to discover compounds that are useful for diagnosis or treatment based on one or more sequences in the array.

Accordingly, the invention provides a unique microarray of nucleic acid sequences useful for analyzing gene expression in various biological contexts including, but not limited to, development, differentiation, and pathological states in vivo and in vitro.

The invention is also directed to one or more variants or fragments of one or more of the nucleic acid sequences that constitute the microarray.

The invention is also directed to the use of the microarray to discover specific patterns of gene expression in those biological contexts.

The invention also provides a method to discover genes associated with development, differentiation, and pathological states in vivo and in vitro.

The invention also provides a method for functional gene discovery, that is, a method to assign a function to an uncharacterized gene sequence.

The invention also provides the use of the microarray to obtain candidate-target genes for diagnosis and treatment.

The invention also provides use of the microarray to discover compounds that are useful for diagnosis or treatment based on one or more sequences in the microarray.

In a specific disclosed embodiment, the invention provides a microarray of genes associated with programmed cell death (PCD) (apoptosis). Specifically, genes whose expression is associated with programmed cell death in rat cerebellar granule neurons (CGN) were identified.

The invention also provides a kit comprising a nucleic acid probe which hybridizes to a nucleotide sequence of claim 1 and instructions for use, and a kit comprising an agent which binds to a polypeptide of claim 10 and instructions for use.

The inventors sequenced the 5′ ends of an extensive group of partial and full length cDNA clones and grouped these sequences into clusters based on nucleic acid sequence homology, assembled each cluster into a cDNA consensus sequence based on contiguous 5′ cDNA sequences, and placed a unique cDNA from each cluster into a microarray. The microarray was constructed with approximately 7296 cloned cDNA sequences. The microarray was then used for transcriptional profiling in various tissues and in two programmed cell death model systems. Expression data were analyzed with an expression pattern clustering algorithm. cDNAs with similar expression patterns were grouped together. Approximately 500 cDNAs were discovered to be regulated in programmed cell death models. These cDNAs are useful for diagnosis and treatment of programmed cell death-related conditions and for the discovery of compounds useful for treatment and diagnosis of programmed cell death related conditions. The cDNAs are further useful to discover other nucleic acid sequences whose expression is related to programmed cell death.

The invention is thus also directed to subarrays, in various biological groupings, such as a programmed cell death microarray.

The invention is thus also directed to one or more variants or fragments of one or more nucleic acid sequences in a subarray.

### DETAILED DESCRIPTION OF THE INVENTION

### I. Isolated Nucleic Acid Molecules

The invention encompasses the discovery and isolation of nucleic acid molecules that are expressed in rat brain and in programmed cell death in vitro models.

Accordingly, the invention provides isolated nucleic acid molecules comprising a nucleotide sequence and the complements thereof. In one embodiment, the isolated nucleic acid molecule has the formula: 5′(R1)n-(R2)-R3)m 3′

wherein, at the 5′ end of the molecule R1 is either hydrogen or any nucleotide residue when n=1, and is any nucleotide residue when n>1; at the 3′ end of the molecule R3 is either hydrogen, a metal or any nucleotide residue when m=1, and is any nucleotide residue when m>1; n and m are integers between about 1 and 5000; and R2 is a nucleic acid having a nucleotide sequence selected from the group consisting of the sequences disclosed herein and the complements of the sequences disclosed herein. The R2 nucleic acid is oriented so that its 5′ residue is bound to the 3′ molecule of R1, and its 3′ residue is bound to the 5′ molecule of R3. Any stretch of nucleic acid residues denoted by either R1 or R3, which is greater than 1, is preferably a heteropolymer, but can also be a homopolymer. In certain embodiments, n and m are integers between about 1 and 2000, preferably between about 1 and 1000, and preferably between about 1 and 500. In other embodiments, the isolated nucleic acid molecule is at least about 15 nucleotides, preferably at least about 100 nucleotides, more preferably at least about 150 nucleotides, and even more preferably at least about 200 or more nucleotides in length. In still another embodiment, R1 and R3 are both hydrogen.

As appropriate, the isolated nucleic acid molecules of the present invention can be RNA, for example, mRNA, or DNA, such as cDNA and genomic DNA. DNA molecules can be double-stranded or single-stranded; single stranded RNA or DNA can be either the coding, or sense, strand or the non-coding, or antisense, strand. The nucleic acid molecule can include all or a portion of the coding sequence of the genes of the invention. Additionally, the nucleic acid molecule can be fused to a marker sequence, for example, a sequence that encodes a polypeptide to assist in isolation or purification of the polypeptide. Such sequences include, but are not limited to, those which encode a glutathione-S-transferase (GST) fusion protein and those which encode a hemaglutin A (HA) polypeptide marker from influenza.

An “isolated” nucleic acid molecule, as used herein, is one that is separated from nucleic acid which normally flanks the nucleic acid molecule in nature. With regard to genomic DNA, the term “isolated” refers to nucleic acid molecules which are separated from the chromosome with which the genomic DNA is naturally associated. For example, the isolated nucleic acid molecule can contain less than about 5 kb, 4 kb, 3 kb, 2 kb, 1 kb, 0.5 kb or 0.1 kb of nucleotides which flank the nucleic acid molecule in the genomic DNA of the cell from which the nucleic acid is derived.

Moreover, an isolated nucleic acid of the invention, such as a cDNA or RNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated. In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

Further, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention.

The invention further provides variants of the isolated nucleic acid molecules of the invention. Such variants can be naturally occurring, such as allelic variants (same locus), homologs (different locus), and orthologs (different organism), or may be constructed by recombinant DNA methods or by chemical synthesis. Such non-naturally occurring variants can be made using well-known mutagenesis techniques, including those applied to polynucleotides, cells, or organisms. Accordingly, variants can contain nucleotide substitutions, deletions, inversions and/or insertions in either or both the coding and non-coding region of the nucleic acid molecule. Further, the variations can produce both conservative and non-conservative amino acid substitutions.

Typically, variants have a substantial identity with a nucleic acid molecule selected from the group consisting of the sequences disclosed herein and the complements thereof. Particularly preferred are nucleic acid molecules and fragments which have at least about 60%, at least about 70, at least about 80, at least about 85%, at least about 90%, at least about 95%, at least about 96%, at least about 97%, at least about 98%, or at least about 99% or more identity with nucleic acid molecules described herein.

Such nucleic acid molecules can be readily identified as being able to hybridize under stringent conditions to a nucleotide sequence and the complements thereof. In one embodiment, the variants hybridize under high stringency hybridization conditions (e.g., for selective hybridization) to a nucleotide sequence.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45 C, followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Preferably, stringent hybridization conditions are hybridization in 6× sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M Sodium Phosphate, 7% SDS at 65° C., followed by one or more washes at 0.2×SSC, 1% SDS at 65° C.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences. In certain embodiments, the length of a sequence aligned for comparison purposes is at least 30%, preferably at least 40%, more preferably at least 60%, and even more preferably at least 70%, 80% or 90% of the length of the reference sequence. The actual comparison of the two sequences can be accomplished by well-known methods, for example, using a mathematical algorithm. A preferred, non-limiting example of such a mathematical algorithm is described in Karlin et al. (1993) Proc. Natl. Acad. Sci. USA, 90:5873-5877. Such an algorithm is incorporated into the NBLAST and XBLAST programs (version 2.0) as described in Altschul et al. (1997) Nucleic Acids Res., 25:389-3402. When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM 120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci. 10:3-5; and FASTA described in Pearson and Lipman (1988) PNAS, 85:2444-8.

In another embodiment, the percent identity between two amino acid sequences can be accomplished using the GAP program in the CGC software package using either a BLOSUM 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the CGC software package, using a gap weight of 50 and a length weight of 3.

The present invention also provides isolated nucleic acids that contain a fragment or portion that hybridizes under highly stringent conditions to a nucleotide sequence and the complements thereof. In one embodiment, the nucleic acid consists of a portion of a nucleotide sequence and the complements thereof. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, which encode antigenic proteins or polypeptides described herein are useful. Additionally, nucleotide sequences described herein can also be contigged (e.g., overlapped or joined) to produce longer sequences.

In a related aspect, the nucleic acid fragments of the invention are used as probes or primers in assays such as those described herein. “Probes” are oligonucleotides that hybridize in a base-specific manner to a complementary strand of nucleic acid. Such probes include polypeptide nucleic acids, as described in Nielsen et al. (1991) Science, 254, 1497-1500. Typically, a probe comprises a region of nucleotide sequence that hybridizes under highly stringent conditions to at least about 15, typically about 20-25, and more typically about 40, 50 or 75 consecutive nucleotides of a nucleic acid selected from the group consisting of the sequences disclosed herein and the complements thereof. More typically, the probe further comprises a label, e.g., radioisotope, fluorescent compound, enzyme, or enzyme co-factor.

As used herein, the term “primer” refers to a single-stranded oligonucleotide which acts as a point of initiation of template-directed DNA synthesis using well-known methods (e.g., PCR, LCR) including, but not limited to those described herein. The appropriate length of the primer depends on the particular use, but typically ranges from about 15 to 30 nucleotides. The term “primer site” refers to the area of the target DNA to which a primer hybridizes. The term “primer pair” refers to a set of primers including a 5′ (upstream) primer that hybridizes with the 5′ end of the nucleic acid sequence to be amplified and a 3′ (downstream) primer that hybridizes with the complement of the sequence to be amplified.

The nucleic acid molecules of the invention such as those described above can be identified and isolated using standard molecular biology techniques and the sequence information provided in the sequences. For example, nucleic acid molecules can be amplified and isolated by the polymerase chain reaction using synthetic oligonucleotide primers designed based on one or more of the sequences provided in the sequences disclosed herein and the complements thereof. See generally PCR Technology: Principles and Applications for DNA Amplification (ed. H. A. Erlich, Freeman Press, NY, N.Y., 1992); PCR Protocols: A Guide to Methods and Applications (Eds. Innis, et al. Academic Press, San Diego, Calif., 1990); Mattila et al. (1991) Nucleic Acids Res. 19:4967; Eckert et al. (1991) PCR Methods and Applications, 1:17; PCR (eds. McPherson et al. IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace (1989) Genomics, 4:560, Landegren et al. (1988) Science, 241:1077, transcription amplification (Kwoh et al. (1989) Proc. Natl. Acad. Sci. USA, 86:1173), and self-sustained sequence replication (Guatelli et al. (1990) Proc. Nat. Acad. Sci. USA, 87:1874) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be radiolabelled and used as a probe for screening a cDNA library, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a protein of the appropriate molecular weight. For example, the direct analysis of the nucleotide sequence of nucleic acid molecules of the present invention can be accomplished using well-known methods that are commercially available. See, for example, Sambrook et al. Molecular Cloning, A Laboratory Manual (2nd Ed., CSHP, New York 1989); Zyskind et al. Recombinant DNA Laboratory Manual, (Acad. Press, 1988)). Using these or similar methods, the protein(s) and the DNA encoding the protein can be isolated, sequenced and further characterized.

Antisense nucleic acids of the invention can be designed using the nucleotide sequences of the sequences described herein, and constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

Additionally, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry, 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA, 93:14670. PNAs can be further modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra, Finn et al. (1996) Nucleic Acids Res. 24(17):3357-63, Mag et al. (1989) Nucleic Acids Res. 17:5973, and Peterser et al. (1975) Bioorganic Med. Chem. Lett. 5:1119.

The nucleic acid molecules and fragments of the invention can also include other appended groups such as peptides (e.g., for targeting host cell receptors in vivo), or agents facilitating transport across the cell membrane (see, e.g., Letsinger et al. (1989) Proc. Natl. Acad. Sci. USA, 86:6553-6556; Lemaitre et al. (1987) Proc. Natl. Acad. Sci. USA, 84:648-652; PCT Publication No. WO88/0918) or the blood brain barrier (see, e.g., PCT Publication No. WO89/10134). In addition, oligonucleotides can be modified with hybridization-triggered cleavage agents (see, e.g., Krol et al. (1988) Bio-Techniques, 6:958-976) or intercalating agents (see, e.g., Zon (1988) Pharm Res. 5:539-549).

Uses of the nucleic acids of the invention are described in detail in below. In general, the isolated nucleic acid sequences can be used as molecular weight markers on Southern gels, and as chromosome markers which are labeled to map related gene positions. The nucleic acid sequences can also be used to compare with endogenous DNA sequences in patients to identify genetic disorders, and as probes, such as to hybridize and discover related DNA sequences or to subtract out known sequences from a sample. The nucleic acid sequences can further be used to derive primers for genetic fingerprinting, to raise anti-protein antibodies using DNA immunization techniques, and as an antigen to raise anti-DNA antibodies or elicit immune responses. Additionally, the nucleotide sequences of the invention can be used identify and express recombinant proteins for analysis, characterization or therapeutic use, or as markers for tissues in which the corresponding protein is expressed, either constitutively, during tissue differentiation, or in disease states.

### Vectors and Host Cells

Another aspect of the invention pertains to nucleic acid vectors containing a nucleic acid selected from the group consisting of the sequences disclosed herein. These vectors comprise a sequence of the invention has been inserted in a sense or antisense orientation. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene, 67:31-40), pMAL (New England Biolabs, Beverly, Mass.) and pRIT5 (Pharmacia, Piscataway, N.J.) which fuse glutathione 5-transferase (GST), maltose E binding protein, or protein A, respectively, to the target recombinant protein.

Examples of suitable inducible non-fusion E. coli expression vectors include pTrc (Amann et al. (1988) Gene, 69:301-315) and pET 11d (Studier et al. Gene Expression Technology: Methods in Enzymology, 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gni). This viral polymerase is supplied by host strains BL21(DE3) or HMS174(DE3) from a resident prophage harboring a T7 gni gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology, 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari et al. (1987) EMBO J. 6:229-234), pMFa (Kurjan and Herskowitz (1982) Cell 30:933-943), pJRY88 (Schultz et al. (1987) Gene, 54:113-123), pYES2 (Invitrogen Corporation, San Diego, Calif.), and pPicZ (InVitrogen Corp, San Diego, Calif.).

Alternatively, a nucleic acid of the invention can be expressed in insect cells using baculovirus expression vectors. Baculovirus vectors available for expression of proteins in cultured insect cells (e.g., Sf 9 cells) include the pAc series (Smith et al. (1983) Mol. Cell. Biol. 3:2156-2165) and the pVL series (Lucklow and Summers (1989) Virology, 170:31-39).

In yet another embodiment, a nucleic acid of the invention is expressed in mammalian cells using a mammalian expression vector. Examples of mammalian expression vectors include pCDM8 (Seed (1987) Nature, 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al. supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell, 33:729-740; Queen and Baltimore (1983) Cell, 33:741-748), neuron-specific promoters (e.g., the neurofilament promoter; Byrne and Ruddle (1989) Proc. Natl. Acad. Sci. USA, 86:5473-5477), pancreas-specific promoters (Edlund et al. (1985) Science, 230:912-916), and mammary gland-specific promoters (e.g., milk whey promoter; U.S. Pat. No. 4,873,316 and European Application Publication No. 264,166). Developmentally regulated promoters are also encompassed, for example the murine hox promoters (Kessel and Gruss (1990) Science, 249:374-379) and the alpha-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to at least one expression control element in a manner which allows for expression (by transcription of the DNA molecule) of an RNA molecule which is antisense to an mRNA of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Nucleic acid encoding a selectable marker can be introduced into a host cell on the same vector as that nucleic acid of the invention or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid of the invention have been introduced. Such host cells can then be used to create non-human transgenic animals in which exogenous nucleotide sequences have been introduced into their genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a nucleic acid of the invention into the male pronuclei of a fertilized oocyte, e.g., by microinjection, retroviral infection, and allowing the oocyte to develop in a pseudopregnant female foster animal. The sequence can be introduced as a transgene into the genome of a non-human animal. Intronic sequences and polyadenylation signals can also be included in the transgene to increase the efficiency of expression of the transgene. A tissue-specific regulatory sequence(s) can be operably linked to the transgene to direct expression of a polypeptide in particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA in tissues or cells of the animals. A transgenic founder animal can then be used to breed additional animals carrying the transgene. Moreover, transgenic animals carrying a transgene encoding the transgene can further be bred to other transgenic animals carrying other transgenes.

Homologously recombinant host cells can also be produced that allow the in situ alteration of endogenous polynucleotide sequences of the invention in a host cell genome. The host cell includes, but is not limited to, a stable cell line, cell in vivo, or cloned microorganism. This technology is more fully described in WO 93/09222, WO 91/12650, WO 91/06667, U.S. Pat. No. 5,272,071, and U.S. Pat. No. 5,641,670. Briefly, specific polynucleotide sequences corresponding to the polynucleotides or sequences proximal or distal to a gene are allowed to integrate into a host cell genome by homologous recombination where expression of the gene can be affected. In one embodiment, regulatory sequences are introduced that either increase or decrease expression of an endogenous sequence. Accordingly, a protein can be produced in a cell not normally producing it. Alternatively, increased expression of a protein can be effected in a cell normally producing the protein at a specific level. Further, expression can be decreased or eliminated by introducing a specific regulatory sequence. The regulatory sequence can be heterologous to the protein sequence or can be a homologous sequence with a desired mutation that affects expression. Alternatively, the entire gene can be deleted. The regulatory sequence can be specific to the host cell or capable of functioning in more than one cell type. Still further, specific mutations can be introduced into any desired region of the gene to produce mutant proteins of the invention. Such mutations could be introduced, for example, into the specific functional regions.

To create an homologous recombinant animal, a vector is prepared which contains at least a portion of a nucleic acid of the invention into which a deletion, addition or substitution has been introduced to thereby alter, e.g., functionally disrupt, the endogenous gene. In one embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced nucleic acid has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-human animals can be produced which contain selected systems that allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication Nos. WO 97/07668 and WO 97/07669.

### Polypeptides

The present invention also provides isolated polypeptides and variants and fragments thereof that are encoded by the nucleic acid molecules of the invention, especially as shown in SEQ ID NOS:1-34. For example, as described above, the nucleotide sequences can be used to design primers to clone and express cDNAs encoding the polypeptides of the invention. Further, the nucleotide sequences of the invention, e.g., the sequences disclosed herein, can be analyzed using routine search algorithms (e.g., BLAST, Altschul et al. (1990) J. Mol. Biol. 215:403-410; BLAZE, Brutlag et al. (1993) Comp. Chem. 17:203-207) to identify open reading frames (ORFs).

As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell and still be “isolated” or “purified.”

The polypeptides of the invention can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the protein preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide comprises an amino acid sequence encoded by a nucleic acid comprising a nucleotide sequence selected from the group consisting of the sequences disclosed herein and the complements thereof. However, the invention also encompasses sequence variants. Variants include a substantially homologous protein encoded by the same genetic locus in an organism, i.e., an allelic variant. Variants also encompass proteins derived from other genetic loci in an organism, but having substantial homology to a polypeptide encoded by a nucleic acid comprising a nucleotide sequence and the complements thereof. Variants also include proteins substantially homologous to these polypeptides but derived from another organism, i.e., an ortholog. Variants also include proteins that are substantially homologous to these polypeptides that are produced by chemical synthesis. Variants also include proteins that are substantially homologous or identical to these polypeptides that are produced by recombinant methods.

As used herein, two proteins (or a region of the proteins) are substantially homologous or identical when the amino acid sequences are at least about 45-55%, typically at least about 70-75%, more typically at least about 80-85%, and most typically at least about 90-95% or more homologous or identical. A substantially homologous amino acid sequence, according to the present invention, will be encoded by a nucleic acid hybridizing to a nucleic acid sequence selected from the group consisting of the sequences, or portion thereof under stringent conditions as more described above.

To determine the percent homology or identity of two amino acid sequences, or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As used herein, amino acid or nucleic acid “homology” is equivalent to amino acid or nucleic acid “identity”. The percent homology between the two sequences is a function of the number of identical positions shared by the sequences (i.e., percent homology equals the number of identical positions/total number of positions times 100).

The invention also encompasses polypeptides having a lower degree of identity but having sufficient similarity so as to perform one or more of the same functions performed by a polypeptide encoded by a nucleic acid of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al. (1990) Science 247:1306-1310.

TABLE 4
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Preferred computer program methods to determine identify and similarity between two sequences include, but are not limited to, GCG program package (Devereux, J., et al. (1984) Nucleic Acids Res. 12(1):387), BLASTP, BLASTN, FASTA (Atschul, S. F. et al. (1990) J. Molec. Biol. 215:403).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

As indicated, variants can be naturally-occurring or can be made by recombinant means or chemical synthesis to provide useful and novel characteristics for the polypeptide. This includes preventing immunogenicity from pharmaceutical formulations by preventing protein aggregation.

Amino acids that are essential for function can be identified by methods known in the art, such as site-directed mutagenesis or alanine-scanning mutagenesis (Cunningham et al. (1989) Science 244:1081-1085). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al. (1992) J. Mol. Biol. 224:899-904; de Vos et al. (1992) Science 255:306-312).

The invention also includes polypeptide fragments of the polypeptides of the invention. Fragments can be derived from a polypeptide encoded by a nucleic acid comprising a nucleotide sequence selected from the group consisting of the sequences disclosed herein and the complements thereof. However, the invention also encompasses fragments of the variants of the polypeptides described herein.

As used herein, a fragment comprises at least 6 contiguous amino acids. Useful fragments include those that retain one or more of the biological activities of the polypeptide as well as fragments that can be used as an immunogen to generate polypeptide specific antibodies.

Biologically active fragments (peptides which are, for example, 6, 9, 12, 15, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

The invention also provides fragments with immunogenic properties. These contain an epitope-bearing portion of the polypeptides and variants of the invention. These epitope-bearing peptides are useful to raise antibodies that bind specifically to a polypeptide or region or fragment. These peptides can contain at least 6, 7, 8, 9, 12, at least 14, or between at least about 15 to about 30 amino acids. The epitope-bearing peptide and polypeptides may be produced by any conventional means (Houghten (1985) Proc. Natl. Acad. Sci. USA 82:5131-5135). Simultaneous multiple peptide synthesis is described in U.S. Pat. No. 4,631,211.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion proteins. These comprise a polypeptide of the invention operatively linked to a heterologous protein having an amino acid sequence not substantially homologous to the polypeptide. “Operatively linked” indicates that the polypeptide protein and the heterologous protein are fused in-frame. The heterologous protein can be fused to the N-terminus or C-terminus of the polypeptide. In one embodiment the fusion protein does not affect function of the polypeptide per se. For example, the fusion protein can be a GST-fusion protein in which the polypeptide sequences are fused to the C-terminus of the GST sequences. Other types of fusion proteins include, but are not limited to, enzymatic fusion proteins, for example beta-galactosidase fusions, yeast two-hybrid GAL fusions, poly-His fusions and Ig fusions. Such fusion proteins, particularly poly-His fusions, can facilitate the purification of recombinant polypeptide. In certain host cells (e.g., mammalian host cells), expression and/or secretion of a protein can be increased by using a heterologous signal sequence. Therefore, in another embodiment, the fusion protein contains a heterologous signal sequence at its N-terminus.

EP-A-O464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al. (1995) Journal of Molecular Recognition 8:52-58 and Johanson et al. (1995) The Journal of Biological Chemistry 270, 16:9459-9471. Thus, this invention also encompasses soluble fusion proteins containing a polypeptide of the invention and various portions of the constant regions of heavy or light chains of immunoglobulins of various subclass (IgG, IgM, IgA, IgE). Preferred as immunoglobulin is the constant part of the heavy chain of human IgG, particularly IgG1, where fusion takes place at the hinge region. For some uses it is desirable to remove the Fc after the fusion protein has been used for its intended purpose, for example when the fusion protein is to be used as antigen for immunizations. In a particular embodiment, the Fc part can be removed in a simple way by a cleavage sequence that is also incorporated and can be cleaved with factor Xa.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid encoding a polypeptide of the invention can be cloned into such an expression vector such that the fusion moiety is linked in-frame to the polypeptide protein.

The isolated polypeptide can be purified from cells that naturally express it, purified from cells that have been altered to express it (recombinant), or synthesized using known protein synthesis methods.

In one embodiment, the protein is produced by recombinant DNA techniques. For example, a nucleic acid molecule encoding the polypeptide is cloned into an expression vector, the expression vector introduced into a host cell and the protein expressed in the host cell. The protein can then be isolated from the cells by an appropriate purification scheme using standard protein purification techniques.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al., Meth. Enzymol. 182: 626-646 (1990) and Rattan et al. (1992) Ann. N.Y. Acad. Sci. 663:48-62.

As is also well known, polypeptides are not always entirely linear. For instance, polypeptides may be branched as a result of ubiquitination, and they may be circular, with or without branching, generally as a result of post-translation events, including natural processing event and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the amino terminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The same type of modification may be present in the same or varying degree at several sites in a given polypeptide. Also, a given polypeptide may contain more than one type of modification.

Uses of the polypeptides of the invention are described in detail below. In general, polypeptides or proteins of the present invention can be used as a molecular weight marker on SDS-PAGE gels or on molecular sieve gel filtration columns using art-recognized methods. The polypeptides of the present invention can be used to raise antibodies or to elicit an immune response. The polypeptides can also be used as a reagent, e.g., a labeled reagent, in assays to quantitatively determine levels of the protein or a molecule to which it binds (e.g., a receptor or a ligand) in biological fluids. The polypeptides can also be used as markers for tissues in which the corresponding protein is preferentially expressed, either constitutively, during tissue differentiation, or in a diseased state. The polypeptides can be used to isolate a corresponding binding partner, e.g., receptor or ligand, such as, for example, in an interaction trap assay, and to screen for peptide or small molecule antagonists or agonists of the binding interaction.

### Antibodies

In another aspect, the invention provides antibodies to the polypeptides and polypeptide fragments of the invention, e.g., having an amino acid encoded by a nucleic acid comprising all or a portion of a nucleotide sequence selected from the group consisting of the sequences disclosed herein. The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)2 fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al. (1977) Nature 266:55052; R. H. Kenneth, in Monoclonal Antibodies: A New Dimension In Biological Analyses, Plenum Publishing Corp., New York, N.Y. (1980); and Lerner (1981) Yale J. Biol. Med. 54:387-402. Moreover, the ordinarily skilled worker will appreciate that there are many variations of such methods that also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a polypeptide of the invention, e.g., using a standard ELISA assay.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93. For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies that recognize a selected epitope can be generated using a technique referred to as “guided selection.” This technology is described, for example, in Jespers et al. (1994) Bio/technology 12:899-903).

Uses of the antibodies of the invention are described in detail below. In general, antibodies of the invention (e.g., a monoclonal antibody) can be used to isolate a polypeptide of the invention by standard techniques, such as affinity chromatography or immunoprecipitation. A polypeptide specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, (-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

### Computer Readable Means

The nucleotide or amino acid sequences of the invention are also provided in a variety of mediums to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exists in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and MicroSoft Word, or represented in the form of an ASCII file, stored in a database application, such as DB2, Sybase, Oracle, or the like. The skilled artisan can readily adapt any number of dataprocessor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTX (NCBIA).

For example, software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

### Detection Assays

Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the nucleic acid (or a portion of the sequence) has been isolated, it can be used to map the location of the gene on a chromosome. The mapping of the sequences to chromosomes is an important first step in correlating these sequences with genes associated with disease. Briefly, genes can be mapped to chromosomes by preparing PCR primers (preferably 15-25 by in length) from the nucleic acid molecules described herein. Computer analysis of the sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the appropriate nucleotide sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycle. Using the nucleic acid molecules of the invention to design oligonucleotide primers, sublocalization can be achieved with panels of fragments from specific chromosomes. Other mapping strategies which can similarly be used to map a specified sequence to its chromosome include in situ hybridization (described in Fan et al. (1990) PNAS 97:6223-27), pre-screening with labeled flow-sorted chromosomes, and pre-selection by hybridization to chromosome specific cDNA libraries.

Fluorescence in situ hybridization (FISH) of a nucleotide sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a nucleotide sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. for a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Medelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland et al. (1987) Nature 325:783-787.

Moreover, differences in the DNA sequences between individuals affected and unaffected with a disease associated with a specified gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible form chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The nucleotide sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique that determines the actual base-by-base DNA sequence of selected portions of an individual's genome. Thus, the nucleic acid molecules described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The nucleic acid molecules of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of these sequences can comfortably provide positive individual identification with a panel of perhaps 10 to 1,000 primers which each yield a noncoding amplified sequence of 100 bases. If predicted coding sequences are used, a more appropriate number of primers for positive individual identification would be 500-2,000.

If a panel of reagents from nucleic acid molecules described herein is used to generate a unique identification database for an individual, those same reagents can later be used to identify tissue from that individual. Using the unique identification database, positive identification of the individual, living or dead, can be made from extremely small tissue samples.

3. Use of Partial Sequences in Forensic Biology

DNA-based identification techniques can also be used in forensic biology. Forensic biology is a scientific field employing genetic typing of biological evidence found at a crime scene as a means of positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of sequences described herein are particularly appropriate for this use, as greater numbers of polymorphisms occur in the noncoding regions, making it easier to differentiate individuals using this technique. Examples of polynucleotide reagents include the nucleic acid molecules or the invention, or portions thereof, e.g., fragments having a length of at least 20 bases, preferably at least 30 bases.

The nucleic acid molecules described herein can further be used to provide polynucleotide reagents, e.g., labeled or labelable probes which can be used in, or example, an in situ hybridization technique, to identify a specific tissue. This can be very useful in cases where a forensic pathologist is presented with a tissue of unknown origin. Panels of such probes can be used to identify tissue by species and/or by organ type.

In a similar fashion, these reagents, primers or probes can be used to screen tissue culture for contamination (i.e., screen for the presence of a mixture of different types of cells in a culture).

### Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining protein and/or nucleic acid expression as well as activity of proteins of the invention, in the context of a biological sample (e.g., blood, serum, cells, tissue) to thereby determine whether an individual is afflicted with a disease or disorder, or is at risk of developing a disorder, associated with aberrant expression or activity. The invention also provides for prognostic (or predictive) assays for determining whether an individual is at risk of developing a disorder associated with activity or expression of proteins or nucleic acids of the invention.

Disorders relating to programmed cell death are particularly relevant as discussed in detail herein below.

For example, mutations in a specified gene can be assayed in a biological sample. Such assays can be used for prognostic or predictive purpose to thereby prophylactically treat an individual prior to the onset of a disorder characterized by or associated with expression or activity of nucleic acid molecules or proteins of the invention.

Another aspect of the invention pertains to monitoring the influence of agents (e.g., drugs, compounds) on the expression or activity of proteins of the invention in clinical trials.

These and other agents are described in further detail in the following sections.

1. Diagnostic Assays

An exemplary method for detecting the presence or absence of proteins or nucleic acids of the invention in a biological sample involves obtaining a biological sample from a test subject and contacting the biological sample with a compound or an agent capable of detecting the protein, or nucleic acid (e.g., mRNA, genomic DNA) that encodes the protein, such that the presence of the protein or nucleic acid is detected in the biological sample. A preferred agent for detecting mRNA or genomic DNA is a labeled nucleic acid probe capable of hybridizing to mRNA or genomic DNA sequences described herein. The nucleic acid probe can be, for example, a full-length nucleic acid, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of the sequences disclosed herein, or the complement of the sequences disclosed herein, or a portion thereof. Other suitable probes for use in the diagnostic assays of the invention are described herein.

In one embodiment, the agent for detecting proteins of the invention is an antibody capable of binding to the protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, calls and biological fluids isolated from a subject, as well as tissues, cells and fluids present within a subject. That is, the detection method of the invention can be used to detect mRNA, protein, or genomic DNA of the invention in a biological sample in vitro as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled anti-protein antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample or biopsy isolated by conventional means from a subject.

In another embodiment, the methods further involve obtaining a control biological sample from a control subject, contacting the control sample with a compound or agent capable of detecting protein, mRNA, or genomic DNA of the invention, such that the presence of protein, mRNA or genomic DNA is detected in the biological sample, and comparing the presence of protein, mRNA or genomic DNA in the control sample with the presence of protein, mRNA or genomic DNA in the test sample.

The invention also encompasses kits for detecting the presence of proteins or nucleic acid molecules of the invention in a biological sample. For example, the kit can comprise a labeled compound or agent capable of detecting protein or mRNA in a biological sample; means for determining the amount of in the sample; and means for comparing the amount of in the sample with a standard. The compound or agent can be packaged in a suitable container. The kit can further comprise instructions for using the kit to detect protein or nucleic acid.

2. Prognostic Assays

The diagnostic methods described herein can furthermore be utilized to identify subjects having or at risk of developing a disease or disorder associated with aberrant expression or activity of proteins and nucleic acid molecules of the invention. Accordingly, the term “diagnostic” refers not only to ascertaining whether a subject has an active disease but also relates to ascertaining whether a subject is predisposed to developing active disease as well as ascertaining the probability that treatment of active disease will be effective. For example, the assays described herein, such as the preceding diagnostic assays or the following assays can be utilized to identify a subject having or at risk of developing a disorder associated with protein or nucleic acid expression or activity such as a proliferative disorder, a differentiative or developmental disorder, or a hematopoietic disorder. Alternatively, the prognostic assays can be utilized to identify a subject having or at risk for developing a differentiative or proliferative disease (e.g., cancer). Thus, the present invention provides a method for identifying a disease or disorder associated with aberrant expression or activity of proteins or nucleic acid molecules of the invention, in which a test sample is obtained from a subject and protein or nucleic acid (e.g., mRNA, genomic DNA) is detected, wherein the presence of protein or nucleic acid is diagnostic for a subject having or at risk of developing a disease or disorder associated with aberrant expression or activity of the protein or nucleic acid sequence of the invention. As used herein, a “test sample” refers to a biological sample obtained from a subject of interest. For example, a test sample can be a biological fluid (e.g., serum), cell or tissue sample.

Disorders relating to programmed cell death are particularly relevant as discussed in detail herein below.

Furthermore, the prognostic assays described herein can be used to determine whether a subject can be administered an agent (e.g., an agonist, antagonist, peptidomimetic, protein, polypeptide, nucleic acid, small molecule, or other drug candidate) to treat a disease or disorder associated with aberrant expression or activity of a protein or nucleic acid molecule of the invention. For example, such methods can be used to determine whether a subject can be effectively treated with an agent for a disorder, such as a proliferative disorder, a differentiative or a developmental disorder. Alternatively, such methods can be used to determine whether a subject can be effectively treated with an agent for a differentiative or proliferative disease (e.g., cancer). Thus, the present invention provides methods for determining whether a subject can be effectively treated with an agent for a disorder associated with aberrant expression or activity of a protein or nucleic acid of the present invention, in which a test sample is obtained and protein or nucleic acid expression or activity is detected (e.g., wherein the abundance of particular protein or nucleic acid expression or activity is diagnostic for a subject that can be administered the agent to treat a disorder associated with aberrant expression or activity.)

Disorders relating to programmed cell death are particularly relevant as discussed in detail herein below.

The methods of the invention can also be used to detect genetic alterations in genes or nucleic acid molecules of the present invention, thereby determining if a subject with the altered gene is at risk for a disorder characterized by aberrant development, aberrant cellular differentiation, aberrant cellular proliferation or an aberrant hematopoietic response. In certain embodiments, the methods include detecting, in a sample of cells from the subject, the presence or absence of a genetic alteration characterized by at least one of an alteration affecting the integrity of a gene encoding a particular protein, or the mis-expression of the gene. For example, such genetic alterations can be detected by ascertaining the existence of at least one of (1) a deletion of one or more nucleotides; (2) an addition of one or more nucleotides; (3) a substitution of one or more nucleotides, (4) a chromosomal rearrangement; (5) an alteration in the level of a messenger RNA transcript; (6) aberrant modification, such as of the methylation pattern of the genomic DNA; (7) the presence of a non-wild type splicing pattern of a messenger RNA transcript; (8) a non-wild type level; (9) allelic loss; and (10) inappropriate post-translational modification. As described herein, there are a large number of assay techniques known in the art that can be used for detecting alterations in a particular gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such an anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh et al., (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a given gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicate mutations in the sample DNA. Moreover, the use of sequence specific ribozymes (see, for sample, U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotide probes (Cronin et al. (1996) Human Mutation 7:244-255; Kozal et al. (1996) Nature Medicine 2:753-759). For example, genetic mutations can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the gene and detect mutations by comparing the sequence of the gene from the sample with the corresponding wild-type (control) gene sequence. Examples of sequencing reactions include those based on techniques developed by Maxim and Gilbert ((1997) PNAS 74:560) or Sanger ((1977) PNAS 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type sequence with potentially mutant RNA or DNA obtained from a tissue sample. The double-standard duplexes are treated with an agent that cleaves single-stranded regions of the duplex such as which will exist due to base pair mismatches between the control and sample strands. For instance, RNA/DNA duplexes can be treated with Rnase and DNA/DNA hybrids treated with 51 nuclease to enzymatically digest the mismatched regions. After digestion of the mismatched regions, the resulting material is then separated by size on denaturing polyacrylamide gels to determine the site of mutation. See, for example Cotton et al. (1988) Proc. Natl. Acad. Sci. USA 85:4397; Saleeba et al. (1992) Methods Enzymol. 217:286-295. In certain embodiments, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an nucleotide sequence of the invention is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat Res 285:125-144; and Hayashi (1992) Genet Anal. Tech. Appl. 9:73-79). Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6320). Such allele-specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell. Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene of the present invention. Any cell type or tissue in which the gene is expressed may be utilized in the prognostic assays described herein.

3. Monitoring of Effects During Clinical Trials

Monitoring the influence of agents (e.g., drugs, compounds) on the expression or activity of nucleic acid molecules or proteins of the present invention (e.g., modulation of cellular signal transduction, regulation of gene transcription in a cell involved in development or differentiation, regulation of cellular proliferation) can be applied not only in basic drug screening, but also in clinical trials. For example, the effectiveness of an agent determined by a screening assay as described herein to increase gene expression, protein levels, or upregulate protein activity, can be monitored in clinical trials of subjects exhibiting decreased gene expression, protein levels, or downregulated protein activity. Alternatively, the effectiveness of an agent determined by a screening assay to decrease gene expression, protein levels, or downregulate protein activity, can be monitored in clinical trials of subjects exhibiting increased gene expression, protein levels, or upregulated protein activity. In such clinical trials, the expression or activity of the specified gene and, preferably, other genes that have been implicated in, for example, a proliferative disorder can be used as a “read out” or markers of the phenotype of a particular cell.

For example, and not by way of limitation, genes that are modulated in cells by treatment with an agent (e.g., compound, drug or small molecule) which modulates protein activity (e.g., identified in a screening assay as described herein) can be identified. Thus, to study the effect of agents on proliferative disorders, developmental or differentiative disorder, or hematopoietic disorder, for example, in a clinical trial, cells can be isolated and RNA prepared and analyzed for the levels of expression of the specified gene and other genes implicated in the proliferative disorder, developmental or differentiative disorder, or hematopoietic disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of the specified gene or other genes. In this way, the gene expression pattern can serve as a marker, indicative of the physiological response of the cells to the agent. Accordingly, this response state may be determined before, and at various points during, treatment of the individual with the agent.

Disorders relating to programmed cell death are particularly relevant as discussed in detail herein below.

In one embodiment, the present invention provides a method for monitoring the effectiveness of treatment of a subject with an agent (e.g., an agonist, antagonist, peptidomimetic, protein, polypeptide, nucleic acid, small molecule, or other drug candidate identified by the screening assays described herein) comprising the steps of (i) obtaining a pre-administration sample from a subject prior to administration of the agent; (ii) detecting the level of expression of a specified protein, mRNA, or genomic DNA of the invention in the pre-administration sample; (iii) obtaining one or more post-administration samples from the subject; (iv) detecting the level of expression or activity of the protein, mRNA, or genomic DNA in the post-administration samples; (v) comparing the level of expression or activity of the protein, mRNA, or genomic DNA in the pre-administration sample with the protein, mRNA, or genomic DNA in the post-administration sample or samples; and (vi) altering the administration of the agent to the subject accordingly. For example, increased administration of the agent may be desirable to increase the expression or activity of the protein or nucleic acid molecule to higher levels than detected, i.e., to increase effectiveness of the agent. Alternatively, decreased administration of the agent may be desirable to decrease effectiveness of the agent. According to such an embodiment, protein or nucleic acid expression or activity may be used as an indicator of the effectiveness of an agent, even in the absence of an observable phenotypic response.

### Screening Assays

The invention provides a method (also referred to herein as a “screening assay”) for identifying modulators, i.e., candidate or test compounds or agents (e.g., antisense, polypeptides, peptidomimetics, small molecules or other drugs) which bind to nucleic acid molecules, polypeptides or proteins described herein or have a stimulatory or inhibitory effect on, for example, expression or activity of the nucleic acid molecules, polypeptides or proteins of the invention.

As an example, apoptosis-specific assays may be used to identify modulators of any of the target nucleic acids or proteins of the present invention, which proteins and/or nucleic acids are related to apoptosis. Accordingly, an agent that modulates the level or activity of any of these nucleic acids or proteins can be identified by means of apoptosis-specific assays. For example, high throughput screens exist to identify apoptotic cells by the use of chromatin or cytoplasmic-specific dyes. Thus, hallmarks of apoptosis, cytoplasmic condensation and chromosome fragmentation, can be used as a marker to identify modulators of any of the genes related to programmed-cell death described herein. Other assays include, but are not limited to, the activation of specific endogenous proteases, loss of mitochondrial function, cytoskeletal disruption, cell shrinkage, membrane blebbing, and nuclear condensation due to degradation of DNA.

In one embodiment, the invention provides assays for screening candidate or test compounds that bind to or modulate the activity of protein or polypeptide described herein or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. U.S.A. 90:6909; Erb et al. (1994) Proc. Natl. Acad. Sci. U.S.A. 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. U.S.A. 89:1865-1869) or on phage (Scott and Smith (1990) Science 249:386-390); (Devlin (1990) Science 249:404-406); (Cwirla et al. (1990) Proc. Natl. Acad. Sci. 97:6378-6382); (Felici (1991) J. Mol. Biol. 222:301-310); (Ladner supra).

In one embodiment, an assay is a cell-based assay in which a cell that expresses an encoded polypeptide (e.g., cell surface protein such as a receptor) is contacted with a test compound and the ability of the test compound to bind to the polypeptide is determined. The cell, for example, can be of mammalian origin, such as a keratinocyte. Determining the ability of the test compound to bind to the polypeptide can be accomplished, for example, by coupling the test compound with a radioisotope or enzymatic label such that binding of the test compound to the polypeptide can be determined by detecting the labeled with 125I, 35S, 14C, or 3H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with the polypeptide without the labeling of any of the interactants. For example, a microphysiometer can be used to detect the interaction of a test compound with the polypeptide without the labeling of either the test compound or the polypeptide. McConnell et al. (1992) Science 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide.

In one embodiment, the assay comprises contacting a cell which expresses an encoded protein described herein on the cell surface (e.g., a receptor) with a polypeptide ligand or biologically-active portion thereof, to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide as compared to the ability of the ligand, or a biologically active portion thereof, to bind to the polypeptide.

In another embodiment, an assay is a cell-based assay comprising contacting a cell expressing a particular target molecule described herein with a test compound and determining the ability of the test compound to modulate or alter (e.g. stimulate or inhibit) the activity of the target molecule. Determining the ability of the test compound to modulate the activity of the target molecule can be accomplished, for example, by determining the ability of a known ligand to bind to or interact with the target molecule. Determining the ability of the known ligand to bind to or interact with the target molecule can be accomplished by one of the methods described above for determining direct binding. In one embodiment, determining the ability of the known ligand to bind to or interact with the target molecule can be accomplished by determining the activity of the target molecule. For example, the activity of the target molecule can be determined by detecting induction of a cellular second messenger of the target (e.g., intracellular Ca2+, diacylglycerol, IP3, etc.), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a cellular response, for example, development, differentiation or rate of proliferation.

In yet another embodiment, an assay of the present invention is a cell-free assay in which protein of the invention or biologically active portion thereof is contacted with a test compound and the ability of the test compound to bind to the protein or biologically active portion thereof is determined. Binding of the test compound to the protein can be determined either directly or indirectly as described above. In one embodiment, the assay includes contacting the protein or biologically active portion thereof with a known compound which binds the protein to form an assay mixture, contacting the assay mixture with a test compound, and determining the ability of the test compound to interact with the protein. Determining the ability of the test compound to interact with the protein comprises determining the ability of the test compound to preferentially bind to the protein or biologically active portion thereof as compared to the known compound.

In another embodiment, the assay is a cell-free assay in which a protein of the invention or biologically active portion thereof is contacted with a test compound and the ability of the test compound to modulate or alter (e.g., stimulate or inhibit) the activity of the protein or biologically active portion thereof is determined. Determining the ability of the test compound to modulate the activity of the protein can be accomplished, for example, by determining the ability of the protein to bind to a known target molecule by one of the methods described above for determining direct binding. Determining the ability of the protein to bind to a target molecule can also be accomplished using a technology such as real-time Bimolecular Interaction Analysis (BIA). Sjolander and Urbaniczky (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

In an alternative embodiment, determining the ability of the test compound to modulate the activity of a protein of the invention can be accomplished by determining the ability of the protein to further modulate the activity of a target molecule. For example, the catalytic/enzymatic activity of the target molecule on an appropriate substrate can be determined as previously described.

In yet another embodiment, the cell-free assay involves contacting a protein of the invention or biologically active portion thereof with a known compound which binds the protein to form an assay mixture, contacting the assay mixture with a test compound, and determining the ability of the test compound to interact with the protein, wherein determining the ability of the test compound to interact with the protein comprises determining the ability of the protein to preferentially bind to or modulate the activity of a target molecule.

The cell-free assays of the present invention are amenable to use of both soluble and/or membrane-bound forms of isolated proteins. In the case of cell-free assays in which a membrane-bound form an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton®X-100, Triton® X-114, Thesit®, Isotridecypoly(ethylene glycol ether)n,3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

In more than one embodiment of the above assay methods of the present invention, it may be desirable to immobilize either the protein or its target molecule to facilitate separation of complexed from uncomplexed forms of one or both of the proteins, as well as to accommodate automation of the assay. Binding of a test compound to the protein, or interaction of the protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or protein of the invention, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, either a protein of the invention or a target molecule can be immobilized utilizing conjugation of biotin and streptavidin. Biotinylated protein of the invention or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a protein of the invention or target molecules, but which do not interfere with binding of the protein to its target molecule, can be derivatized to the wells of the plate, and unbound target or protein trapped in the wells by antibody conjugation. Methods for detecting such complexes, in addition to those described above for the GST-immobilized complexes, include immunodetection of complexes using antibodies reactive with the protein or target molecule, as well as enzyme-linked assays which rely on detecting an enzymatic activity associated with the protein or target molecule.

In another embodiment, modulators of expression of nucleic acid molecules of the invention are identified in a method wherein a cell is contacted with a candidate compound and the expression of appropriate mRNA or protein in the cell is determined. The level of expression of appropriate mRNA or protein in the presence of the candidate compound is compared to the level of expression of mRNA or protein in the absence of the candidate compound. The candidate compound can then be identified as a modulator of expression based on this comparison. For example, when expression of mRNA or protein is greater (statistically significantly greater) in the presence of the candidate compound than in its absence, the candidate compound is identified as a stimulator or enhancer of the mRNA or protein expression. Alternatively, when expression of the mRNA or protein is less (statistically significantly less) in the presence of the candidate compound than in its absence, the candidate compound is identified as an inhibitor of the mRNA or protein expression. The level of mRNA or protein expression in the cells can be determined by methods described herein for detecting mRNA or protein.

In yet another aspect of the invention, the proteins of the invention can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins (captured proteins) which bind to or interact with the proteins of the invention and modulate their activity. Such captured proteins are also likely to be involved in the propagation of signals by the proteins of the invention as, for example, downstream elements of a protein-mediated signaling pathway. Alternatively, such captured proteins are likely to be cell-surface molecules associated with non-protein-expressing cells, wherein such captured proteins are involved in signal transduction.

This invention further pertains to novel agents identified by the above-described screening assays. Accordingly, it is within the scope of this invention to further use an agent identified as described herein in an appropriate animal model. For example, an agent identified as described herein (e.g., a modulating agent, an antisense nucleic acid molecule, a specific antibody, or a protein-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

### Methods of Treatment

The present invention provides for both prophylactic and therapeutic methods of treating a subject at risk of (or susceptible to) a disorder or having a disorder associated with aberrant expression or activity of or related to proteins or nucleic acids of the invention. Methods of treatment involve modulating nucleic acid or polypeptide level or activity in a subject having a disorder that can be treated by such modulation. Accordingly, modulation can cause up regulation or down regulation of the levels of expression or up regulation or down regulation of the activity of the nucleic acid or protein. Disorders relating to programmed cell death are particularly relevant as discussed in detail herein below.

Expression of the nucleic acids of the invention has been shown for the following tissues: testes, brain, heart, kidney, skeletal muscle, spleen, lung, smooth muscle, pancreas, and liver. Accordingly, disorders to which the methods disclosed herein are particularly relevant include those involving these tissues.

Disorders involving the spleen include, but are not limited to, splenomegaly, including nonspecific acute splenitis, congestive spenomegaly, and spenic infarcts; neoplasms, congenital anomalies, and rupture. Disorders associated with splenomegaly include infections, such as nonspecific splenitis, infectious mononucleosis, tuberculosis, typhoid fever, brucellosis, cytomegalovirus, syphilis, malaria, histoplasmosis, toxoplasmosis, kala-azar, trypanosomiasis, schistosomiasis, leishmaniasis, and echinococcosis; congestive states related to partial hypertension, such as cirrhosis of the liver, portal or splenic vein thrombosis, and cardiac failure; lymphohematogenous disorders, such as Hodgkin disease, non-Hodgkin lymphomas/leukemia, multiple myeloma, myeloproliferative disorders, hemolytic anemias, and thrombocytopenic purpura; immunologic-inflammatory conditions, such as rheumatoid arthritis and systemic lupus erythematosus; storage diseases such as Gaucher disease, Niemann-Pick disease, and mucopolysaccharidoses; and other conditions, such as amyloidosis, primary neoplasms and cysts, and secondary neoplasms.

Disorders involving the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, al-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver metastatic tumors, and liver fibrosis.

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B1) deficiency and vitamin B12 deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

Disorders involving the heart, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, and disorders involving cardiac transplantation.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schönlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypernephroma, adenocarcinoma of kidney), which includes urothelial carcinomas of renal pelvis.

Disorders involving the testis and epididymis include, but are not limited to, congenital anomalies such as cryptorchidism, regressive changes such as atrophy, inflammations such as nonspecific epididymitis and orchitis, granulomatous (autoimmune) orchitis, and specific inflammations including, but not limited to, gonorrhea, mumps, tuberculosis, and syphilis, vascular disturbances including torsion, testicular tumors including germ cell tumors that include, but are not limited to, seminoma, spermatocytic seminoma, embryonal carcinoma, yolk sac tumor choriocarcinoma, teratoma, and mixed tumors, tumore of sex cord-gonadal stroma including, but not limited to, leydig (interstitial) cell tumors and sertoli cell tumors (androblastoma), and testicular lymphoma, and miscellaneous lesions of tunica vaginalis.

Disorders involving the skeletal muscle include tumors such as rhabdomyosarcoma.

Disorders involving the pancreas include those of the exocrine pancreas such as congenital anomalies, including but not limited to, ectopic pancreas; pancreatitis, including but not limited to, acute pancreatitis; cysts, including but not limited to, pseudocysts; tumors, including but not limited to, cystic tumors and carcinoma of the pancreas; and disorders of the endocrine pancreas such as, diabetes mellitus; islet cell tumors, including but not limited to, insulinomas, gastrinomas, and other rare islet cell tumors.

Preferred disorders include those involving the central nervous system and particularly the brain.

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with the molecules of the present invention or modulators according to that individual's drug response genotype. Pharmacogenomics allows a clinician or physician to target prophylactic or therapeutic treatments to patients who will most benefit from the treatment and to avoid treatment of patients who will experience toxic drug related side effects.

1. Prophylactic Methods

In one aspect, the invention provides a method for preventing in a subject, a disease or condition associated with aberrant expression or activity of genes or proteins of the present invention, by administering to the subject an agent which modulates expression or at least one activity of a gene or protein of the invention. Subjects at risk for a disease that is caused or contributed to by aberrant gene expression or protein activity can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

2. Therapeutic Methods

Another aspect of the invention pertains to methods of modulating expression or activity of genes or proteins of the invention for therapeutic purposes. The modulatory method of the invention involves contacting a cell with an agent that modulates one or more of the activities of the specified protein associated with the cell. An agent that modulates protein activity can be an agent as described herein, such as a nucleic acid or a protein, a naturally-occurring target molecule of a protein described herein, a polypeptide, a peptidomimetic, or other small molecule. In one embodiment, the agent stimulates one or more protein activities. Examples of such stimulatory agents include active protein as well as a nucleic acid molecule encoding the protein that has been introduced into the cell. In another embodiment, the agent inhibits one or more protein activities. Examples of such inhibitory agents include antisense nucleic acid molecules and anti-protein antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a protein or nucleic acid molecule of the invention. In one embodiment, the method involves administering an agent (e.g., an agent identified by a screening assay described herein), or combination of agents that modulates (e.g., upregulates or downregulates) expression or activity of a gene or protein of the invention. In another embodiment, the method involves administering a protein or nucleic acid molecule of the invention as therapy to compensate for reduced or aberrant expression or activity of the protein or nucleic acid molecule.

Stimulation of protein activity is desirable in situations in which the protein is abnormally downregulated and/or in which increased protein activity is likely to have a beneficial effect. Likewise, inhibition of protein activity is desirable in situations in which the protein is abnormally upregulated and/or in which decreased protein activity is likely to have a beneficial effect. One example of such a situation is where a subject has a disorder characterized by aberrant development or cellular differentiation. Another example of such a situation is where the subject has a proliferative disease (e.g., cancer) or a disorder characterized by an aberrant hematopoietic response. Yet another example of such a situation is where it is desirable to achieve tissue regeneration in a subject (e.g., where a subject has undergone brain or spinal cord injury and it is desirable to regenerate neuronal tissue in a regulated manner).

### Pharmaceutical Compositions

The nucleic acid molecules, protein modulators of the protein, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier.

The term “administer” is used in its broadest sense and includes any method of introducing the compositions of the present invention into a subject. This includes producing polypeptides or polynucleotides in vivo as by transcription or translation, in vivo, of polynucleotides that have been exogenously introduced into a subject. Thus, polypeptides or nucleic acids produced in the subject from the exogenous compositions are encompassed in the term “administer.”

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a ubiquitin protease protein or anti-ubiquitin protease antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (I.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

3. Pharmacogenomics

The molecules of the present invention, as well as agents, or modulators which have a stimulatory or inhibitory effect on the protein activity (e.g., gene expression) as identified by a screening assay described herein can be administered to individuals to treat (prophylactically or therapeutically) disorders (e.g., proliferative or developmental disorders) associated with aberrant protein activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a molecule of the invention or modulator thereof, as well as tailoring the dosage and/or therapeutic regimen of treatment with such a molecule or modulator.

Pharmacogenomics deals with clinically significant hereditary variations in the response to drugs due to altered drug disposition and abnormal action in affected persons. See e.g., Eichelbaum (1996) Clin Exp. Pharmacol. Physiol. 23(10-11):983-985 and Linder (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1,000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a protein or a polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2(NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme is the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a molecule or modulator of the present invention) can given an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a molecule or modulator of the invention, such as a modulator identified by one of the exemplary screening assays described herein.

Disorders which may be treated or diagnosed by methods described herein include, but are not limited to disorders involving apoptosis. Certain disorders are associated with an increased number of surviving cells, which are produced and continue to survive or proliferate when apoptosis is inhibited.

As used herein, “programmed cell death” refers to a genetically regulated process involved in the normal development of multicellular organisms. This process occurs in cells destined for removal in a variety of normal situations, including larval development of the nematode C. elegans, insect metamorphosis, development in mammalian embryos, including the nephrogenic zone in the developing kidney, and regression or atrophy (e.g., in the prostate after castration). Programmed cell death can occur following the withdrawal of growth and trophic factors in many cells, nutritional deprivation, hormone treatment, ultraviolet irradiation, and exposure to toxic and infectious agents including reactive oxygen species and phosphatase inhibitors, e.g., okadaic acid, calcium ionophores, and a number of cancer chemotherapeutic agents. See Wilson (1998) Biochem. Cell Biol. 76:573-582 and Hetts (1998) JAMA 279:300-307, the contents of which are incorporated herein by reference. Thus, the proteins of the invention, by being differentially expressed during programmed cell death, e.g., neuronal programmed cell death, can modulate a programmed cell death pathway activity and provide novel diagnostic targets and therapeutic agents for disorders characterized by deregulated programmed cell death, particularly in cells that express the protein.

As used herein, a “disorder characterized by deregulated programmed cell death” refers to a disorder, disease or condition which is characterized by a deregulation, e.g., an upregulation or a downregulation, of programmed cell death. Programmed cell death deregulation can lead to deregulation of cellular proliferation and/or cell cycle progression. Examples of disorders characterized by deregulated programmed cell death include, but are not limited to, neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementias; myelodysplastic syndromes, e.g., aplastic anemia; ischemic injury, e.g., myocardial infarction, stroke, or reperfusion injury; autoimmune disorders, e.g., systemic lupus erythematosus, or immune-mediated glomerulonephritis; or profilerative disorders, e.g., cancer, such as follicular lymphomas, carcinomas with p53 mutations, or hormone-dependent tumors, e.g., breast cancer, prostate cancer, or ovarian cancer). Clinical manifestations of faulty apoptosis are also seen in stroke and in rheumatoid arthritis. Wilson (1998) Biochem. Cell. Biol. 76:573-582.

Failure to remove autoimmune cells that arise during development or that develop as a result of somatic mutation during an immune response can result in autoimmune disease. One of the molecules that plays a critical role in regulating cell death in lymphocytes is the cell surface receptor for Fas.

Viral infections, such as those caused by herpesviruses, poxviruses, and adenoviruses, may result in aberrant apoptosis. Populations of cells are often depleted in the event of viral infection, with perhaps the most dramatic example being the cell depletion caused by the human immunodeficiency virus (HIV). Most T cells that die during HIV infections do not appear to be infected with HIV. Stimulation of the CD4 receptor may result in the enhanced susceptibility of uninfected T cells to undergo apoptosis.

Many disorders can be classified based on whether they are associated with abnormally high or abnormally low apoptosis. Thompson (1995) Science 267:1456-1462. Apoptosis may be involved in acute trauma, myocardial infarction, stroke, and infectious diseases, such as viral hepatitis and acquired immunodeficiency syndrome.

Primary apoptosis deficiencies include graft rejection. Accordingly, the invention is relevant to the identification of genes useful in inhibiting graft rejection.

Primary apoptosis deficiencies also include autoimmune diabetes. Accordingly, the invention is relevant to the identification of genes involved in autoimmune diabetes and accordingly, to the identification of agents that act on these targets to modulate the expression of these genes and hence, to treat or diagnose this disorder. Further, it has been suggested that all autoimmune disorders can be viewed as primary deficiencies of apoptosis (Hetts, above). Accordingly, the invention is relevant for screening for gene expression and transcriptional profiling in any autoimmune disorder and for screening for agents that affect the expression or transcriptional profile of these genes.

Primary apoptosis deficiencies also include local self reactive disorder. This includes Hashimoto thyroiditis.

Primary apoptosis deficiencies also include lymphoproliferation and autoimmunity. This includes, but is not limited to, Canale-Smith syndrome.

Primary apoptosis deficiencies also include cancer. For example, p53 induces apoptosis by acting as a transcription factor that activates expression of various apoptosis-mediating genes or by upregulating apoptosis-mediating genes such as Bax.

Primary apoptosis excesses are associated with neurodegenerative disorders including Alzheimer's disease, Parkinson's disease, spinal muscular atrophy, and amyotrophic lateral sclerosis.

Primary apoptosis excesses are also associated with heart disease including idiopathic dilated cardiomyopathy, ischemic cardiomyopathy, and valvular heart disease. Evidence has also been shown of apoptosis in heart failure resulting from arrhythmogenic right ventricular dysplasia. For all these disorders, see Hetts, above.

Death receptors also include the TNF receptor-1 and hence, TNF acts as a death ligand.

A wide variety of neurological diseases are characterized by the gradual loss of specific sets of neurons. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death.

In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow.

These disorders could result from the activation of genes that promote apoptosis, acquired deficiencies in stromal cells or hematopoietic survival factors, or the direct effects of toxins and mediators of immune responses.

Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis.

The invention also pertains to disorders of the central nervous system (CNS). These disorders include, but are not limited to cognitive and neurodegenerative disorders such as Alzheimer's disease, senile dementia, Huntington's disease, amyotrophic lateral sclerosis, and Parkinson's disease, as well as Gilles de la Tourette's syndrome, autonomic function disorders such as hypertension and sleep disorders, and neuropsychiatric disorders that include, but are not limited to schizophrenia, schizoaffective disorder, attention deficit disorder, dysthymic disorder, major depressive disorder, mania, obsessive-compulsive disorder, psychoactive substance use disorders, anxiety, panic disorder, as well as bipolar affective disorder, e.g., severe bipolar affective (mood) disorder (BP-I), bipolar affective (mood) disorder with hypomania and major depression (BP-II). Further CNS-related disorders include, for example, those listed in the American Psychiatric Association's Diagnostic and Statistical manual of Mental Disorders (DSM), the most current version of which is incorporated herein by reference in its entirety.

As used herein, “differential expression” or differentially expressed” includes both quantative and qualitative differences in the temporal and/or cellular expression pattern of a gene, e.g., the programmed cell death genes disclosed herein, among, for example, normal cells and cells undergoing programmed cell death. Genes which are differentially expressed can be used as part of a prognostic or diagnostic marker for the evaluation of subjects at risk for developing a disorder characterized by deregulated programmed cell death. Depending on the expression level of the gene, the progression state of the disorder can also be evaluated.

### Arrays and Microarrays

The term “array” refers to a set of nucleic acid sequences disclosed herein. Preferred arrays contain numerous genes. The term can refer to all of the sequences disclosed herein but could also include sequences not disclosed, for example, sequences included as controls for specific biological processes. A “subarray” is also an array but is obtained by creating an array of less than all of the sequences in a starting array. For example, an array of programmed cell death cDNAs, such as those disclosed herein.

In one embodiment of the invention, an array comprising the nucleic acid sequences disclosed herein.

The array can include the maximum number of disclosed sequences or can be based on increments of sequences to form a subarray of the maximum number of sequences.

Thus, in one embodiment of the invention, the invention is directed to an array comprising the sequences disclosed (the maximum number of sequences) in increments of about 10, i.e., 10, 20, 30, 40, 50, 60, 70, 80, 90, 100, 110, etc. In another embodiment, the sequences are found in increments of about 50, i.e., 50, 100, 150, 200, 250, 300, etc., up to the maximum number in the array. In a further embodiment, the sequences are found in increments of about 100, i.e., 100, 200, 300, 400, etc., up to the maximum number of sequences. In one embodiment, each of these subarrays contains at least one novel gene. In one embodiment of the invention, there is the proviso that the novel gene is not rlrx015 f and h, rlrx018 a and b, rlrx020 a, b, c, d, e, f, and g (NARC1), and rlrx022 f and h (NARC2). In a preferred embodiment, the subarray of the complete array of nucleic acid sequences disclosed herein is in increments of about 100 sequences. In a more preferred embodiment, the subarray is in increments of about 500 sequences. In a still more preferred embodiment, the subarray is in increments of about 1000 sequences.

In another embodiment of the invention, the invention is directed to a subarray comprising the nucleic acid sequences disclosed herein. The same types of ranges accordingly applies to this subarray. Thus in one embodiment of the invention, the invention is directed to nucleic acids in this subarray in increments of about 10, i.e., 10, 20, 30, 40, 50, 60, 70, 80, 90, 100, 110, etc. up to the maximum number of sequences in the subarray. In another embodiment, the sequences are found in increments of about 50, i.e., 50, 100, 150, 200, 250, 300, 350, etc., up to the maximum number in the subarray. In a further embodiment, the sequences are found in increments of about 100, i.e., 100, 200, 300, 400, etc., up to the maximum number of sequences in the subarray.

The same types of ranges apply to subarrays, such as that described herein, and to functional subarrays, including but not limited to, those disclosed herein, including but not limited to, apoptosis, cell proliferation, cytoskeletal reorganization, secretion, synapse formation, hormone response, synaptic vesicle release, and calcium signal transduction. In one embodiment of the invention, the invention is directed to a function-biased array comprising sequences having a specific function in increments of about 10, i.e., 10, 20, 30, 40, 50, 60, 70, 80, 90, 100, 110, etc. In another embodiment, the sequences are found in increments of about 50, i.e., 50, 100, 150, 200, 250, 300, etc., up to the maximum number of such sequences in the subarray. In a further embodiment, the sequences are found in increments of about 100, i.e., 100, 200, 300, 400, etc., up to the maximum number of such sequences. In one embodiment, each of these subarrays contains at least one novel gene, as described herein. In one embodiment of the invention, there is the proviso that the novel gene is not rlrx015 f and h, rlrx018 a and b, rlrx020 a, b, c, d, e, f, and g (NARC1), and rlrx022 f and h (NARC2). In a preferred embodiment, the functional subarray is in increments of about 100 sequences. In a more preferred embodiment, the subarray is in increments of about 500 sequences. In a still more preferred embodiment, the subarray is in increments of about 1000 sequences.

These functional subarrays and incremental numbers of nucleic acid sequences in such functional subarrays can be derived from any of the sequences described herein, which includes both novel and known sequences, or can be derived exclusively from sequences disclosed herein and can comprise only the novel genes disclosed herein.

Accordingly, the invention encompasses subarrays derived from the brain-biased library comprising at least the incremental number of sequences, as described above or functional subarrays. As discussed, in one embodiment, one or more novel genes is comprised in the increment. Further, as discussed, in another embodiment the subarray is assembled with the proviso that the novel gene is not rlrx015 f and h, rlrx018 a and b, rlrx020 a, b, c, d, e, f, and g (NARC1), and rlrx022 f and h (NARC2).

Accordingly, the invention is further directed to a functional array as described above comprising at least the incremental numbers of sequences, as described above. In one embodiment, the subarray contains at least one novel gene as designated herein. In another embodiment, the array is assembled with the proviso that the novel gene is not rlrx015 f and h, rlrx018 a and b, rlrx020 a, b, c, d, e, f, and g (NARC1), and rlrx022 f and h (NARC2).

In one embodiment of the invention, the functional subarray comprises nucleic acid sequences expressed in programmed cell death as disclosed herein.

The array comprises not only the specific designated sequences but also variants of these sequences, as described herein. As described, variants include, allelic variants, homologs from other loci in the same animal, orthologs, and sequences sufficiently similar such that they fulfill the requisites for sequence similarity/homology as described herein.

Further, the array not only comprises the specific designated sequences, but also comprises fragments thereof. As described herein, the range of fragments will vary depending upon the specific sequence involved. Accordingly, the range of fragments is considerable, for example, 10, 15, 20, 25, 30, 35, 40, 45, 50, 55, 60, 65, 70, 75, 80, 85, 90, 95, 100, 125, 150, 200, 250, 300, 350, 400, 450, 500, 550, 600, 650, 700, 750, 800, 850, 900, 950, 1000 etc. In no way, however, is a fragment to be construed as having a sequence identical to that which may be found in the prior art.

The array can be used to assay expression of one or more genes in the array.

In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development and differentiation, tumor progression, progression of other diseases, in vitro processes, such as cellular transformation and senescence, autonomic neural and neurological processes, such as, for example, pain and appetite, and cognitive functions, such as learning or memory.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

In one embodiment, the array, and particularly subarrays containing one or more of the nucleic acid sequences related to programmed cell death, are useful for diagnosing disease or predisposition to disease involving apoptosis. These disorders include, but are not limited to, those discussed in detail herein. In addition, the array or subarrays created therefrom are useful for diagnosing active disorders of the central nervous system or for predicting the tenancy to develop such disorders. Disorders of the central nervous system include, but are not limited to, those disclosed in detail herein. Furthermore, the array and subarrays thereof are useful for diagnosing an active disorder or predicting the tendency to develop a disorder including, but not limited to, disorders involving secretion/synaptic vesicle release, cell proliferation, cytoskeletal reorganization, stress response/hormone response; and calcium signal transduction.

The array is also useful for ascertaining expression of one or more genes in model systems in vitro or in vivo. Various model systems have been developed to study normal and abnormal processes, including, but not limited to, apoptosis.

Apoptosis can be actively induced in animal cells by a diverse array of triggers that range from ionizing radiation to hypothermia to viral infections to immune reactions. Majno et al. (1995) Amer. J. Pathol. 146:3-15; Hockenberry et al. (1995) Bio Essays 17:631-638; Thompson et al. Science 267:1456-1462 (1995).

Transgenic mouse models have been developed for familial amyotrophic lateral sclerosis, familial Alzheimer's disease and Huntington's disease, reviewed in Price et al. (1998) Science 282:1079-1083. Amyotrophic lateral sclerosis is the most common adult onset motor neuron disease. Alzheimer's disease is the most common cause of dementia in adult life. It is associated with the damage of regions and neurocircuits critical for cognition and memory, including neurons in the neocortex, hippocampus, amygdala, basal forebrain cholinergic system, and brain stem monoaminergic nuclei. Neurological diseases that are associated with autosomal dominant trinucleotide repeat mutations include Huntington's disease, several spinal cerebellar ataxias and dentatorubral pallidoluysian atrophy. SCA-1 and SCA-3 or Machado-Joseph disease are characterized by ataxia and lack of coordination. In Huntington's disease, symptoms are related to degeneration of subsets of striatal and cortical neurons. Apoptosis is thought to play a role in the degeneration of these cells. In SCA-1, SCA-3, and in dentatorubral pallidoluysian atrophy, a variety of cell populations, and particularly cells in the cerebellum, have been shown to degenerate. See Price et al. above, which is incorporated by reference in its entirety for the teachings of model systems related to neurodegenerative diseases.

Mouse models have been developed for non-obese diabetic mice, to study disease progression for the treatment of autoimmune diabetes mellitus. Bellgrau et al. (1995) Nature 377:630-632. Models have also been developed in mice wherein the mice lack one or two copies of the p53 gene. Study of these mice has shown that apoptosis is involved in suppressing tumor development in vivo. Lozano et al. (1998) Semin. Canc. Biol. 8:337-344. Another animal model relevant to the study of apoptosis involves the targeted gene disruption of caspase genes creating caspase gene knockout mice. Colussi et al. (1999) J. Immun. Cell. Biol. 77:58-63. A further mouse model pertains to cold injury in mice, such injury inducing neuronal apoptosis. Murakami et al. (1999) Prog. Neurobiol. 57:289-299.

Knockout mice have been created for Apaf1. In these mice, defects are found in essentially all tissues whose development depends on cell death, including loss of interdigital webs, formation of the palate, control of neuron cell number, and development of the lens and retina. Cecconi et al. (1998) Cell 94:727-737.

Caspase knockout mice have also been achieved for caspase 1, 2, 3, and 9. Green (1998) Cell 94:695-698.

The array allows the simultaneous determination of a battery of genes involved in these processes and thus provides multiple candidates for in vivo verification and clinical testing. Because the array allows the determination of expression of multiple genes, it provides a powerful tool to ascertain coordinate gene expression, that is co-expression of two or more genes in a time and/or tissue-specific manner, both qualitatively and quantitatively. Thus, genes can be grouped on the basis of their expression per se and/or level of expression. This allows the classification of genes into functional categories even when the gene is completely uncharacterized with respect to function. Accordingly, if a first gene is expressed coordinately with a second gene whose function is known, a putative function can be assigned to that first gene. This first gene thus provides a new target for affecting that function in a diagnostic or therapeutic context. The larger the number of genes in an array, the greater is the probability that numerous known genes having the same or similar function will be expressed. In this case, the coordinate expression of one or more novel genes (with respect to function and/or structure) strongly allows discovery of genes in the same functional category as the known genes.

Accordingly, the array of the invention provides for “internal control” groups of genes whose functions are known and can thus be used to identify genes as being in the same functional category of the control group if they are coordinated expressed.

As an alternative to relying on such internal control groups, external control groups can be added to the array. The genes in such a group would have a known function. Genes coordinately expressed with these genes would thus be prima facie involved in the same function.

Therefore, the array provides a method not only for discovering novel genes having a specific function but also for assigning function to genes whose function is unknown or assigning to a known gene an additional function, previously unknown for that gene.

Accordingly, as disclosed and exemplified herein, previously characterized genes were grouped into new functional categories (i.e., previously the function was not known to be possessed by that gene). Furthermore, several uncharacterized genes could be functionally classified on the basis of coordinate expression with the “internal control group of genes”. In a specific embodiment, disclosed and exemplified herein, genes related to programmed cell death in brain were selected. The array could, accordingly be used to select for genes related to other important biological processes, such as those disclosed herein. Nucleic acid from any tissue in any biological process is hybridized to nucleic acid sequences in an array. The expression pattern of genes in the array allows for their classification into functional groups based on specific expression patterns. Internal or external control genes (i.e. genes known to be expressed in the specific tissue/biological process) provide verification to classify other genes in the specific category.

Thus, the array is also useful for discovering genes involved in a biological process. This is specifically disclosed in the Examples, in which a subarray of the sequences described herein was developed. The subarray is composed of genes related to programmed cell death, especially in brain. Some of the genes were previously known to function in programmed cell death. Others were known per se, but not known to function in programmed cell death. Still others had not previously been characterized at the level of structure or expression.

The invention is thus directed to subarrays constructed by screening the array against various functional control groups, such as secretion/synaptic vesicle release, cell proliferation, secretion/synaptic vesicle release/cytoskeletal reorganization, stress response/hormone response, calcium signal transduction, apoptosis, and cytoskeleton/synapse cytoskeleton, or alternatively constructed, as exemplified herein, by screening against RNA (cDNA) from a specific biological sample, such as a programmed cell death model.

The subarray can be further divided based on related function or other parameters. In the present case, the designated NARC genes are of particular interest in programmed cell death. Therefore, in one embodiment the invention is directed to one or more of these genes, useful as disclosed herein. In one embodiment, they are useful as a control group for assigning function to other genes. Individually, they are subject to any of the various uses discussed herein.

Just as the array was useful for identifying programmed cell death genes, other relevant normal biological models include differentiation programs and disorders such as those disclosed herein.

The array is also useful for drug discovery. Candidate compounds can be used to screen cells and tissues in any of the biological contexts disclosed herein, such as pathology, development, differentiation, etc. Thus the expression of one or more genes in the array can be monitored by using the array to screen for RNA expression in a cell or tissue exposed to a candidate compound. Compounds can be selected on the basis of the overall effect on gene expression, not necessarily on the basis of its effect on a single gene. Thus, for example, where a compound is desired that affects a particular first gene or genes but has no effect on a second gene or genes, the array provides a way to globally monitor the effect on gene expression of a compound.

Alternatively, it may be desirable to target more than one gene, i.e. to modulate the expression of more than one gene. The array provides a way to discover compounds that will modulate a set of genes. All genes of the set can be upregulated or down-regulated. Alternatively, some of the genes may be upregulated and others downregulated by the same compound. Moreover, compounds are discoverable that modulate desired genes to desired degrees.

In the context of drug discovery, functional subarrays of genes are especially useful. Thus, using the methods disclosed herein and those routinely available, groups of genes can be assembled based on their relationships to a specific biological function. The expression of this group of genes can be used for diagnostic purposes and to discover compounds relevant to the biological function. Thus, the subarray can provide the basis for discovering drugs relevant to treatment and diagnosis of disease, for example those disclosed herein.

In the present case, the group of genes whose expression is correlated with programmed cell death can be used to discover compounds that affect programmed cell death, and especially disorders in which programmed cell death is involved. These include but are not limited to those disclosed herein.

Apoptosis can be triggered by the addition of apoptosis-promoting ligands to a cell in culture or in vivo. In one embodiment of the invention, therefore, the arrays and subarrays described herein are useful to identify genes that respond to apoptosis-promoting ligands and conversely to identify ligands that act on genes involved in apoptosis. Apoptosis can also be triggered by decreasing or removing an apoptosis-inhibiting or survival-promoting ligand. Accordingly, apoptosis is triggered in view of the fact that the cell lacks a signal from a cell surface survival factor receptor. Ligands include, but are not limited to, FasL. Death-inhibiting ligands include, but are not limited to, IL-2. See Hetts et al. (1998) JAMA 279:300-307 (incorporated by reference in its entirety for teaching of ligands involved in active and passive apoptosis pathways.) Central in the pathway, and also serving as potential molecules for inducing (or releasing from inhibition) apoptosis pathways include FADD, caspases, human CED4 homolog (also called apoptotic protease activating factor 1), the Bcl-2 family of genes including, but not limited to, apoptosis promoting (for example, Bax and Bad) and apoptosis inhibiting (for example, Bc1-2 and Bc1-xl) molecules. See Hetts et al., above.

Multiple caspases upstream of caspase-3 can be inhibited by viral proteins such as cowpox, CrmA, and baculovirus, p35, synthetic tripeptides and tetrapeptides inhibit casepase-3 specifically (Hetts, above). Accordingly, the arrays and subarrays are useful for determining the modulation of gene expression in response to these agents.

The array is also useful for obtaining a set of human (or other animal) orthologs that can be used for drug discovery, treatment, diagnosis, and the other uses disclosed herein. The subarrays can be used to specifically create a corresponding human (or other animal) subarray that is relevant to a specific biological function. Accordingly, a method is provided for obtaining sets of genes from other organisms, which sets are correlated with, for example, disease or developmental disorders.

In a preferred embodiment of the invention, the arrays and subarrays disclosed herein are in a “microarray”. The term “microarray” is intended to designate an array of nucleic acid sequences on a chip. This includes in situ synthesis of desired nucleic acid sequences directly on the chip material, or affixing previously chemically synthesized nucleic acid sequences or nucleic acid sequences produced by recombinant DNA methodology onto the chip material. In the case of recombinant DNA methodology, nucleic acids can include whole vectors containing desired inserts, such as phages and plasmids, the desired inserts removed from the vector as by, PCR cloning, cDNA synthesized from mRNA, mRNA modified to avoid degradation, and the like.

A series of state-of-the-art reviews of the technology for production of nucleic acid microarrays in various formats and examples of their utilization to address biological problems is provided in Nature Genetics, 21 Supplement, January 1999. These topics include molecular interactions on microarrays, expression profiling using cDNA microarrays, making and reading microarrays, high density synthetic oligonucleotide arrays, sequencing and mutation analysis using oligonucleotide microarrays, the use of microarrays in drug discovery and development, gene expression informatics, and use of arrays in population genetics. Various microarray substrates, methods for processing the substrates to affix the nucleic acids onto the substrates, processes for hybridization of the nucleic acid on the substrate to an external nucleic acid sample, methods for detection, and methods for analyzing expression data using specific algorithms have been widely disclosed in the art. References disclosing various microarray technologies are listed below.

Lashkari et al. (1997) “Yeast Microarrays for Genome Wide Parallel Genetic and Gene Expression Analysis”, Proc. Natl. Acad. Sci. 94:13057-13062; Ramsay (1998) “DNA Chips: State-of-the-Art”, Nature Biotechnology 16:40-44; Marshall et al. (1998) “DNA Chips: An Array of Possibilities”, Nature Biotechnology 16:27-31; Wodicka et al. (1997) “Genome-Wide Expression Monitoring In Saccharomyces Cerevisiae”, Nature Biotechnology 15:1359-1367; Southern et al. (1999) “Molecular Interactions On Microarrays”, Nature Genetics 21(1):5-9; Duggan, et al. (1999) Nature Genetics 21(1):10-14; Cheung et al. (1999) “Making and Reading Microarrays”, Nature Genetics 21(1):15-19; Lipshutz et al. (1999) “High Density Synthetic Oligonucleotide Arrays”, Nature Genetics 21(1):20-24; Bowtell (1999) Nature Genetics 21:25-32; Brown et al. (1999) “Exploring the New World of the Genome with DNA Microarrays” Nature Genetics 21(1):33-37; Cole et al. (1999) “The Genetics of Cancer—A 3D Model” Nature Genetics 21(1):38-41; Hacia (1999) “Resequencing and Mutational Analysis Using Oligonucleotide Microarrays”, Nature Genetics 21(1):42-47; Debouck et al. (1999) “DNA Microarrays in Drug Discovery and Development”, Nature Genetics 21(1):48-50; Bassett, Jr. et al. (1999) “Gene Expression Informatics—It's All In Your Mine”, Nature Genetics 21(1):51-55; Chakravarti (1999) “Population Genetic—Making Sense Out of Sequence”, Nature Genetics 21(1):56-60; Chee et al. (1996) “Accessing Genetic Information with High-Density DNA Arrays”, Science 274:610-614; Lockhart et al. (1996) “Expression Monitoring by Hybridization to High-Density Oligonucleotide Arrays”, Nature Biotechnology 14:1675-1680; Tamayo et al. (1999) “Interpreting Patterns of Gene Expression with Self-Organizing Maps: Methods and Application to Hematopoietic Differentiation”, Proc. Natl. Acad. Sci. 96:2907-2912; Eisen et al. (1998) “Cluster Analysis and Display of Genome-Wide Expression Patterns”, Proc. Natl. Acad. Sci. 95:14863-14868; Wen et al. (1998) “Large-Scale Temporal Gene Expression Mapping of Central Nervous System Development”, Proc. Natl. Acad. Sci. 95:334-339; Ermolaeva et al. (1998) “Data Management and Analysis for Gene Expression Arrays”, Nature Genetics 20:19-23; Wang et al. (1998) “A Strategy for Genome-Wide Gene Analysis: Integrated Procedure for Gene Identification”, Proc. Natl. Acad. Sci. 95:11909-11914; U.S. Pat. No. 5,837,832; U.S. Pat. No. 5,861,242; WO 97/10363.

In the instant case, the microarray contains nucleic acid sequences on a Biodyne B filter. However, any medium, including those that are well-known and available to the person of ordinary skill in the art, to which nucleic acids can be affixed in a manner suitable to allow hybridization, are encompassed by the invention. This includes, but is not limited to, any of the membranes disclosed in the references above, which are incorporated herein for reference to those membranes, and other membranes that are commercially available, including but not limited to, nitrocellulose-1, supported nitrocellulose-1, and Biodyne A, which is a neutrally-charged nylon membrane suitable for Southern transfer and dot blotting procedures. (All are available from Life Technologies.)

### Summary

Programmed cell death (PCD) in rat cerebellar granule neurons (CGNs) induced by potassium (K+) withdrawal has been shown to depend on de novo RNA synthesis. The inventors characterized this transcriptional component of CGN programmed cell death using a custom-built brain-biased cDNA array representing over 7000 different rat genes. Consistent with carefully orchestrated mRNA regulation, the profiles of 234 differentially expressed genes segregated into distinct temporal groups (immediate early, early, middle, and late) encompassing genes involved in distinct physiological responses including cell-cell signaling, nuclear reorganization, apoptosis, and differentiation. A set of 64 genes, including 22 novel genes, were regulated by both K+ withdrawal and kainate treatment. Thus, by using array technology, they were able to broadly characterize physiological responses at the transcriptional level and identify novel genes induced by multiple models of programmed cell death.

### Background

In neurons, programmed cell death is an essential component of neuronal development (Jacobson et al. 1997; Pettmann and Henderson (1998); Pettmann and Henderson (1998) Neuron 20:633-747) and has been associated with many forms of neurodegeneration (Hetts (1998) Journal of the American Medical Association 279:300-307). In the cerebellum, granule cell development occurs postnatally. The final number of neurons represents the combined effects of additive processes such as cell division and subtractive processes such as target-related programmed cell death. Depolarization due to high concentrations (25 mM) of extracellular potassium (K+) promotes the survival of cerebellar granule neurons (CGNs) in vitro. CGNs maintained in serum containing medium with high K+ will undergo programmed cell death when switched to serum-free medium with low K+ (5 mM) (D'Mello et al. (1993) Proc. Natl. Acad. Sci. USA 90:10989-10993; Miller and Johnson (1996) Journal of Neueroscience 16:7487-7495). The resulting programmed cell death has a transcriptional component that can be blocked by inhibitors of new RNA synthesis (Galli et al. (1995) Journal of Neuroscience 15:1172-1179; and Schulz and Klockgether (1996) Journal of Neuroscience 16:4696-4706). Traditionally, the regulation of limited numbers of specific genes were characterized during CGN programmed cell death using Northern nucleic acid hybridization (e.g. PTZ-17, Roschier et al. (1998) Biochemical and Biophysical Research Communications 252:10-13), reverse transcription polymerase chain reaction (RT-PCR; e.g. c-jun, cyclophilin, cyclin D1, c-fos and caspase (Miller et al. (1997) Journal of Cell Biology 139:205-217), and in situ hybridization (e.g. RP-8; Owens et al. (1995) Developmental Brain Research 86:35-47).

High-density cDNA arrays have been successfully used to characterize genome-wide mRNA expression in yeast (Lashkari et al. (1997) Proc. Natl. Acad. Sci. USA 94:13057-13062; Wodicka et al. (1997) Nature Biotechnology 15:1997). In higher eukaryotes, the strategy has been to array as many sequences as possible from known genes, from expressed sequence tags (ESTs), or from uncharacterized cDNA clones from a library (Bowtell (1999) Nature Genetics 21:25-32; Duggan et al. (1999) Nature Genetics 21:10-14; Marshall and Hodgson (1998) Nature Biotechnology 16:27-31; and Ramsay (1998) Nature Biotechnology 16:40-44). Global RNA regulation during cellular processes including cell-cycle regulation (Cho et al. (1998) Molecular Cell 2:65-73, and

Spellman et al. (1998) Mol. Biol. Cell. 95:14863-14868), fibroblast growth control (Iyer et al. (1999) Science 283:83-87), metabolic responses to growth medium (Derisi and Brown (1997) Science 278: 680-686), and germ cell development (Chu et al. (1998) Science 282:699-705) have been temporally monitored using arrays. The program of gene expression delineated in these studies demonstrated a correlation between common function and coordinate expression, and also provided a comprehensive, dynamic picture of the processes involved (Brown and Botstein (1999) Nature Genetics 21:33-37). For the cellular process of programmed cell death, a DNA chip has been used to identify twelve known genes as differentially expressed between two conditions, etoposide-treated and untreated cells (Wang et al. (1999) FEBS Letters 445:269-273).

A genome-wide approach for the comprehensive characterization of the transcriptional component of rat CGN programmed cell death and for identification of novel neuronal apoptosis genes requires an array consisting of both known and novel rat cDNAs. The inventors constructed a brain-biased and programmed cell death-enriched clone set by arraying ˜7300 consolidated ESTs from two cDNA libraries cloned from rat frontal cortex and differentiated PC12 cells deprived of nerve growth factor (NGF), and >300 genes that are known markers for the central nervous system and/or programmed cell death. They reproducibly and simultaneously monitored the expression of the genes at 1, 3, 6, 12, and 24 hours after K+ withdrawal. They then categorized the regulated genes by time course expression pattern to identify cellular processes mobilized by CGN programmed cell death at the RNA level. In particular they focused on the expression profiles of many known pro- and anti-apoptotic regulatory proteins, including transcription factors, Bcl-2 family members, caspases, cyclins, heat shock proteins (HSPs), inhibitors of apoptosis (IAPB), growth factors and receptors, other signal transduction molecules, p53, superoxide dismutases (SODs), and other stress response genes. Finally, they compared the time courses of regulated genes induced by K+ withdrawal in the presence or absence of serum to those induced by glutamate toxicity. Thus, they identified a restricted set of relevant genes regulated by multiple models of programmed cell death in CGNs.

### Results

Construction and Validation of a Brain-biased cDNA Microarray

In order to characterize the transcriptional component of neuronal apoptosis in rat cerebellar granule neurons, the inventors constructed a cDNA array, called Smart Chip™ I, that contains primarily rat brain genes. Two cDNA libraries were cloned from rat frontal cortex and nerve growth factor-deprived rat PC12 cells to enrich for cDNAs expressed in the central nervous system and in one in vitro model of neuronal apoptosis. Expressed sequence tags (ESTs) from the 5′-end were identified for 8,304 clones in the cortical library and 5,680 in the PC12 library. These 13,984 ESTs were condensed into 7,399 unique sequence clusters by using the Basic Local Alignment Search Tool (BLAST) sequence comparison analysis (Altschul et al. 1990) to identify ESTs with overlapping sequence. One representative clone was chosen from each of 7,296 of the unique sequence clusters and prepared for PCR amplification using a robotic sample processor. In addition to the ESTs, PCR templates were prepared for 289 known DNA sequences, including negative controls, genes with known function in the CNS and/or during programmed cell death, and genes previously identified as regulated by CGN programmed cell death using differential display (data not shown). To check the fidelity of the set of array elements, a robotic sample processor was used to randomly choose 212 clones for sequencing. Ten clones produced poor sequence. The remaining 202 matched their seed sequence (data not shown), implicating 100% fidelity in sample tracking

A sample volume of 20 nl from each of the 7584 PCR products was arrayed onto nylon filters at a density of ˜64/cm2 using a pin robot. The arrayed DNA elements were denatured and covalently attached to the nylon filters for use in reverse Northern nucleic acid hybridization experiments. In a typical experiment, “radiolabeled RNA”, 1 μg polyA RNA radiolabeled by 33P-dCTP incorporation during cDNA synthesis, was hybridized to triplicate arrays following RNA hydrolysis. Subsequently, the filters were washed and exposed to phosphoimage screens. Gene expression was quantified for each array element by digitizing the phosphoimage-captured hybridization signal intensity. An illustration that the coefficient of variation between triplicate hybridizations averaged less than 0.2 for genes whose intensities were above a threshold of 30-40 units is described herein. From control experiments when in vitro transcribed RNAs were deliberately spiked into samples, this threshold amounted to a copy number of less than 1 in 100,000 (data not shown).

### Tissue Distribution of Brain-biased Smart Chip ESTs

To characterize the brain-biased cDNA array and possibly identify brain-specific genes, radiolabeled RNA from ten different normal rat tissues was hybridized to Smart Chip. Compared to heart, kidney, liver, lung, pancreas, skeletal muscle, smooth muscle, spleen, and testes, radiolabeled rat brain RNA produced more hybridization signal intensity against most of the brain-biased array elements. After data normalization and averaging between replicates, the threshold of detection was determined for each experiment and the number of genes detected for each tissue was tabulated. Most (6127 out of 7296) but not all of the ESTs were detected in at least one of the tissues profiled. The number of genes detected in brain was the highest. 582 genes appeared to be brain-specific, as defined by detection above threshold for brain but below threshold for any of the other nine tissues.

### The Physiology of CGN KCl/serum-withdrawal as Characterized by Transcription Profiling on Smart Chip

Using the brain-biased, programmed cell death nucleic acid-enriched Smart Chip, global mRNA expression was profiled throughout a time course of KCl/serum-withdrawal-induced cell death in primary cultures of CGNs. The transcription-dependent CGN programmed cell death was coordinated, resulting in less than 30% survival at 24 hours post-withdrawal as quantified by cell counting (data not shown). RNA samples, designated “treated”, were isolated at 1, 3, 6, 12, and 24 hours after switching post-natal day eight CGNs from medium containing 5% serum and 25 mM KCl to serum-free medium with 5 mM KCl. For controls, the 5% serum/25 mM KCl medium was replaced, and “sham” RNA at 1, 3, 6, 12, and 24 hours was isolated.

Since the average coefficient of variation for gene expression intensities between triplicate hybridizations was less than 0.2, genes regulated at least three-fold during the time course (790 out of 6818 detected; data not shown) were further addressed. Using hierarchical clustering algorithms (see Experimental Procedures), the regulated genes were ordered based on their gene expression pattern across the ten experimental points (five time points, sham and treated). The hierarchy of relatedness between gene expression profiles are disclosed. The first major branch point segregated those genes regulated by sham treatment (first five columns), and those regulated by KCl/serum-withdrawal treatment only (last five columns). A majority of genes (556) were regulated by sham treatment. These genes included trk A, PSD-95, SV 2A, and VAMP 1, and were most likely induced by serum-add-back in the sham since the medium was exchanged at t=0 with unconditioned medium.

The expression pattern of 234 programmed cell death-induced genes that were regulated by KCl/serum-withdrawal only, and were not regulated by serum-add-back in the sham experiments ar described herein. Their coefficient of variation in expression level throughout the five serum-add-back experiments was less than 20%. Since the serum-add-back experiments were non-discriminating for these genes, the serum-add-back data were averaged to generate a single control data set for clustering with the KCl/serum withdrawal time course. Four apparent temporal regulation classes were designated immediate early (peaking at 1 hour followed by rapid decay), early (peaking at 3-6 hours), middle (peaking at 6-12 hours), and late (up-regulated at 24 hours). Almost all of the immediate early genes encoded proteins with known roles in regulating secretion and synaptic vesicle release including synaptotagmin, synaphin, NSG-1, calcium calmodulin-dependent kinase II, synapsin, complexin, LDL receptor, and fodrin. Histones 1, 2A, and 3 fell in the early class. Middle genes comprised several known genes induced by programmed cell death or stress, including caspase 3, the mammalian oxy R homolog, cytochrome c oxidase and protein phosphatase Wip-1. Functions encoded for by late genes could be effectors of survival mechanisms including inhibitory neurotransmission (GAD, GABA-A receptor, GABA transporter), cell adhesion (nexin, basement membrane protein 40, phosphacan, rat GRASP), down-regulation of excitatory neurotransmission (glutamate transporter, sodium-dependent glutamate/aspartate transporter), leukotriene metabolism (dithio)ethione-induced NADP-dependent leukotriene B4 12-hydroxydegydrogenase, leukotriene A-4 hydrolase), protein stabilization (cysteine proteinase inhibitor cystatin C, N-alpha-acetyl transferase, CaBP2, elongation factor 1-gamma, APG-1), and ionic balance and cell volume (SLC12A integral membrane protein transporter). Based on four distinct waves of gene expression, the major transcriptional reponses observed for KCl/serum-withdrawal included initial up-regulation of synaptic vesicle release/recycling, then, of histone biosynthesis, followed by various constituents of programmed cell death regulation and stress-response signaling, and finally, of multiple survival mechanisms. The apparent changes in transcription most likely also reflect changes in the relative cell populations, since late mRNAs may be markers of neurons and non-neuronal cells which have survived KCl/serum-withdrawal at 24 hours. Another contributing factor may be the presence of two populations of dying neurons that respond with different kinetics to serum versus KCl withdrawal, as has been described by other groups.

### Neuronal Apoptosis Regulated Candidates (NARCs) Regulated by Multiple Models of Programmed Cell Death

112 novel ESTs were significantly regulated by KCl/serum-withdrawal in rat CGNs (data not shown). Some exhibited similar expression profiles throughout KCl/serum-withdrawal and serum-add-back to genes with known function during programmed cell death, such as caspase 3. The temporally-coupled expression of these novel genes may reflect related functionality with caspase 3, since they probably share common RNA regulatory elements, including those regulating initiation, elongation, processing, and/or stability. Apparent coordinate transcriptional up-regulation of synaptic vesicle release/recycling possibly reflects a physiological response to near cessation of synaptic transmission that may or may not contribute to the programmed cell death pathway. To help further distinguish genes that are specifically regulated in response to programmed cell death, CGN programmed cell death induced by glutamate (excitatory neurotransmitter) toxicity was studied. In addition, the effect of KCl-withdrawal alone on gene expression was examined. This was done under defined medium conditions to minimize the effect of serum on the sham and treated samples.

Rat CGNs from post-natal day seven pups were isolated as before and plated into basal medium Eagle containing “high”, 10% dialysed fetal bovine serum, and “high”, 25 mM KCl. After two days in culture, the medium was replaced with neurobasal medium supplemented with “low”, 0.5% serum, and high KCl. To initiate KCl-withdrawal on day eight, the KCl concentration was switched to 5 mM for the treated samples. The same low serum, high KCl, neurobasal medium was replaced in the controls to minimize gene induction by high serum. For the glutamate toxicity experiment, the cells were treated for 30 min in sodium-free Locke's medium with or without 100 μM kainate for treated samples and controls, respectively.

After isolation from treated and control samples at 1, 3, 6, and 12 hours after KCl-withdrawal and 2, 4, 6, 12 hours after kainate treatment, mRNA was subjected to expression profiling analysis on Smart Chip I. An illustration of the changes in gene expression that occur over time when CGNs are induced to undergo programmed cell death by KCl/serum-withdrawal, KCl-withdrawal alone, or kainate treatment is disclosed. In the scatter plots, due to differential expression, large numbers of regulated genes migrated away from a line of slope one when withdrawn (W) or treated (T) samples were compared to control (C). The sham treated cells for the KCl/serum-withdrawal clearly responded to basal medium serum-add-back, whereas shams for KCl-withdrawal alone and kainate treatment did not respond to conditioned neurobasal medium add-back. Profiling across the mRNA levels of thousands of genes provided a clear index of changes in overall cell physiology.

In general, apparent changes in gene expression were less robust in the cells cultured on neurobasal medium. The number of genes detected above threshold was similar for all three paradigms, 6634, 7017, and 6818, respectively, for KCl-withdrawal, kainate treatment, and KCl/serum withdrawal (data not shown). Yet the number of genes regulated by at least three-fold during KCl-withdrawal and kainate treatment was only 156 and 167, respectively (data not shown), compared to the 790 discussed above for KCl/serum withdrawal.

A hierarchical clustering algorithm was used to order the regulated genes based on their gene expression pattern across all CGN programmed cell death paradigms investigated. Twenty-six individual profiling experiments in duplicate or triplicate were performed across the 7584 rat genes on Smart Chip I using mRNA isolated from 5 serum-add-back time points, 5 KCl/serum-withdrawal time points, 4 time points each for sham and KCl-withdrawal, and 4 time points each for sham and kainate treatment.

The expression clusters generated by one hierarchical clustering algorithm are described herein. The inset shows a specific group of genes having similar expression patterns. This group includes genes known to be regulated in programmed cell death, for example caspase 3 and Wip 1, as well as other nucleic acid sequences on the array not previously known to be regulated. Those sequences meeting specific criteria were designated “neuronal apoptosis regulated candidate” (NARC). Criteria for designating such genes were based on specific expression criteria. Nucleic acid sequences having an expression pattern similar to genes known to be involved in apoptosis were designated as NARC sequences. The sequences of the rat neuronal apoptosis regulated candidates NARC SC 1 (SEQ ID NO:1), NARC 10A (SEQ ID NO:4), NARC 1 (SEQ ID NO:5), NARC 12 (SEQ ID NO:6), NARC 13 (SEQ ID NO:7), NARC17 (SEQ ID NO:8), NARC 25 (SEQ ID NO:9), NARC 3 (SEQ ID NO:10), NARC 4 (SEQ ID NO:11), NARC 7 (SEQ ID NO:12 and 13), NARC 8 (SEQ ID NO:14), NARC 11 (SEQ ID NO:18 and 19), NARC 14A (SEQ ID NO:20), NARC 15 (SEQ ID NO:21), NARC 16 (SEQ ID NO:22), NARC 19 (SEQ ID NO:23), NARC 20 (SEQ ID NO:24), NARC 26 (SEQ ID NO:25), NARC 27 (SEQ ID NO:26), NARC 28 (SEQ ID NO:27), NARC 30 (SEQ ID NO:28), NARC 5 (SEQ ID NO:29), NARC 6 (SEQ ID NO:30), and NARC 9 (SEQ ID NO:31); and the human neuronal apoptosis regulated candidate homologs NARC 10C (SEQ ID NO:2), NARC 8B (SEQ ID NO:3), NARC 9 (SEQ ID NO:15), NARC2A (SEQ ID NO:16), NARC 16B (SEQ ID NO:17), NARC 1C (SEQ ID NO:32), NARC 1A (SEQ ID NO:33), and NARC 25 (SEQ ID NO:34) are set forth in the Sequence Listing.

### Gene Expression Validation by RT-PCR

Although the reproducibility in transcription profiling experiments was quite high (average CV<0.2), the gene expression regulation of known and novel genes was validated by semi-quantitative RT-PCR. The rat CGN model system was used to independently validate the expression of several NARC genes that had shown expression (when hybridized with sequences on the chip) related to programmed cell death. Reverse transcriptase-assisted PCR was performed to assess expression of NARC 1-7, 9, 12, 13, 15, and 16. Experimental samples received KCl withdrawal treatment. Control samples show cells receiving no treatment. The PCR reactions contained 10, 5, 2.5, 1.3, and 0.7 ng of total RNA each. The RT-PCR protocol is disclosed in the exemplary material herein. NARC 1, 2, 4, 5, 7, 9, 12, 13, 15, and 16 all showed significant increases in expression levels within 3-6 hours following KCl withdrawl.

### NARC1 and NARC2 Regulation In Vivo During Cerebellar Development

Two novel neuronal apoptosis regulated candidates, NARC1 and NARC2, were validated by in situ hybridization and shown to be coordinately up-regulated with caspase 3 during postnatal development when increased apoptosis is associated with synapse consolidation in the cerebellum (not shown).

### Experimental Procedures

### BLAST Sequence Comparison Analysis

ESTs determined for the 5′-end of cDNA clones picked from two cDNA libraries, rat frontal cortex (8,304 clones) and NGF-deprived differentiated PC12 cells (5,680 clones), ranged from 100-1000 nt in sequence length and averaged 500 nt (data not shown). Sequence comparisons were done using BLAST (Altschul et al. 1990). Contiguous matches defined a sequence cluster. Large clusters were checked by hand to eliminate apparent chimeras. From 13,984 sequences inputted, the analysis identified 5,779 singletons and 1,620 larger clusters (data not shown). The 5′-most clone was selected from the larger clusters. Because two 96-well microtiter plates of clones were missing, a total of 7,296 out of the 7,399 identified were selected for Smart Chip™ I.

cDNA Microarray Construction

Using a Genesis RSP 150 robotic sample processor (Tecan AG, Switzerland), bacterial cultures of individual EST clones from the two libraries were consolidated from 13,792 clones spanning 144 96-well microtiter plates to 7296 Smart Chip I clones spanning 76 plates. To prepare templates for array elements, oligonucleotide primers specific for vector sequences up- and downstream of the cloning site were used to amplify the cDNA insert by PCR. Following ethanol precipitation and concentration (to 1-10 mg/ml), the array element templates were resuspended in 3×SSC (1×SSC: 150 mM sodium chloride, 15 mM sodium citrate, pH 7.0). A sample volume of 20 nl from each template was arrayed onto nylon filters (Biodyne B, Gibco BRL Life Technologies, Gaithersburg, Md.) at a density of ˜64/cm2 using a 96-well format pin robot (THOR). After the filters were dry, the arrayed DNA was denatured in 0.4 M sodium hydroxide, neutralized in 0.1 M Tris-HCl, pH 7.5, rinsed in 2×SSC, and dried to completion.

### Array Hybridization

Rat poly A+ RNA was purchased from Clontech (Palo Alto, Calif.) for the organ recital or was isolated as total RNA from cultured CGNs using RNA STAT-60™ (Tel-Test, Inc., Friendswood, Tex.) and then prepared using Oligotex™ (Qiagen, Inc., Chatsworth, Calif.). Re-annealed 1 μg mRNA and 1 μg oligo(dT)30 was incubated at 50° C. for 30 min with SuperScript™ II as recommended by Gibco in the presence of 0.5 mM each deoxynucleotide dATP, dGTP, and dTTP, and 100μCi α33P-dCTP (2000-4000 Ci/mmol; NEN™ Life Science Products, Boston, Mass.). After purification over Chroma Spin™+TE-30 columns (Clontech), the labeled cDNA was annealed with 10 μg poly(dA)>200 and 10 μg rat Cot-1 DNA (prepared as described in Britten et al. (1974) Methods in Enzymology 29:263-418). At 2×106 cpm/ml, the annealed cDNA mixture was added to array filters in pre-annealing solution containing 100 mg/ml sheared salmon sperm DNA in 7% SDS (sodium dodecyl sulfate), 0.25 M sodium phosphate, 1 mM ethylenediaminetetraacetic acid, and 10% formamide. Following over night hybridization at 65° C. in a rotisserie-style incubator (Robbins Scientific, Sunnyvale, Calif.), the array filters were washed twice for 15 min at 22° C. in 2×SSC, 1% SDS, twice for 30 min at 65° C. in 0.2×SSC, 0.5% SDS, and twice for 15 min at 22° C. in 2×SSC. The array filters were then dried and exposed to phosphoimage screens for 48 h. The radioactive hybridization signals were captured with a Fuji BAS 2500 phosphoimager and quantified using Array Vision™ software (Imaging Research Inc., Canada). Array hybridizations for the organ recital, the CGN KCl only-withdrawal, and the CGN kainate treatment experiments were performed in triplicate; for the CGN KCl/serum-withdrawal, they were performed in duplicate.

### Transcription Profiling Data Analysis

For replicate array hybridizations, the distribution of signal intensities across all rat genes was normalized to a median of 100. Replicate measurements were averaged and a coefficient of variation (CV; standard deviation/mean for triplicates or the absolute value of the difference/mean for duplicates) was determined for each gene. The detection threshold was chosen for each hybridization experiment by graphing the moving average (with a window of 200) for CV versus mean gene expression intensity. The threshold was defined as the intensity at which lower intensities exhibited an average CV that was greater than 0.3. For most experiments, this threshold ranged from 10 to 40, and the number of genes detected above threshold ranged from 70% to 95%.

### CGN Cell Culture

CGNs were prepared from seven day old rat pups as previously described (Johnson and Miller (1996) Journal of Neuroscience 16:74877-7495). Briefly, cerebella were isolated, and meningeal layers and blood vessels were removed under a dissecting scope. Dissociated cells were plated at a density of 2.3×10⁵ cells/cm2 in basal medium Eagle (BME; Gibco) supplemented with 25 mM KCl, 10% dialyzed fetal bovine serum (Summit Biotechnology lot #04D35, Ft. Collins, Colo.), 100 U/ml penicillin, and 100 μg/ml streptomycin. Aphidicolin (Sigma, St. Louis, Mo.) was added to the cultures at 3.3 μg/ml, 24 hours after initial plating to reduce the number of non-neuronal cells to less than 1-5%.

For KCl/serum-withdrawal experiments, after seven days in culture, the treated cells were switched to 5 mM KCl, BME, no serum, while the shams received a medium replacement. By 24 hours post-withdrawal, less than 30% of the cells were surviving as assayed by Hoechts cell counts (data not shown). This apparent cell death could be rescued by actinomycin D at 2 μg/ml (data not shown).

For the KCl-withdrawal alone and kainate treatment experiments, on day two in culture, the medium was replaced with neurobasal medium (Gibco) supplemented with 25 mM KCl, 0.5% dialyzed fetal bovine serum, B27 supplement (Gibco), 0.5 mM L-glutamine (Gibco), 0.1 mg/ml AlbuMAX I (Gibco), 100 U/ml penicillin, 100 μg/ml streptomycin, and 3.3 μg/ml aphidicolin. On day seven, KCl-withdrawal was initiated by replacing the medium with 5 mM KCl while the shams received 25 mM. By 24 hours post-withdrawal, 40% of the cells were surviving as assayed by Hoechts cell counts (data not shown). As previously described, glutamate toxicity was induced by replacing the medium for 30 min with 5 mM KCl, 100 μM kainic acid (Sigma) in sodium free Locke's buffer, while the shams received no kainic acid (Coyle et al. (1996) Neuroscience 74:675-683). After 30 min, the supplemented neurobasal medium was replaced. By 12 hours post-withdrawal, 30% of the cells were surviving as assayed by Hoechts cell counts (data not shown). The KCl-withdrawal induced cell death was rescued by actinomycin D, whereas the kainate-induced was not.

### Expression Data Clustering Algorithms

After normalization and averaging of the KCl/serum-withdrawal data, 790 genes passed the following criteria over the 10 time points (5 treated, 5 sham) for input into heirarchical clustering analysis: 1. detection, maximum intensity greater than 30; 2. noise filter, the difference between maximum and minimum intensity greater than 30; and 3. regulation, fold induction between maximum and minimum intensity of at least 3 (data not shown). Hierarchical clusters were ordered based on Euclidian distances. 234 out of 790 genes that passed the significance filter described above were not regulated in the controls based on CV less than 0.2 for all five control time points (data not shown).

### RT-PCR

Oligonucleotide primer sequences specific for each EST validated by RT-PCR were selected from quality sequence regions and designed to obtain a melting temperature of 55-60° C. as predicted by PrimerSelect software (DNASTAR, Inc., Madison, Wis.) based on DNA stability measurements by (Breslauer et al. (1986) Proc. Natl. Acad. Sci. USA 83:3746-3750). The Stratagene Opti-Prime™ Kit (La Jolla, Calif.) was used to determine optimal RT-PCR amplification conditions for each primer pair. RT-PCR reactions on 2-fold serially diluted CGN programmed cell death cDNA were set up using the Genesis RSP 150 robotic sample processor and incorporating the optimal buffer conditions for each primer pair. Every robot run included primers specific for housekeeping genes to control for day to day differences in cDNA template dilutions. The number of cycles was adjusted to obtain a linear range of amplification by comparing the amount of product made from the serially diluted templates as assessed by agarose gel electrophoresis.

### Preparation of Array on Nylon

Procedure for Generating Labeled First Strand cDNA Using Superscript II Reverse Transcriptase

10 mL (100 mCi) 33P α-dCTP was dried down by SpeedVac.
In a separate tube, the following components were mixed:

### 1.0 ug Poly A+ RNA or 10 ug Total RNA

1 uL 1 ug/uL oligo-dT(30)
x uL DEPC-H₂O, to 10 uL
   The above sample was heated at 70° C. for 4 minutes and then placed on ice.

3. 8 uL from the oligo/RNA mixture (#2) was removed and used to resuspend the dried 3P3. The following components were added to the reaction:
4 uL 5× First Strand Buffer (comes with Superscript II RT)
2 uL 100 mM DTT
1 uL 10 mM dAGT-TPs
1 uL 0.1 mM cold dCTP
1 uL Rnase Inhibitor
1 uL Superscript II RT
   The reaction was incubated for 30 minutes at 50° C.

4. After incubation, 2 uL 0.5 M NaOH, and 2 uL 10 mM EDTA were added. The reaction was heated at 65° C., for 10 minutes to degrade RNA template.

The volume was brought to 50 uL (i.e., add 26 uL H₂O).

5. One Choma-Spin+TE 30 column (Clontech, #K1321) was prepared for every probe made.

Air bubbles were removed from the column.
b. The break-away end of the column was removed and the column placed in an empty 2 mL tube and spun for 5 minutes at 700 g (in Eppendorf 5415C “3.5”).
c. The column was removed and the flow-through discarded. The column was placed in clean tube. The probe was added slowly to the center of the column bed without disturbing the matrix so that the liquid did not touch the side of the column and flow down the edge of the column wall.
   The probe was eluted by spinning the column as above.

### Hybridization

1. The hybridization chamber was preheated to 65° C.

2. 10 mL of 10% Formamide Church Buffer was added. This was placed in the hybridization chamber for around 15 minutes.

3. Sheared salmon sperm DNA was denatured at 95° C. for 5 minutes, placed on ice, and then added to the hybridization mixture at a final concentration of 100 ug/mL. Prehybridization was for 1.5 hours.

4. The amount of probe was calculated necessary to achieve 2×106 cpm/mL for 10 mL.

The Cot Annealing Reactions (per bottle) were as follows:
Rat probe with Rat Filters:
10 ug Poly dA (>200 nt)
10 ug Rat Cot 10 DNA
25 uL 20×SSC
probe+water to 100 uL

Mouse probe with Rat Filters:
10 ug Poly dA (>200 nt)
10 ug Mouse Cot 1 DNA
25 uL 20×SSC
probe+water to 100 uL
Also added 5 ug Rat Cot 10 DNA to the prehybridization.

Human probe with Human Filters:
10 ug Poly dA (>200 nt)
10 ug Human Cot 1 DNA
25 uL 20×SSC
probe+water to 100 uL
   The probe was heated to 95° C., and then probe was allowed to preanneal at 65° C., for 1.5 hours.

6. The probe was added to prehybridizing filters (directly to the solution and not onto the filters) and hybridization was for approximately 20 hours.

### Washing

1. Probe was removed.

2. Three quick washes were performed with preheated 2×SSC/1% SDS, 65° C. (washes could be done in roller bottles).

3. Two washes were performed for 15 minutes each with preheated high stringency wash buffer:
0.5×SSC, 0.1% SDS for cross species washes
0.5×SSC, 0.1% SDS for normal washes
0.1×SSC, 0.1% SDS for very high stringency washes

4. After the high stringency washes, the filters were rinsed in a large square petri dish in 2×SSC, no SDS. For experiments in which many filters are used, the 2×SSC is frequently changed so there is no residual SDS left on the filters.

5. The filters were removed from the 2×SSC and placed on Whatman filter paper. Filters were baked at 85° C. for 1 hour or longer. Screens were protected against any moisture. Filters were placed on a blank phosphorimager screen. No yellowed phosphoimager screens were used since they may not respond to exposure linearly. Screens had been erased on a light box for no less than 20 minutes.

6. Blots were exposed to the screen at least 48 hours or as necessary.

### Scanning Filters on Fuji Phosphorimager

1. Gradation 16 bit, Resolution 50m, Dynamic Range 54000, select Read and Launch Image Gauge. Image was saved on the hard drive.

### Appendix I

### 10% Formamide-Church Buffer:

59.6 mL water

70 mL 20% SDS

50 mL 2M NaPO4 pH 7.2

20 mL Ultrapure Formamide

0.4 mL 0.5M EDTA pH 8.0

The above components were added to water, mixed, and filtered through a 0.2 um filter.

### RT-PCR Protocol

1. For one PCR reaction mix, the following components were used:
28 ul 5X First Strand Buffer
14 ul 0.1M DTT
4 ul dNTPs (20 mM)
7 ul Rnase Inhibitor
7 ul Superscript II
   This buffer can be stored at −80° C. for 3 months.

2. Total RNA was reversed transcribed as follows:
1.4 ug Total RNA (DNAsed)
14 ul Random Primers (50 ng/ul—Gibco)
   Water was added to 60 ul. The mixture was incubated at 70° C. for 10 minutes and then placed on ice for 2 minutes. 60 ul of the RT Reaction Mix was added. Incubation was at room temperature for 10 minutes, then 50° C. for 30 minutes, then 90° C. for 10 minutes. The sample was diluted with 480 ul water to result in 10 ng per 5 ul.

3. The PCR reaction was performed with the following ingredients:
5 ul 4×PCR Buffer
5 ul cDNA (at 10 ng/5 ul)
5 ul 1 uM Primer Pair
5 ul Enzyme Cocktail (0.2 ul Hot Start Tag, 1 ul 2 mM dNTPs, 3.8 ul water

Cycling was as follows:
95° C. 15 minutes
94° C. 30 seconds
52° C. 30 seconds
72° C. 1 minute
Cycle 26-30 times
72° C. 10 minutes
4° C. Hold

Cerebellar granule cell isolation was performed according to the method disclosed in Johnson et al. (1996) J. Neurosci. 16:74877-7495.

The induction of apoptosis in neurites induced by kainate is described in Neurosci. 75:675-683 (1996). The procedure shown in this reference was followed.

The following parameters were checked:

(1) Cerebellum granule neuron viability following potassium and serum withdrawal at time points corresponding to PCR-based methods for differential gene expression (Hoechst stain).

(2) Effects of 2 ug/ml actinomycin D on potassium and serum withdrawal at 24 hours on cerebellar granule neurons; viability by Hoeschst stained cell counts.

(3) Time course of kainate-induced cell death for parallel analysis of PCR-based method for differential gene expression of CGN Poly A mRNA.

(4) Time course of kainate-induced (30 minute exposure) apoptosis in CGNs; analysis by Hoechst cell counts.

(5) Time course of potassium withdrawal apoptosis in CGNs in defined media for PCR-based method for differential gene expression of analysis by Hoechst counts.

While this invention has been particularly shown and described with reference to preferred embodiments thereof, it will be understood by those skilled in the art that various changes in form and details may be made therein without departing from the spirit and scope of the invention as defined by the appended claims.

What is claimed is:

1. An isolated NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 nucleic acid molecule selected from the group consisting of:
a) a nucleic acid molecule comprising a nucleotide sequence which is at least 60% identical to the nucleotide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34;
b) a nucleic acid molecule comprising a fragment of at least 15 nucleotides of the nucleotide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34;
c) a nucleic acid molecule which hybridizes to a nucleic acid molecule comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34, or a complement thereof, under stringent conditions; and
d) a nucleic acid molecule comprising the nucleotide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34.

2. The isolated nucleic acid molecule of claim 1, which is the nucleotide sequence SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34.

3. A host cell which contains the nucleic acid molecule of claim 1.

4. An isolated NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 polypeptide selected from the group consisting of:
a) a polypeptide which is encoded by a nucleic acid molecule comprising a nucleotide sequence which is at least 60% identical to a nucleic acid comprising the nucleotide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34;
b) a polypeptide encoded by a nucleic acid molecule which hybridizes to a nucleic acid molecule comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34, or a complement thereof under stringent conditions; and
c) a polypeptide which is encoded by a nucleic acid comprising the nucleotide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34.

5. An antibody which selectively binds to a polypeptide of claim 4.

6. The polypeptide of claim 4, further comprising heterologous amino acid sequences.

7. A method for producing a polypeptide selected from the group consisting of:
a) a polypeptide encoded by a nucleic acid molecule comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34; and
b) a polypeptide encoded by a nucleic acid molecule which hybridizes to a nucleic acid molecule comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34;
comprising culturing the host cell of claim 3 under conditions in which the nucleic acid molecule is expressed.

8. A method for detecting the presence of a nucleic acid molecule of claim 1 or a polypeptide encoded by the nucleic acid molecule in a sample, comprising:
a) contacting the sample with a compound which selectively hybridizes to the nucleic acid molecule of claim 1 or binds to the polypeptide encoded by the nucleic acid molecule; and
b) determining whether the compound hybridizes to the nucleic acid or binds to the polypeptide in the sample.

9. A kit comprising a compound which selectively hybridizes to a nucleic acid molecule of claim 1 or binds to a polypeptide encoded by the nucleic acid molecule and instructions for use.

10. A method for identifying a compound which binds to a polypeptide or modulates the activity of the polypeptide of claim 4 comprising the steps of:
a) contacting a polypeptide, or a cell expressing a polypeptide of claim 4 with a test compound; and
b) determining whether the polypeptide binds to the test compound or determining the effect of the test compound on the activity of the polypeptide.

11. A method for modulating the activity of a polypeptide of claim 4 comprising contacting the polypeptide or a cell expressing the polypeptide with a compound which binds to the polypeptide in a sufficient concentration to modulate the activity of the polypeptide.

12. A method for identifying a compound capable of treating a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity, comprising assaying the ability of the compound to modulate NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 nucleic acid expression or NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 polypeptide activity, thereby identifying a compound capable of treating a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity.

13. A method of identifying a nucleic acid molecule associated with a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity, comprising:
a) contacting a sample from a subject with a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity, comprising nucleic acid molecules with a hybridization probe comprising at least 25 contiguous nucleotides of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34, defined in claim 2; and
b) detecting the presence of a nucleic acid molecule in the sample that hybridizes to the probe, thereby identifying a nucleic acid molecule associated with a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity.

14. A method of identifying a polypeptide associated with a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity, comprising:
a) contacting a sample comprising polypeptides with a NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25, polypeptide defined in claim 4; and
b) detecting the presence of a polypeptide in the sample that binds to the NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 binding partner, thereby identifying the polypeptide associated with a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity.

15. A method of identifying a subject having a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity, comprising:
a) contacting a sample obtained from the subject comprising nucleic acid molecules with a hybridization probe comprising at least 25 contiguous nucleotides of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 defined in claim 2; and
b) detecting the presence of a nucleic acid molecule in the sample that hybridizes to the probe, thereby identifying a subject having a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity.

16. A method for treating a subject having a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity, or a subject at risk of developing a disorder characterized by aberrant NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 activity, comprising administering to the subject a NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 modulator of the nucleic acid molecule defined in claim 1 or the polypeptide encoded by the nucleic acid molecule or contacting a cell with a NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 modulator.

17. The method of claim 16, wherein the NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 modulator is a small molecule; peptide; phosphopeptide; anti-NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 antibody; a NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 polypeptide comprising the amino acid sequence of SEQ ID NO:2, 4, 10, 11, 12, 13, 52 or 54, or a fragment thereof; a NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 polypeptide encoded by a nucleic acid molecule which hybridizes to a complement of a nucleic acid molecule consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 at 6×SSC at 45° C., followed by one or more washes in 0.2×SSC, 0.1% SDS at 65° C.

18. The method of claim 16, wherein the NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 modulator is
a) an antisense NARC SC1, NARC 10A, NARC 1, NARC 12, NARC 13, NARC17, NARC 25, NARC 3, NARC 4, NARC 7, NARC 8, NARC 11, NARC 14A, NARC 15, NARC 16, NARC 19, NARC 20, NARC 26, NARC 27, NARC 28, NARC 30, NARC 5, NARC 6, NARC 9, NARC 10C, NARC 8B, NARC 9, NARC2A, NARC 16B, NARC 1C, NARC 1A, or NARC 25 nucleic acid molecule;
b) a ribozyme;
c) the nucleotide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 or a fragment thereof;
d) a nucleic acid molecule which hybridizes to a complement of a nucleic acid molecule consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34, at 6×SSC at 45° C., followed by one or more washes in 0.2×SSC, 0.1% SDS at 65° C.; or
f) a gene therapy vector.
